(19) United States
(12) Patent Application Publication
(10) Pub. No.: US 2022/0395500 A1
(43) Pub. Date: Dec. 15, 2022

(54) SMALL MOLECULE ACTIVATORS OF INTERFERON REGULATORY FACTOR 3 AND METHODS OF USE THEREOF

(71) Applicant: Neuralexo, Inc., Portland, OR (US)

(72) Inventors: Susan Stevens, Portland, OR (US); Mary Stenzel-Poore, Portland, OR (US); Marie Foss, Portland, OR (US); Aaron Nilsen, Portland, OR (US); Haihong Jin, Portland, OR (US)

(21) Appl. No.: 17/307,851

(22) Filed: May 4, 2021

Related U.S. Application Data
(60) Provisional application No. 63/019,925, filed on May 4, 2020.

(51) Int. Cl. A61K 31/496; A61P 9/10; A61K 31/167; A61K 31/519; A61K 31/5377; A61K 31/341; A61K 31/381; A61K 31/423; A61K 31/428; A61K 31/427; A61K 31/4365; A61K 31/437; A61K 31/4439; A61K 31/4245; A61K 31/4178; A61K 31/55

(52) U.S. Cl. CPC A61K 31/496; A61B 2017/22082; A61K 31/167; A61K 31/519; A61K 31/5377; A61K 31/341; A61K 31/381; A61K 31/423; A61K 31/428; A61K 31/427; A61K 31/4365; A61K 31/437; A61K 31/4439; A61K 31/4245; A61K 31/4178; A61K 31/55; A61P 9/10

(57) ABSTRACT

Small molecule activators of interferon regulatory factor (IRF), such as IRF3, and methods of use are provided. In particular, compositions and methods for upregulating interferon regulatory factor 3 (IRF3) activity, such as in the brain following stroke to provide potent protection against ischemic brain injury, to improve a therapeutic time window for providing treatments to stroke patients and/or for enhancement of vaccine platforms are disclosed.

### CROSS REFERENCE TO RELATED APPLICATION

This application claims the benefit of U.S. provisional patent application No. 63/019,925 filed May 4, 2020, the entire disclosure of which is incorporated herein by reference.

### ACKNOWLEDGEMENT OF GOVERNMENT SUPPORT

This invention was made with government support under NS095538 and NS062381 awarded by National Institutes of Health. The government has certain rights in the invention.

### FIELD

This disclosure relates to interferon regulatory factors (IRFs), and in particular, to compositions and methods for upregulating interferon regulatory factor 3 (IRF3) activity, such as in the brain following stroke to provide potent protection against ischemic brain injury, to improve a therapeutic time window for providing treatments to stroke patients and/or for enhancement of vaccine platforms.

### BACKGROUND

Stroke causes neuronal injury and death due to deprivation of oxygen and nutrients that are essential for cell survival. In addition, inflammatory mediators are released that trigger a cascade of responses that exacerbate injury. Although these injurious pathways are well defined, it has been a major challenge to identify ways to mitigate these pathways to reduce ischemic damage.

Stroke imposes a significant financial burden on the United States and the world population as a whole. According to some estimates, eighty-five percent of all strokes are classified as ischemic strokes, associated with vascular obstruction of cerebral blood flow. Although hundreds of therapies have been tested, few ischemic stroke treatments have shown efficacy. Examples include thrombolytics (e.g., tissue plasminogen activator (tPA)) and clot removal treatments (e.g., mechanical thrombectomy). Both treatments focus on minimizing neurological damage through restoration of blood flow to the affected tissue by prompt removal of the vascular obstruction.

Unfortunately, there is a narrow therapeutic window (less than 6 hours) in which thrombolytic and/or thrombectomy treatments can be utilized, as the affected neural tissue cannot survive beyond this time without needed oxygen and nutrients. Although it is estimated that 1.9 million neurons are lost each minute a stroke is untreated, these treatments can only be applied once a patient is evaluated in the hospital for contraindications (e.g., type of stroke) in the case of thrombolytics, or prepared for surgery in the case of mechanical thrombectomy, both of which take valuable time. Thus overall, less than 10% of patients are eligible for either of thrombolytics or mechanical thrombectomy treatments. As such, effective means of treating stroke are limited, due at least in part to a small time window in which effective treatments such as thrombolytics and mechanical thrombectomy can be used to reduce adverse consequences of stroke conditions.

### SUMMARY

Disclosed herein are IRF modulatory agents and methods of using such agents to treat patients suffering from a stroke condition. In one embodiment, a method of altering activity and/or expression of an IRF in a subject suffering from a stroke condition is disclosed. The method includes administering to the subject an effective amount of an IRF modulatory agent comprising one or more compounds listed in Tables 7-8. In another embodiment, the IRF modulatory agent comprises one or more compounds of the general formulas shown at any one of Tables 1-6. Altering activity and/or expression of the IRF includes increasing the IRF activity and/or expression as compared to IRF activity and/or expression prior to or in lieu of administration of the IRF modulatory agent. Increasing expression or activity of the IRF reduces and/or inhibits one or more signs or symptoms associated with the stroke, thereby treating the stroke condition.

Also disclosed is a method of treating a subject suffering from a stroke which is at least in part regulated or regulatable by activity and/or expression of an IRF, such as IRF3. The method includes contacting a cell or cells of the subject with an effective amount of an IRF modulatory agent comprising one or more compounds listed in Tables 7-8, to increase the activity and/or expression of the IRF, thereby treating the stroke. In another embodiment, the IRF modulatory agent comprises one or more compounds of the general formulas shown at any one of Tables 1-6. In one embodiment, contacting the cell or cells of the subject with the IRF modulatory agent delays a depletion of cellular energy stores and delays membrane depolarization of the cell or cells affected by the stroke as compared to a rate at which depletion of cellular energy stores and membrane potential depolarization occurs in the absence of the cell or cells being contacted with the IRF modulatory agent.

Also disclosed is a method of increasing a time frame of a therapeutic window in which one or more treatments can be effectively provided to a subject suffering from an acute ischemic event. The method includes altering activity and/or expression of an IRF, such as IRF3, by administering to the subject an IRF modulatory agent, such as an IFR3 modulatory agent, comprising one or more compounds encompassed by the Tables 7-8 within a predetermined period of time of an initiation of the acute ischemic event. In another embodiment, the IRF modulatory agent comprises one or more compounds of the general formulas shown at any one of Tables 1-6. In one embodiment, increasing the time frame of the therapeutic window comprises improving a tolerance of neural tissue to the acute ischemic event, as compared to the tolerance to the acute ischemic event which would otherwise occur in the absence of administering to the subject the IRF modulatory agent. In some examples, the one or more treatments include administration of a thrombolytic agent and mechanical thrombectomy.

Although the above disclosed methods have been described with respect to stroke, other conditions/disorders and/or diseases can be treated via similar methodology that includes altering expression and/or activity of IRFs via administration of one or more of the IRF modulatory agents, such as IRF3 modulatory agents, selected from the compounds listed in Tables 7-8. In some examples, the one or more IRF modulatory agents comprise one or more compounds of the general formulas shown at any one of Tables 1-6.

For example, disclosed is a method of altering activity and/or expression of an IRF in a subject suffering from a condition or disease associated with IRF comprising administering to the subject an effective amount of an IRF modulatory agent comprising one or more compounds listed in Tables 7-8. In some examples, the IRF modulatory agent comprises one or more compounds of the general formulas shown at any one of Tables 1-6.

In one example, the condition or disease is cancer. In another example, the condition or disease is multiple sclerosis. In another example, the condition or disease is chronic fatigue syndrome. In another example, the condition or disease is an immune response to an antigen.

Also provided are methods of treating a subject having a condition/disorder or disease that is at least in part regulated by activity and/or expression of an IRF, such as IRF3, comprising contacting a cell or cells of the subject with an effective amount of an IRF modulatory agent, such as an IRF3 modulatory agent, comprising one or more compounds one or more compounds listed in Tables 7-8, to increase the activity and/or expression of the IRF, such as increase IRF3 activity and/or expression, thereby treating the condition/disorder or disease. In some examples, the IRF modulatory agent comprises one or more compounds of the general formulas shown at any one of Tables 1-6. As one example, the condition or disease is cancer. In another example the condition or disease is multiple sclerosis. In another example the condition or disease is chronic fatigue syndrome. In another example the condition or disease is an immune response to an antigen.

The foregoing and other features of the disclosure will become more apparent from the following detailed description, which proceeds with reference to the accompanying figures.

### BRIEF DESCRIPTION OF THE DRAWINGS

FIG. 1A shows a Venn diagram comparing reprogrammed genes in response to three preconditioning paradigms, lipopolysaccharide (LPS), cytosine-phosphate-guanine (CpG) and brief ischemia (IP), illustrating that following stroke, subjects treated with any one of the three preconditioning paradigms share a genomic fingerprint comprised of 12 genes.

FIG. 1B shows a hypothetical gene-transcriptional regulatory element (TRE) network of genes common to all three preconditioning paradigms of FIG. 1A, showing the relationship of identified TREs to the regulated genes.

FIG. 2A is a bar graph illustrating that the preconditioning effect of LPS is completely abbrogated in IRF3 or IRF7 knock-out (KO) mice in which ischemia was induced by middle cerebral artery occlusion (MCAO). IRF3 deficient and IRF7 deficient mice were preconditioned with LPS or saline 72 hours prior to MCAO. Infarct volume was measured 24 hours after surgery. Group mean+/−SEM is shown; ***p<0.001.

FIG. 2B is a bar graph illustrating brain gene transcription profiles for a subset of the 12-induced genes (refer to FIG. 1A) in IRF3 KO and IRF7 KO mice in the context of stroke. Gene transcription was measured in the brain 24 hours post MCAO in LPS preconditioned wildtype, IRF3 deficient, and IRF7 deficient mice using quantitative PCR. Data is represented as fold change vs genotype matched saline+MCAO (+/−SD; n=3-4 mice/genotype).

FIG. 2C is a bar graph illustrating that the preconditioning effect of CpG is completely abbrogated in IRF7 KO mice in which ischemia was induced by MCAO. WT and IRF7 deficient mice were preconditioned with CpG, LPS, or saline 72 hours before MCAO. Infarct volume was measured 24 hours following surgery. Group mean+SEM is shown; ***p<0.001.

FIG. 2D is a bar graph illustrating that the preconditioning effect of CpG is completely abbrogated in IRF3 KO mice in which ischemia was induced by MCAO. WT and IRF3 deficient mice were preconditioned with CpG, or saline 72 hours before MCAO. Infarct volume was measured 24 hours following surgery. Group mean+SEM is shown; ***p<0.001.

FIG. 3 is a bar graph illustrating the role of interferon-induced protein with tetratricopeptide repeats 1 (IFIT1) in LPS preconditioning against ischemic injury in mice. IFIT1-deficient mice were preconditioned with LPS three days prior to MCAO and neuroprotection was completely abrogated in the IFIT1-deficient mice, indicating that IFIT1 is required for LPS preconditioning-induced protection. Infarct volume was measured 24 hours after surgery. Group mean+/−SEM is shown; ** p<0.01.

FIG. 4 is a bar graph illustrating that treatment of poly ICLC (PIC) after exposure to oxygen glucose deprivation (OGD) results in significant protection when cell death was examined 24 hours later. Primary cortical cultures were exposed to OGD after which poly ICLC was added. Cell death was assessed 24 hours later. **p<0.01 ***p<0.001. Data reflect mean+/−SEM.

FIG. 5A is a graph showing that dsRNA and DMXAA are potent activators of interferon-stimulated response element (ISRE). Using a 96-well plate format, mouse macrophage cells (RAW264.7) containing an ISRE-firefly luciferase reporter with a constitutively active renilla luciferase internal control for normalization (Promega) were treated with various compounds including dsRNA, DMXAA and PIC for 3 hours and luciferase activity was determined.

FIG. 5B is a dose-response curve of an expanded dose response study in RAW264.7 cells containing the ISRE-firefly luciferase reporter with the constitutively active renilla luciferase internal control for normalization, for PIC, dsRNA, and dimethylxanthenone-4-acetic acid (DMXAA). Data is depicted as fold increase ISRE activity vs matched vehicle, plotted against log[agonist] (M). DMXAA was shown to have superior drug-like properties for activating mouse ISRE (EC50˜16 μM).

FIG. 6A is a bar graph depicting the response of a mouse neuronal cell line (Neuro2a) to DMXAA and 5′ppp dsRNA with regard to IRF-related genes (RSAD2 and IFIT1) and inflammatory pathway genes (TNF and IL-6). Neuro2a cells were treated with 5′ppp dsRNA or DMXAA (10 ug/ml) for 3 hours. RNA was collected from cell pellets and qtPCR was performed with indicated primers. Data is depicted as mRNA fold-change versus vehicle. Values are normalized to actin and are presented as fold change over vehicle control.

FIG. 6B is a bar graph depicting the response to a microglial cell line (BV2) to DMXAA and 5′ppp dsRNA with regard to IRF-related genes (RSAD2 and IFIT1) and inflammatory pathway genes (TNF and IL-6). BV2 cells were treated with 5′ppp dsRNA or DMXAA (10 ug/ml) for 3 hours. RNA was collected from cell pellets and qtPCR was performed with indicated primers. Data is depicted as mRNA fold-change versus vehicle. Values are normalized to actin and are presented as fold change over vehicle control.

FIG. 7A is a bar graph depicting DMXAA-induced induction of IRF-related gene expression in mice. Male C57/BL6 mice were given DMXAA (2 mg/kg or 10 mg/kg, IP) and brain cortical tissue was collected at 3 hours. RNA was extracted and qtPCR was performed with indicated primers. Data is depicted as mRNA fold-change versus vehicle. Values depicted are group means+/−SEM; n=4 per group.

FIG. 7B is a plot showing that wild-type mice treated with DMXAA show a significant reduction in ischemic injury compared to controls, and that mice lacking IRF3 are not protected by treatment with DMXAA. Acute stroke efficacy of DMXAA was observed in mice given 250 μg DMXAA (IP) immediately following 60 minutes MCAO. Infarct volumes were measured at 24 hours. Shown for each group are individual data points+/−group SEM, n=7-8 per group, ** p<0.01.

FIG. 8 provides a small library of compounds similar to DMXAA, FAA and L56. The small library also included acridines. For the depicted compounds, R is one or more aromatic ring substituents and Ar is an aromatic or heteroaromatic ring. The small library was used to conduct a screening of a human THP1 dual ISRE/NFκB reporter cell line subsequent to appropriate assay conditions being determined for the reporter cell line.

FIG. 9A depicts an image of a structure of AV-C, a known human-specific IRF activator.

FIG. 9B is an EC50 (50% effective concentration) graph of AV-C dependent IRF activation in THP1-Dual cells, determined with an 8-point dose response experiment. IRF activation was monitored by using an IRF-Lucia luciferase reporter construct. The detection of AV-C dependent IRF-Lucia luciferase induction was via use of detection reagent QUANTI-Luc (Invivogen), and luminescence was measured within 5 minutes of addition of the detection reagent. The EC50 for AV-C dependent IRF activation is 5.3 μM.

FIG. 9C is a CC50 (50% cytotoxic concentration) graph of AV-C dependent IRF activation in THP1-Dual cells, determined with an 8-point dose response experiment. Cell viability was monitored based on quantitation of ATP present, as an indicator of metabolically active cells, where luminescence is proportional to the amount of ATP present, using Cell-Titer Glo (Promega). The CC50 for AV-C is 7.9 μM.

FIG. 10A is a representative plot for hit assessment of one THP IRF-Lucia plate from a THP1-Dual pilot library screen using the Spectrum Collection Library (MicroSource Discovery Systems, Inc). FIG. 10A shows percent activation of the IRF-Lucia reporter for compounds from the Spectrum Collection Library normalized to LPS activation of the IRF-Lucia reporter, where LPS activation was set as 100%, and test compound percent activation is reported as percent of LPS activation. Samples with percent activation above 3a were labeled as hits.

FIG. 10B is a graph illustrating the distribution of the test samples shown at FIG. 10A about the mean (a), illustrating that the output of the assay discussed at FIG. 10A follows a normal distribution.

FIG. 11 is a bar graph showing that the human-specific IRF activator AV-C induces robust IFIT1 and RSAD2 expression, with no increase in TNF expression. SH-SY5Y cells were treated with 20 μM AV-C for 3 hours. RNA was collected from cell pellets and qtPCR was performed with indicated primers. Values at FIG. 11 are normalized to actin and presented as fold change over vehicle control.

FIG. 12 is a flow chart depicting a general process flow for identification of lead compounds using the high throughput screening platform of the present disclosure.

FIG. 13 is a schematic of the general process flow of FIG. 12 in greater detail.

FIG. 14A is an exemplary data plot from 40, 384-well plates of a high throughput test compound screen of human THP1 monocyte cells containing the IRF-Lucia luciferase and NFκB-SEAP (secreted embryonic alkaline phosphatase) dual reporter (THP1-Dual, Invitrogen), to enable the simultaneous study of the IRF and NFκB signaling pathways, respectively. The Lucia luciferase reporter gene is under the control of an ISG54 (interferon-stimulated gene) minimal promoter in conjunction with five interferon-stimulated response elements (ISRE). ISRE % activation for each test compound (individual circles) is shown. Depicted is data for the IRF-Lucia luciferase reporter. Compounds were assigned as hits if percent ISRE activation exceeded 66 above the mean for the batch.

FIG. 14B is a counterscreen of the high throughput test compound screen discussed at FIG. 14A, to further select compounds with reduced NF-κB-coupled SEAP activation. The SEAP gene is driven by an IFNβ minimal promoter fused to five copies of the NFκB consensus transcriptional response element, and three copies of the c-Rel binding site. The graph at FIG. 14B represents the percent NF-κB activation of all combined hits from the 40 primary HTS plates discussed with regard to FIG. 14A. Cutoffs for inclusion of hits with NF-κB activation above 2σ were set based on their ISRE/NF-κB or ISRE-NF-κB signal scores.

FIG. 14C is a schematic illustrating a combined list of counterscreened compounds as discussed with regard to FIG. 14B called by either Z-score or percent activation (PA) methods. 122 compounds were found to be shared between the two methods (Z-score and PA).

FIG. 15 is a plot showing correlation between the IRF activation observed in a cherry-picking confirmation screen and a singlicate primary screen measurement.

FIG. 16A is a chromatagraph of NA-42 (4-((3-ethoxybenzyl)oxy)-N-(4-(4-(thiophene-2-carbonyl)piperazin-1-yl)phenyl)benzamide), along with its chemical structure and mass determination.

FIG. 16B is a reaction scheme for synthesis of NA-42.

FIG. 17A is a chromatagraph of ND-13 (N-(5-(5,6-dimethylbenzo[d]oxazol-2-yl)-2-methylphenyl)-4-methoxy-3-nitrobenzamide), along with its chemical structure and mass determination.

FIG. 17B is a reaction scheme for synthesis of ND-13.

FIG. 18A is an 8-point dose response curve showing NF-κB-SEAP and IRF-Lucia luciferase reporter construct activity in human THP1 cells and mouse J774 cells as a function of NA-42 concentration. Depicted are group mean+/−standard deviation.

FIG. 18B shows values corresponding to EC50, selectivity index (IRF3 activity/NF-κB activity), and toxicity index (CC50/EC50) for NA-42. EC50 (50% effective concentration of NA-42 dependent IRF activation) was determined in THP1-Dual cells, with IRF activation based on IRF-Lucia luciferase induction. The CC50 (50% cytotoxic concentration of NA-42) was determined in THP-1 Dual cells, with cell viability monitored based on amount of ATP present as a function of NA-42 concentration. The selectivity index is based on maximum response of IRF3 activity over maximum response of NF-κB activity.

FIG. 19A is an 8-point dose response curve showing NF-κB-SEAP and IRF-Lucia luciferase reporter construct activity in human THP1 cells and mouse J774 cells as a function of ND-13 concentration. Depicted are group mean+/−standard deviation.

FIG. 19B shows values corresponding to EC50, selectivity index (IRF3 activity/NF-κB activity), and toxicity index (CC50/EC50) for ND-13. EC50 (50% effective concentration of ND-13 dependent IRF activation) was determined in THP1-Dual cells, with IRF activation based on IRF-Lucia luciferase induction. The CC50 (50% cytotoxic concentration of ND-13) was determined in THP-1 Dual cells, with cell viability monitored based on amount of ATP present as a function of ND-13 concentration. The selectivity index is based on maximum response of IRF3 activity over maximum response of NF-κB activity.

FIG. 20 is a graph of IRF3 dependent induction of IP10 in mouse bone marrow derived macrophages (mBMDMs), as a function of increasing concentrations of NA-42. IP-10 levels were determined by mouse CXCL10/IP10 ELISA (Invitrogen, Carlsbad, Calif.) and are expressed as pg/ml. Data represents group mean+/−standard deviation.

FIG. 21 is a graph of IRF3 dependent induction of IP10 in mouse bone marrow derived macrophages (mBMDMs), as a function of increasing concentrations of ND-13. IP-10 expression was determined by mouse CXCL10/IP10 ELISA (Invitrogen, Carlsbad, Calif.), and is expressed as pg/ml. Data represents group mean+/−standard deviation.

FIG. 22A shows a series of graphs of IRF3 dependent cytokine induction by NA-42, IFNb and DMXAA for cytokines IL6, MCP3, Rantes, and TNFa vs vehicle. Cytokine induction is shown for both wild-type mouse mBMDMs and IRF3 KO mouse mBMDMs. Cytokine expression was measured using a cytokine 20-plex mouse ProcartaPlex kit (Invitrogen, Carlsbad, Calif.) for labeling of multiple cytokines within an individual sample and run on a Luminex (Austin, Tex.) 200 instrument. Data is expressed as pg/ml. Shown are group means+/−SEM; *p<0.5, **p<0.01, ***p<0.001, ****p<0.0001.

FIG. 22B shows a series of graphs of IRF3 dependent cytokine induction by NA-42, IFNb and DMXAA for cytokines MCP1, Mip1b, Mip1a, and Gro-alpha vs vehicle. Cytokine induction is shown for both wild-type mouse mBMDMs and IRF3 KO mouse mBMDMs. Cytokine expression was measured using a cytokine 20-plex mouse ProcartaPlex kit (Invitrogen) for labeling of multiple cytokines within an individual sample and run on a Luminex 200 instrument. Data is expressed as pg/ml. Shown are group means+/−SEM; *p<0.5, **p<0.01, ***p<0.001, ****p<0.0001.

FIG. 23A shows a series of graphs of IRF3 dependent cytokine induction by ND-13, IFNb and DMXAA for cytokines IL6, MCP3, Rantes, and TNFa vs vehicle. Cytokine induction is shown for both wild-type mouse mBMDMs and IRF3 KO mouse mBMDMs. Cytokine expression was measured using a cytokine 20-plex mouse ProcartaPlex kit (Invitrogen) for labeling of multiple cytokines within an individual sample and run on a Luminex 200 instrument. Data is expressed as pg/ml. Shown are group means+/−SEM; *p<0.5, **p<0.01, ***p<0.001, ****p<0.0001.

FIG. 23B shows a series of graphs of IRF3 dependent cytokine induction by ND-13, IFNb and DMXAA for cytokines MCP1, Mip1b, Mip1a, and Gro-alpha vs vehicle. Cytokine induction is shown for both wild-type mouse mBMDMs and IRF3 KO mouse mBMDMs. Cytokine expression measured using a cytokine 20-plex mouse ProcartaPlex kit (Invitrogen) for labeling of multiple cytokines within an individual sample and run on a Luminex 200 instrument. Data is expressed as pg/ml. Shown are group means+/−SEM; *p<0.5, **p<0.01, ***p<0.001, ****p<0.0001.

FIG. 24 is a graph of NA-42 dose dependent IP-10 induction in human monocyte-derived macrophages. IP-10 expression was monitored by Human CXCL10/IP10 ELISA (R&D Systems, Minneapolis, Minn.) and is expressed as pg/ml. Shown are group means+/−standard deviation; ****p<0.0001.

FIG. 25 is a graph of ND-13 dose dependent IP-10 induction in in human monocyte-derived macrophages. IP-10 expression was monitored by Human CXCL10/IP10 ELISA (R&D Systems, Minneapolis, Minn.) and is expressed as pg/ml. Shown are group means+/−standard deviation; ****p<0.0001.

FIG. 26A is a schematic of an exemplary cell-signalling pathway that includes TRIF, along with TLR4, TLR3, IPS1, STING and IRF3.

FIG. 26B is a bar graph illustrating that NA-42 dependent induction of IRF3 is TRIF dependent. Shown is fold-change of an interferon dependent luciferase reporter expressed in various telomerized human fibroblasts (THF) cell lines lacking key adapter molecules in the IRF3 activating pathway (WT THF, IPS1 KO, STING KO, and TRIF KO) in response to different treatments (untreated, Sendai 1/100, 313 ng/ml LPS, 23 μM NA-42). Shown are group means+/−SEM.

FIG. 26C is a bar graph illustrating that ND-13 dependent induction of IRF3 is TRIF dependent. Shown is fold-change of an interferon dependent luciferase reporter expressed in various telomerized human fibroblasts (THF) cell lines lacking key adapter molecules in the IRF3 activating pathway (WT THF, IPS1 KO, STING KO, and TRIF KO) in response to different treatments (untreated, Sendai 1/100, 313 ng/ml LPS, 23 μM NA-42). Shown are group means+/−SEM.

FIG. 27A is a bar graph illustrating the ability of NA-42 to induce plasma cytokine expression in vivo in mice. WT C57/BL6 mice were given either vehicle, DMXAA or NA-42 (10 mg/kg, intravenous), and cytokine expression was determined using a cytokine 20-plex mouse ProcartaPlex kit (Invitrogen) for labeling of multiple cytokines within an individual sample and run on a Luminex 200 instrument. The top panel at FIG. 27A shows induced expression of IL-6, and the bottom panel shows induced expression of MCP1. Expression is represented as pg/ml.

FIG. 27B is a bar graph illustrating the ability of ND-13 to induce plasma cytokine expression in vivo in mice. WT C57/BL6 mice were given either vehicle, DMXAA or ND-13 (10 mg/kg, intravenous), and cytokine expression was determined using a cytokine 20-plex mouse ProcartaPlex kit (Invitrogen) for labeling of multiple cytokines within an individual sample and run on a Luminex 200 instrument. The top panel at FIG. 27B shows induced expression of IL-6, and the bottom panel shows induced expression of MCP1. Expression is represented as pg/ml.

FIG. 28 shows a bar graph illustrating that NA-42 and ND-13 enhance antibody titers responsive to a Chikungunya Virus (CHIKV) challenge. Conditions shown include virus-like particle (VLP) alone, VLP+5 mg/kg ND13, VLP+10 mg/kg ND13, VLP+5 mg/kg NA-42, and VLP+10 mg/kg NA-42. Depicted is Log 10 antibody titer representing anti-CHIKV Total IgG. Individual samples are depicted as filled circles for each condition. Bar graphs represent average geometric mean titers for each treatment cohort+standard deviation.

FIGS. 29A-29B illustrate characterization of NA-42 (FIG. 29A), ND-13 (FIG. 29B) and ND-95 (FIG. 29B).

### DETAILED DESCRIPTION OF SEVERAL EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Unless otherwise noted, technical terms are used according to conventional usage. Definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710); and other similar references. The singular terms (“a”, “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. All publications, patent applications, patents, and other references mentioned herein are incorporated by reference in their entirety. All sequences provided in the disclosed Genbank Accession numbers are incorporated herein by reference as available on May 4, 2020. In case of conflict, the present specification, including explanations of terms, will control. In addition, the materials, methods, and examples are illustrative only and not intended to be limiting.

### I. Overview of Several Embodiments

Disclosed herein are IRF modulatory agents and methods of using such to treat a condition/disorder or disease for which altering expression and/or activity of an IRF improves at least a sign, symptom or other clinically relevant parameter in a subject suffering from the condition/disorder or disease.

In some embodiments, a method of altering activity and/or expression of an interferon regulatory factor (IRF) in a subject suffering from a condition or disease comprises administering to the subject an effective amount of an IRF modulatory agent, the IRF modulatory agent comprising one or more compounds encompassed by Tables 7-8. In another embodiment, the one or more compounds may comprise a compound or compound of the form of a formula or formulas shown at Tables 1-6.

In one such an example of the method, the IRF is IRF3. In another example of the method, the IRF is IRF7.

In some examples of the method, altering activity and/or expression of the IRF further comprises increasing the IRF activity and/or expression as compared to IRF activity and/or expression prior to administration of the IRF modulatory agent.

In an embodiment of the method, the condition or disease is a stroke. In such an example, administration of the IRF modulatory agent improves a tolerance of neural tissue in the subject to an ischemic event associated with the stroke as compared to the tolerance in the absence of administration of the IRF modulatory agent. In another example where the condition or disease is stroke, the method further comprises providing the subject with a thrombolytic therapy within a first predetermined time period of administration of the effective amount of the IRF modulatory agent. In yet another example of the method where the condition or disease is stroke, the method further comprises performing a surgical thrombectomy on the subject to remove a blood clot from inside an artery or a vein of the subject within a second predetermined time period of administration of the effective amount of the IRF modulatory agent.

In another embodiment of the method, the condition or disease is cancer. In such an example, the cancer comprises one or more of Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kidney Cancer, Klatskin tumor, Krukenberg tumor, Laryngeal Cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple myeloma, Mycosis Fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neurofibroma, Neuroma, Nodular melanoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral cancer, Oropharyngeal Cancer, Osteosarcoma, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, and Wilms' tumor.

In another embodiment of the method, the condition or disease is multiple sclerosis.

In another embodiment of the method, the condition or disease is chronic fatigue syndrome.

In another embodiment of the method the condition or disease is an immune response to an antigen. In such an example, the IRF modulatory agent acts as an adjuvant to potentiate and/or modulate the immune response to the antigen. In another example where the condition or disease is an immune response to an antigen, the the antigen is a chikungunya virus antigen.

In yet another embodiment of the method, the method further comprises selecting the subject suffering from the condition or disease. As an example, selecting the subject with the condition or disease comprises diagnosing the subject with the condition or disease prior to administering the effective amount of the IRF modulatory agent to the subject.

Also disclosed herein is a method of treating a subject having a condition/disorder or disease that is at least in part regulated by activity and/or expression of an interferon regulatory factor (IRF), comprising contacting a cell or cells of the subject with an effective amount of an IRF modulatory agent, the IRF modulatory agent comprising one or more compounds of Table 7 and Table 8, to increase the activity and/or expression of the IRF, thereby treating the condition/disorder of disease. In such an example, the IRF is one or both of IRF3 and IRF7. In another embodiment, the one or more compounds may comprise a compound or compound of the form of a formula or formulas shown at Tables 1-6.

In one embodiment of such a method, increasing the activity and/or expression of the IRF is in relation to IRF activity and/or expression prior to or in the absence of the cell or cells being contacted with the IRF modulatory agent.

As one specific embodiment, the condition/disorder or disease is a stroke. In such an example, contacting the cell or cells of the subject with the IRF modulatory agent delays a depletion of cellular energy stores and delays membrane potential depolarization of the cell or cells affected by the stroke as compared to a rate at which depletion of cellular energy stores and membrane potential depolarization occurs in the absence of the cell or cells being contacted with the IRF modulatory agent. As another example embodiment where the conditions/disorder or disease is a stroke, the method further includes administering to the subject a thrombolytic therapy within a first predetermined time period of the cell or cells being contacted with the IRF modulatory agent. As another example embodiment where the conditions/disorder or disease is a stroke, the method further includes performing a surgical thrombectomy on the subject to remove a blood clot from inside an artery or a vein of the subject within a second predetermined time period of the cell or cells being contacted with the IRF modulatory agent.

In another embodiment of the method, the condition/disorder or disease is a cancer.

In yet another embodiment of the method, the condition/disorder or disease is multiple sclerosis.

In yet another embodiment of the method, the condition/disorder or disease is chronic fatigue syndrome.

In yet another embodiment of the method, the condition/disorder or disease involves an immune response to an antigen. In such an example, the IRF modulatory agent acts as an adjuvant to potentiate and/or modulate the immune response to the antigen. As a specific embodiment, the antigen is a chikungunya virus antigen.

Also disclosed is a method of increasing a time frame of a therapeutic window in which one or more treatments can be effectively provided to a subject suffering from an acute ischemic event, comprising altering activity and/or expression of an interferon regulatory factor (IRF) by administering to the subject an IRF modulatory agent within a predetermined period of time of an initiation of the acute ischemic event. As an example, the IRF is one or more of IRF3 and IRF7.

In one embodiment of such a method, the IRF modulatory agent increases activity and/or expression of the IRF as compared to activity and/or expression of the IRF prior to or in an absence of administration of the IRF modulatory agent.

In another embodiment of such a method, increasing the time frame of the therapeutic window further comprises improving a tolerance of neural tissue to the acute ischemic event in a manner that delays a depletion of cellular energy stores and delays membrane potential depolarization of the neural tissue affected by the acute ischemic event as compared to a rate at which depletion of cellular energy stores and membrane potential depolarization occurs in the absence of administering to the subject the IRF modulatory agent.

In one particular embodiment of the method, the one or more treatments include administration of a thrombolytic agent and mechanical thrombectomy.

As another example embodiment, the method further comprises providing the one or more treatments after administering the IRF modulatory agent, and before the time frame of the therapeutic window elapses.

For such a method, the IRF modulatory agent is one or more of the compounds listed in Tables 7-8. In some embodiments, the one or more compounds comprise a compound or compounds of the general formulas depicted at Tables 1-6.

In an embodiment of such a method, the method includes selecting the subject suffering from the acute ischemic event. Selecting the subject suffering from the acute ischemic event may comprise diagnosing the subject as experiencing the acute ischemic event prior to administering the IRF modulatory agent to the subject.

In any one of the above-described methods and embodiments thereof, the IRF modulatory agent is one or more disclosed herein, including one or more compounds disclosed in Tables 7-8. In some embodiments, the one or more compounds comprise a compound or compounds of the general formulas depicted at Tables 1-6. In some embodiments, the IRF modulatory agent comprises one or more of NA-42 [4-((3-ethoxybenzyl)oxy)-N-(4-(4-(thiophene-2-carbonyl)piperazin-1-yl)phenyl)benzamide]; NA-24 [4-((3-methoxybenzyl)oxy)-N-(4-(4-(thiophene-2-carbonyl)piperazin-1-yl)phenyl)benzamide]; NA-21 [5-bromo-N-{4-[4-(2-thienylcarbonyl)-1-piperazinyl]phenyl}-1-naphthamide]; ND-13 [N-(5-(5,6-dimethylbenzo[d]oxazol-2-yl)-2-methylphenyl)-4-methoxy-3-nitrobenzamide]; ND-95 [4-methoxy-N-(5-(5-(methoxymethoxy)benzo[d]oxazol-2-yl)-2-methylphenyl)-3-nitrobenzamide]; and ND-52 [N-[5-(1,3-benzothiazol-2-yl)-2-methylphenyl]-4-methoxy-3-nitrobenzamide].

In any one of the above-described methods and embodiments thereof, the IRF modulatory agent may be an analog/derivative of any of the disclosed IRF modulatory agents listed in Tables 7-8 (or in other embodiments, an analog/derivative of a compound or compounds of the general formulas depicted at Tables 1-6), which may be designed and synthesized according to the chemical principles known to one of ordinary skill in the art and identified as an IRF modulatory agent by methods known to those of ordinary skill in the art, and in particular with regard to the Examples as disclosed herein.

### II. Terms

In order to facilitate review of the various embodiments of this disclosure, the following explanations of specific terms are provided:

Acyl: A group of the formula RC(O)— wherein R is an organic group.

Acyloxy: A group having the structure —OC(O)R, where R may be an optionally substituted alkyl or optionally substituted aryl. “Lower acyloxy” groups are those where R contains from 1 to 10 (such as from 1 to 6) carbon atoms.

Adjunctive therapy: A treatment used in combination with a primary treatment to improve the effects of the primary treatment. As a representative example, adjunctive therapy includes treatment of a stroke patient with a therapeutic agent that can increase the expression and/or activity of the IRF3 gene or gene product (or other IRF gene product including but not limited to IRF7), where the therapeutic agent is administered prior to, during/concurrently or after a primary treatment (e.g., administration of tissue plasminogen activator (tPA), mechanical thrombectomy, etc.).

Adjuvant: A substance that enhances antigenicity, such as immunostimulatory molecules, including cytokines, costimulatory molecules, and for example, immunostimulatory DNA or RNA molecules. As one representative example, the substance may be a therapeutic agent that can increase the expression and/or activity of the IRF3 gene or gene product (or other IRF gene product including but not limited to IRF7), and which thereby increases an antibody titer to an antigen as compared to an antibody titer amount that would occur in the absence of treatment with the therapeutic agent. In some examples, an adjuvant is a suspension of minerals (alum, aluminum hydroxide, aluminum phosphate) on which antigen is adsorbed; or water-in-oil emulsion in which antigen solution is emulsified in oil (MF-59, Freund's incomplete adjuvant), sometimes with the inclusion of killed mycobacteria (Freund's complete adjuvant) to further enhance antigenicity (inhibits degradation of antigen and/or causes influx of macrophages).

Administration: To provide or give a subject one or more agents, such as an agent that increases IRF expression (e.g., IRF3 expression, IRF7 expression, etc.) and/or treats one or more symptoms associated with a condition/disorder or disease including but not limited to stroke, cancer, chronic fatigue syndrome, multiple sclerosis, and viral infection/immune response to antigen, by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

Agent: Any protein, nucleic acid molecule (including chemically modified nucleic acids), compound, antibody, small molecule, organic compound, inorganic compound, or other molecule of interest. Agent can include a therapeutic agent, a diagnostic agent or a pharmaceutical agent. A therapeutic or pharmaceutical agent is one that alone or together with an additional compound induces the desired response (such as inducing a therapeutic or prophylactic effect when administered to a subject, including treating a subject suffering stroke, cancer, chronic fatigue syndrome, multiple sclerosis, or viral infection). Discussed herein, an agent may be referred to as a modulatory agent.

In some examples, an agent can act directly or indirectly to alter the expression and/or activity of one or more of IRFs (e.g., IRF3, IRF7, etc.), TLRs (e.g., TLR2, TLR3, TLR4, TLR, 7, TLR9, etc.), TRIF, among others. In a particular example, a therapeutic agent significantly increases the expression and/or activity of IRF3 thereby treating one or more signs or symptoms associated with stroke, acute ischemic events, etc. An example of a therapeutic agent is one that can increase the expression and/or activity of the IRF3 gene or gene product (or other IRF gene product including but not limited to IRF7), for example as measured by a clinical response such as a decrease in one or more signs or symptoms associated with stroke, an improvement in a patient outcome following stroke, an increased window of opportunity (or therapeutic window) in which to provide time-dependent treatments (e.g., tissue plasminogen activator (tPA) and mechanical thrombectomy) to patients experiencing stroke. With regard to stroke, “Increasing a window of opportunity” refers to increasing a time frame between an onset of an ischemic event and a time when time-dependent treatments (e.g., thrombolytics including but not limited to tissue plasminogen activator (tPA) and mechanical thrombectomy) can no longer be as effectively used (or used at all) to treat a patient experiencing the stroke. With regard to stroke, “improving a patient outcome” refers to at least reducing an amount of one or more of neural damage (e.g., reducing a size of an area surrounding an ischemic event referred to as the penumbra) and/or reducing an amount of loss of normal body function in a stroke patient by providing a therapeutic agent as compared to a case where the therapeutic agent is not provided to the stroke patient. With regard to stroke, “neural damage” refers to neural cell death and/or disruption of established neural signaling pathways. With regard to stroke, “loss of normal body function” includes, but is not limited to paralysis, speech/language abnormalities, changes in behavior, memory impairment, vision impairment, sexual function impairment, and organ damage.

In some examples disclosed herein, a therapeutic agent is one that can increase the expression and/or activity of the IRF3 gene or gene product (or other IRF gene product including but not limited to IRF7), for example as measured by a clinical response such as an improved cancer patient outcome as compared with a preexisting state or compared with a state which would occur in the absence of treatment with the therapeutic agent. For example, improving a cancer patient outcome may include a remission of the cancer (e.g., decrease in number of cancer cells and/or reduction in tumor size), an elimination of the cancer in the patient, a reduction or elimination of cancer metastasis, an improvement in immune system function, etc.

In some examples discussed herein, a therapeutic agent is one that can increase the expression and/or activity of the IRF3 gene or gene product (or other IRF gene product including but not limited to IRF7), for example as measured by a clinical response such as an improvement in one or more symptoms for a patient experiencing chronic fatigue syndrome. For example, improving one or more symptoms for such a patient may include a reduction in fatigue experienced by the patient, an improvement in the patient's memory and/or concentration, alleviation of sore throat symptoms, reduction in lymph node size, reduction in experienced muscle and/or joint pain, reduction in frequency and/or intensity of headaches, improvement to sleep, reduction in extent of exhaustion experienced after physical or mental exercise, etc., as compared with a preexisting state or compared with a state which would otherwise occur in the absence of treatment with the therapeutic agent.

In some examples discussed herein, a therapeutic agent is one that can increase the expression and/or activity of the IRF3 gene or gene product (or other IRF gene product including but not limited to IRF7), for example as measured by a clinical response such as an improvement in one or more symptoms for a multiple sclerosis patient. For example, improving one or more symptoms for a multiple sclerosis patient may include reducing or delaying visual changes including but not limited to double vision or loss of vision, reduction or delay of progression of numbness, reduction or delay of progression of tingling or weakness, reduction or delay of progression of paralysis, reduction or delay of progression of vertigo or dizziness, reduction or delay of progression of erectile or other sexual dysfunction, reduction or delay of progression of pregnancy complications, reduction or delay of progression of incontinence, or conversely, urinary retention, reduction or delay of progression of muscle spasticity, reduction or delay of progression of muscle incoordination, reduction or delay of progression of tremor, reduction or delay of progression of painful involuntary muscle contractions, reduction or delay of progression of slurred speech, and reduction or delay of progression of fatigue, etc., as compared with a preexisting state or compared with a state which would occur in the absence of treatment with the therapeutic agent.

In some examples discussed herein, a therapeutic agent is one that can increase the expression and/or activity of the IRF3 gene or gene product (or other IRF gene product including but not limited to IRF7), for example as measured by a clinical response such as an improvement in one or more symptoms for a patient experiencing a viral infection or immune response to antigen (in the case of vaccination). As one representative example, a patient may be infected with the Chikungunya virus. In such an example, an improvement in one or more symptoms may relate to a reduction in fever and/or joint pain, a reduction in headache, a reduction in muscle pain, a reduction in joint swelling, a reduction in rash, etc., as compared with a preexisting state or compared with a state which would occur in the absence of treatment with the therapeutic agent. As another representative example, a clinical response may include a measured increase in an antibody titer, for example in response to an antibody titer test, as compared to an antibody titer amount that would occur in the absence of treatment with the therapeutic agent.

Alkoxy: A radical (or substituent) having the structure —O—R, where R is a substituted or unsubstituted alkyl. Methoxy (—OCH₃) is an exemplary alkoxy group. In a substituted alkoxy, R is alkyl substituted with a non-interfering substituent. “Thioalkoxy” refers to —S—R, where R is substituted or unsubstituted alkyl. “Haloalkyloxy” means a radical —OR where R is a haloalkyl.

Alkoxy carbonyl: A group of the formula —C(O)OR, where R may be an optionally substituted alkyl or optionally substituted aryl. “Lower alkoxy carbonyl” groups are those where R contains from 1 to 10 (such as from 1 to 6) carbon atoms.

Alkyl: An acyclic, saturated, branched- or straight-chain hydrocarbon radical, which, unless expressly stated otherwise, contains from one to fifteen carbon atoms; for example, from one to ten, from one to six, or from one to four carbon atoms. This term includes, for example, groups such as methyl, ethyl, n-propyl, isopropyl, isobutyl, t-butyl, pentyl, heptyl, octyl, nonyl, decyl, or dodecyl. The term “lower alkyl” refers to an alkyl group containing from one to ten carbon atoms. Unless expressly referred to as an “unsubstituted alkyl,” alkyl groups can either be unsubstituted or substituted. An alkyl group can be substituted with one or more substituents (for example, up to two substituents for each methylene carbon in an alkyl chain). Exemplary alkyl substituents include, for instance, amino groups, amide, sulfonamide, halogen, cyano, carboxy, hydroxy, mercapto, trifluoromethyl, alkyl, alkoxy (such as methoxy), alkylthio, thioalkoxy, arylalkyl, heteroaryl, alkylamino, dialkylamino, alkylsulfano, keto, or other functionality.

Amino carbonyl (carbamoyl): A group of the formula —OCN(R)R′—, wherein R and R′ are independently of each other hydrogen or a lower alkyl group.

Analog or Derivative: A compound which is sufficiently homologous to a compound such that it has a similar functional activity for a desired purpose as the original compound. Analog or derivative refers to a form of a substance, such as ND-13 (refer to Table 8), which has at least one functional group altered, added, or removed, compared with a parent compound (e.g., ND-1, refer to Table 8). “Functional group” refers to a radical, other than a hydrocarbon radical, that adds a physical or chemical property to a substance.

AV-C: An interferon-activating molecule with a compound name of 1-(2-fluorophenyl)-2-(5-isopropyl-1,3,4-thiadiazol-2-yl)-1,2-dihydrochromeno[2,3-c]pyrrole-3,9-dione. Treatment of human cells with AV-C activates innate and interferon-associated responses that inhibit replication of Zika, Chikungunya and dengue viruses. AV-C has been shown to involve a TRIF-dependent signalling cascade that culminates in IFN regulatory factor 3 (IRF3)-dependent expression and secretion of type 1 interferon to elicit antiviral responses. It has further been shown that in response to AV-C, primary human peripheral blood mononuclear cells secrete pro-inflammatory cytokines that are linked with establishment of adaptive immunity to viral pathogens.

Biological activity: The beneficial or adverse effects of an agent on living matter. When the agent is a complex chemical mixture, this activity is exerted by the substance's active ingredient or pharmacophore, but can be modified by the other constituents. Activity is generally dosage-dependent and it is not uncommon to have effects ranging from beneficial to adverse for one substance when going from low to high doses. In one example, the agent significantly increases the biological activity of IRF3 (or other or other IRF gene products including but not limited to IRF7), cytokines, chemokines, etc., which reduces or delays one or more signs or symptoms associated with stroke.

BMDMs (bone marrow-derived macrophages): Primary macrophages obtained by in vitro differentiation of bone marrow cells in the presence of macrophage colony-stimulating factor (M-CSF or CSF1). They are readily obtainable in high yields, can be stored by freezing, and can be obtained from genetically modified mice strains.

BV2 cells: Cells derived from raf/myc-immortalized murine neonatal microglia. BV-2 cells have been used for pharmacological studies, studies of phagocytosis, neurodegeneration studies, and for many immunological discoveries.

Cancer: A physiological condition in mammals in which a population of cells are characterized by unregulated cell growth. Examples of cancer include, but are not limited to, Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kidney Cancer, Klatskin tumor, Krukenberg tumor, Laryngeal Cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple myeloma, Mycosis Fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neurofibroma, Neuroma, Nodular melanoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral cancer, Oropharyngeal Cancer, Osteosarcoma, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, and Wilms' tumor.

Neoplasia, malignancy, cancer and tumor may be used interchangeably and refer to abnormal growth of a tissue or cells that results from excessive cell division. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

Symptoms of cancer may include but are not limited to persistent cough or blood-tinged saliva, a change in bowel habits, blood in the stool, unexplained anemia (low blood count), breast lump or breast discharge, lumps in testicles, a change in urination, blood in urine, hoarseness, persistent lumps or swollen glands, obvious change of a wart or mole, indigestion, difficulty swallowing, unusual vaginal bleeding or discharge, unexpected weight loss, night sweats, or fever, continued itching in the anal or genital area, nonhealing sores, headaches, back pain, pelvic pain, and bloating, among others.

Carbamate: A group of the formula —OC(O)N(R)—, wherein R is H, or an aliphatic group, such as a lower alkyl group or an aralkyl group.

CC50 (50% cytotoxic concentration): A concentration of a compound required for the reduction of cell viability by 50%.

CHIKV (Chikungunya virus): A virus spread to humans by the bite of an infected mosquito. The most common symptoms of infection are fever and joint pain. Other symptoms may include headache, muscle pain, joint swelling, or rash. Symptoms generally begin 3-7 days after being bitten by an infected mosquito. People at risk for more severe disease include newborns infected around the time of birth, older adults (≥65 years), and people with medical conditions such as high blood pressure, diabetes, or heart disease. There is currently no vaccine to prevent or medicine to treat chikungunya virus.

Chemokine (Chemotactic cytokines): Small heparin-binding proteins that constitute a large family of peptides (60-100 amino acids) structurally related to cytokines, whose main function is to regulate cell trafficking. Chemokines can be classified into four subfamilies on the basis of the number and location of the cysteine residues at the N-terminus of the molecule and are named CXC, CC, CX₃C, and C. Chemokines are secreted in response to signals such as proinflammatory cytokines where they play an important role in selectively recruiting, for example, monocytes, neutrophils, and lymphocytes. Once induced, the directed migration of cells expressing the appropriate chemokine receptors occurs along a chemical ligand gradient known as the chemokine gradient. This allows cells to move toward high local concentrations of chemokines. The structure of chemokines comprise three distinct domains: (1) a highly flexible N-terminal domain, which is constrained by disulfide bonding between the N-terminal cysteine(s); (2) a long loop that leads into three antiparallel β-pleated sheets; and (3) an α-helix that overlies the sheets. Structure-function studies have revealed that the N-terminal region is important for receptor binding and activation. The majority of the chemokine ligands have a molecular mass between 8 kDa and 12 kDa and contain 1-3 disulfide bonds.

Chronic fatigue syndrome (CFS): A disorder characterized by extreme fatigue or tiredness that does not cease with rest and cannot be explained by an underlying medical condition. Also referred to as myalgic encephalomyelitis (ME) or systemic exertion intolerance disease (SEID).

Symptoms associated with CFS can include fatigue severe enough to interfere with daily activities, extreme fatigue after physical or mental exercises, feeling unrefreshed after a night's sleep, chronic insomnia, other sleep disorders, loss of memory, reduced concentration, orthostatic intolerance, muscle pain, frequent headaches, multi-joint pain without redness or swelling, frequent sore throat, tender and swollen lymph nodes, among others.

Clinical outcome: Refers to the health status of a patient following treatment for a disease or disorder, or in the absence of treatment. Clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy.

Contacting: Placement in direct physical association, including both a solid and liquid form. Contacting an agent with a cell can occur in vitro by adding the agent to isolated cells or in vivo by administering the agent to a subject.

Control: A sample or standard used for comparison with a test sample, such as a biological sample obtained from a patient (or plurality of patients) without a particular disease or condition, such as a patient or patients not having experienced stroke. In some embodiments, the control is a sample obtained from a healthy patient (or plurality of patients) (also referred to herein as a “normal” control), such as a normal biological sample. In some embodiments, the control is a historical control or standard value (e.g., a previously tested control sample or group of samples that represent baseline or normal values (e.g., expression values), such as baseline or normal values of a particular gene (such as an IRF gene) or gene product in a subject having not received a particular therapeutic agent (e.g., a therapeutic agent of the NA or ND chemofamilies). A control represents an untreated sample (e.g., absence of therapeutic agent) for comparison with a treated sample (e.g., treated with the therapeutic agent).

CpG (cytosine-phosphate-guanine oligodeoxynucleotide): A Toll-like receptor agonist which is known to reduce ischemic injury in both rodent and nonhuman primate models of experimental stroke.

Cytokine: A term for a diverse group of soluble proteins and peptides released from cells which act as humoral regulators at nano- to picomolar concentrations, and which, either under normal or pathological conditions, modulate the functional activities of individual cells and tissues. These proteins also mediate interactions between cells directly and regulate processes taking place in the extracellular environment. Many growth factors and cytokines act as cellular survival factors by preventing programmed cell death. Cytokines include both naturally occurring peptides and variants that retain full or partial biological activity.

CXCL10 (chemokine(C-X-C) motif ligand 10): A pro-inflammatory cytokine that is involved in a wide variety of processes such as chemotaxis, differentiation, and activation of peripheral immune cells, regulation of cell growth, apoptosis and modulation of angiostatic effects. Plays thereby an important role during viral infections by stimulating the activation and migration of immune cells to the infected sites. CXCL10 plays a pleiotropic role in prolonged leukocyte recruitment, astrocyte migration/activation, and neural attachment/sprouting following focal stroke. CXCL10 is associated with ischemic stroke independent of traditional cardiovascular risk factors.

CXCL10 sequences are publicly available on GenBank. See, for example, Gene Accession number NM_001565.4 (human) and NM_021274.2 (mouse), each of which is herein incorporated by reference as available on May 4, 2020. Decrease: To reduce the quality, amount, or strength of something. In one example, a therapy (e.g., administration of a therapeutic agent of the present disclosure) decreases one or more symptoms associated with stroke, for example as compared to the response in the absence of the therapy.

Derivative: A chemical substance that differs from another chemical substance by one or more functional groups. Preferably, a derivative retains a biological activity of a molecule from which it was derived.

Diagnosis: The process of identifying a disease, such as muscular dystrophy, by its signs, symptoms and results of various tests. The conclusion reached through that process is also called “a diagnosis.” Forms of testing commonly performed include blood tests, medical imaging, urinalysis, and biopsy.

DMXAA: A compound with the name 5,6-dimethylxanthenone-4-acetic acid, also known as Vadimezan. DMXAA is a mouse-specific STING (stimulator of interferon genes) agonist and activator of interferon regulatory factor (IRF). STING is a ubiquitous intracellular protein activated by cyclic dinucleotides that are generated from cytosolic DNA coming from DNA viruses or dying cells. The activation of STING leads mainly to the activation of TBK1 (TANK-binding kinase-1)/IRF3 and the downstream transcription of type I interferon genes. DMXAA was initially identified as a potent tumor vascular disrupting agent in mice. The antitumor activity of DMXAA has been linked to its ability to induce a variety of cytokines and chemokines, including TNF-α, IP-10, IL-6 and RANTES.

EC50: A concentration of a drug that gives a half-maximal response.

Effective amount: An amount of agent that is sufficient to generate a desired response, such as reducing or inhibiting one or more signs or symptoms associated with a condition or disease. When administered to a subject, a dosage will generally be used that will achieve target tissue/cell concentrations. In some examples, an “effective amount” is one that treats one or more symptoms and/or underlying causes of any of a disorder or disease. In a representative example, an “effective amount” is a therapeutically effective amount in which the agent alone or with an additional therapeutic agent(s) (for example anti-thrombolytic agents in the context of stroke), induces the desired response such as reduction in one or more symptoms associated with stroke.

In particular examples, it is an amount of an agent capable of increasing IRF3 gene expression or activity (or other IRF including but not limited to IRF7) by least 10%, at least 20%, at least 50%, at least 60%, at least 70%, at least 80%, at least 90%, at least 95%, at least 98%, or even at least 100%.

In some examples, an effective amount is an amount of a pharmaceutical preparation that alone, or together with a pharmaceutically acceptable carrier or one or more additional therapeutic agents, induces the desired response.

In one example, a desired response is to increase the subject's survival time and/or improve the subject's quality of life, for example by reducing a number and/or amount of symptoms associated with a stroke. In another example, a desired response is to increase the subject's survival time and/or improve the subject's quality of life by slowing or eliminating progression of disease, such as slowing or eliminating the progression of cancer.

The symptoms and/or underlying cause of a disease, syndrome, viral infection, etc., do not need to be completely inhibited for the pharmaceutical preparation to be effective. For example, a pharmaceutical preparation may decrease the progression of the disease, syndrome, viral infection, etc., by a desired amount, for example by at least 10%, at least 20%, at least 50%, at least 60%, at least 70%, at least 80%, at least 90%, at least 95%, at least 98%, or even at least 100%, as compared to the progression typical in the absence of the pharmaceutical preparation.

In another or additional example, it is an amount sufficient to partially or completely alleviate symptoms of the disease (e.g., stroke) within the subject. Treatment can involve only slowing the progression of the disease temporarily, but can also include halting or reversing the progression of the disease permanently.

Effective amounts of the agents described herein can be determined in many different ways, such as, for example, assaying for a reduction in of one or more signs or symptoms associated with a stroke or ischemic event in the subject or measuring the expression level of one or more molecules known to be associated with stroke. Effective amounts also can be determined through various in vitro, in vivo or in situ assays, including the assays described herein.

The disclosed therapeutic agents can be administered in a single dose, or in several doses, for example hourly, daily, weekly, monthly, yearly, during a course of treatment. The effective amount can be dependent on the subject being treated, the severity and type of the condition being treated, and the manner of administration.

Expression: The process by which the coded information of a gene is converted into an operational, non-operational, or structural part of a cell, such as the synthesis of a protein. Gene expression can be influenced by external signals. For instance, exposure of a cell to a hormone may stimulate expression of a hormone-induced gene. Different types of cells can respond differently to an identical signal. Expression of a gene also can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced. In an example, expression, such as expression of IRF3 (or other IRF including but not limited to IRF7), can be regulated to treat one or more signs or symptoms associated with stroke, cancer, chronic fatigue syndrome, multiple sclerosis, viral infection, etc., as discussed herein.

The expression of a nucleic acid molecule can be altered relative to a normal (wild type) nucleic acid molecule. Alterations in gene expression, such as differential expression, include but are not limited to: (1) overexpression; (2) underexpression; or (3) suppression of expression. Alterations in the expression of a nucleic acid molecule can be associated with, and in fact cause, a change in expression of the corresponding protein.

Protein expression can also be altered in some manner to be different from the expression of the protein in a normal (wild type) situation. This includes but is not necessarily limited to: (1) a mutation in the protein such that one or more of the amino acid residues is different; (2) a short deletion or addition of one or a few (such as no more than 10-20) amino acid residues to the sequence of the protein; (3) a longer deletion or addition of amino acid residues (such as at least 20 residues), such that an entire protein domain or sub-domain is removed or added; (4) expression of an increased amount of the protein compared to a control or standard amount; (5) expression of a decreased amount of the protein compared to a control or standard amount; (6) alteration of the subcellular localization or targeting of the protein; (7) alteration of the temporally regulated expression of the protein (such that the protein is expressed when it normally would not be, or alternatively is not expressed when it nominally would be); (8) alteration in stability of a protein through increased longevity in the time that the protein remains localized in a cell; and (9) alteration of the localized (such as organ or tissue specific or subcellular localization) expression of the protein (such that the protein is not expressed where it would normally be expressed or is expressed where it normally would not be expressed), each compared to a control or standard.

Gene knockout (KO): Refers to a genetic technique in which one of an organism's or cell's genes is made inoperative. Knockout can also refer to the particular gene that is knocked out or to the organism or cell line that carries the gene knock-out. A knockout mouse is a genetically modified mouse (Mus Musculus) in which an existing gene is knocked out by replacing it, deleting it, or otherwise disrupting it (e.g., with an artificial piece of DNA).

Gro-alpha (chemokine (C-X-C) ligand 1): Growth-regulated oncogene-alpha is a member of the CXC family and potent neutrophil chemoattractant, which plays an integral role in recruitment and activation of neutrophils in response to tissue injury and microbial infection. Inflammation is known to accompany and exacerbate cerebral ischemia, and the infiltrated leucocytes are thought to contribute to tissue injury in stroke patients. There is evidence that supports a role for Gro-alpha in the inflammatory reaction that occurs during an early phase of ischemic stroke.

Gro-alpha sequences are publicly available on GenBank. See, for example, Gene Accession number NM_001511.4 (human) and NM_008176.3 (mouse), each of which is herein incorporated by reference as available on May 4, 2020.

High-throughput screening: A method for scientific experimentation, for example in relation to drug discovery, in which a large number (e.g., hundreds to millions) of chemical, genetic, or pharmacological tests can be rapidly conducted using one or more of robotics, data processing/control software, liquid handling devices, sensitive detectors, etc. The process can enable rapid identification of compounds, antibodies, or genes that modulate a particular biochemical pathway.

IFIT1 (interferon-induced protein with tetratricopeptide repeats 1): The IFIT1 gene encodes a protein containing tetratricopeptide repeats that was originally identified as induced upon treatment with interferon. The encoded protein may inhibit viral replication and translational initiation.

IFIT1 sequences are publicly available on GenBank. See, for example, Gene Accession number NM_001548.5 (human) and NM_008331.3 (mouse), each of which is herein incorporated by reference as available on May 4, 2020.

IFIT3 (interferon-induced protein with tetratricopeptide repeats 3): The IFIT3 gene encodes an interferon-induced antiviral protein which acts as an inhibitor of cellular as well as viral processes, cell migration, proliferation, signaling, and viral replication.

IFIT3 sequences are publicly available on GenBank. See, for example, Gene Accession number NM_001549.6 (human) and NM_010501.2 (mouse), each of which is herein incorporated by reference as available on May 4, 2020.

IL-6 (interleukin-6): IL6 is a pro-inflammatory cytokine secreted by T cells and macrophages that influences antigen-specific immune responses and inflammatory reactions. IL-6 sequences are publicly available. For example, GENBANK® Accession number AF372214.2 (deposited Jun. 1, 2001) discloses a human IL-6 gene sequence, and GENBANK® Accession numbers BC015511.1 and AAH15511.1 (each deposited Oct. 4, 2001) disclose human IL-6 mRNA and pre-protein sequences, respectively. One skilled in the art will appreciate that IL-6 nucleic acid and protein molecules can vary from those publicly available, such as those polymorphisms resulting in one or more substitutions, deletions, insertions, or combinations thereof, while still retaining IL-6 biological activity (e.g., increased expression in colon adenocarcinoma).

Increase or upregulate: To enhance the quality, amount, or strength of something. In one example, an agent increases the activity or expression of IRF3 (or other IRF including but not limited to IRF7), for example relative to an absence of the agent. In a particular example, an agent increases the activity or expression of IRF3 by at least 10%, at least 20%, at least 50%, or even at least 90%, including between 10% to 95%, 20% to 80%, 30% to 70%, 40% to 50%, such as 10%, 20%, 30%, 40%, 50%, 60%, 70%, 80%, 85%, 90%, 95%, 98%, or 100%. Such increases can be measured using the methods disclosed herein.

In a particular example, a therapy increases (also known as up-regulates) the expression of IRF3, such as an increase of at least 10%, at least 20%, at least 50%, or even at least 90% in IRF3 expression, thereby treating/alleviating one or more signs or symptoms associated with, for example, stroke. In some examples, an increase in expression refers to an increase in an IRF3 gene product or activity of an IRF3 gene product. An IRF3 gene product can be RNA (such as mRNA, rRNA, tRNA, and structural RNA) or protein.

Gene upregulation includes any detectable increase in the production of a gene product (e.g., IRF3 gene product, IRF7 gene product, etc.). In certain examples, production of an IRF3 gene product increases by at least 2-fold, for example at least 3-fold or at least 4-fold responsive to administration of an agent, as compared to a control (such an amount of gene expression in a cell that has not been exposed to the agent). In one example, a control is a relative amount of IRF3 gene expression or protein expression in a biological sample taken from a subject that has not received a therapeutic agent, such as an agent selected from the chemofamilies ND and NA as disclosed herein. Such increases can be measured using the methods disclosed herein. For example, “detecting or measuring expression of IRF3” includes quantifying the amount of the gene, gene product or modulator thereof present in a sample. Quantification can be either numerical or relative. Detecting expression of the gene, gene product or modulators thereof can be achieved using any method known in the art or described herein, such as by measuring nucleic acids by PCR (such as quantitative RT-PCR) and proteins by ELISA, or reporter constructs. In primary embodiments, the change detected is an increase or decrease in expression as compared to a control, such as a biological sample or subject that has not been exposed or contacted with a therapeutic agent (e.g., a therapeutic agent of the NA or ND chemofamilies). In some examples, the detected increase or decrease is an increase or decrease of at least two-fold compared with the control or standard.

In other embodiments of the methods, the increase or decrease is of a diagnostically significant amount, which refers to a change of a sufficient magnitude to provide a desired response.

The level of expression in either a qualitative or quantitative manner can include detection of nucleic acid or protein. Exemplary methods include microarray analysis, RT-PCR, Northern blot, Western blot, and mass spectrometry.

Inflammatory gene: Refers to any gene that influences inflammatory responses or inflammatory signaling pathways. Inflammatory genes include, but are not limited to, proinflammatory cytokines, transcription factors that regulate inflammatory responses and immune cell signaling molecules.

Inhibiting a disease or condition: A phrase referring to reducing the development of a disease or condition, for example, in a subject who is at risk for a disease or who has a particular disease. Particular methods of the present disclosure provide methods for inhibiting neuronal cell death in response to stroke, as a representative example.

Interferons: A family of more than 15 related proteins with three major classes (α, β, and γ). Viruses are the prototypic inducer of interferon production. Interferons are pleiotropic cellular modulators which induce expression of a wide variety of genes including, but not limited to, chemokines, adhesion proteins, intracellular enzymes, and transcription factors.

IP (ischemic preconditioning): A brief ischemia that does not cause injury to the ischemic organ and which prevents or reduces ischemic injury caused by a subsequent, prolonged ischemia. Ischemic preconditioning can be implemented by middle cerebral artery occlusion (MCAO), for example, as discussed in greater detail below.

IP-10: See above description for CXCL10. CXCL10 is also referred to as interferon-γ-inducible protein 10.

IPS1 (interferon-β promotor stimulator 1): IPS1 has been shown to interact with several signaling proteins, such as tumor necrosis factor receptor-associated factor 2 (TRAF2), TRAF6, Fas-associated protein with the death domain (FADD), and receptor interacting protein-1 (RIP1). These molecules presumably participate in NF-κB activation and proinflammatory cytokine induction. IPS-1 also interacts with TRAF3, which is required for the activation of kinases for interferon regulatory factors (IRFs) to induce type I IFN gene induction. IPS1 is also referred to as mitochondrial antiviral signaling (MAVS), CARD adaptor inducing IFN-β (Cardif), and virus-induced signaling adaptor (VISA).

IPS-1 sequences are publicly available on GenBank. See, for example, Gene Accession number AB232371.1 (human) and NM_144888.2 (mouse), each of which is herein incorporated by reference as available on May 4, 2020.

IRF proteins: Members of the interferon-regulatory factor (IRF) protein family were originally identified as transcriptional regulators of the Type I interferon system. Several IRFs are known to be critical for the elicitation of innate pattern recognition receptors, and thus for adaptive immunity. IRFs may be involved in modulating cellular responses that are involved in tumorigenesis, and may be involved in cardiovascular diseases, arising from their participation in divergent and overlapping molecular programs beyond the immune response.

All IRF family members possess an N-terminal DNA-binding domain (DBD) that is characterized by a series of five relatively well-conserved tryptophan-rich repeats. The DBD forms a helix-turn-helix structure and recognizes a DNA sequence known as IFN-stimulated response element (ISRE). The C-terminal region of IRFs is less well conserved and has been proposed to mediate the interactions of a specific IRF with other family members, other transcriptional factors, or cofactors, so as to confer specific activities upon each IRF. The nature of protein-to-protein interactions dictated by C-terminal domains is proposed to determine whether the resulting complex functions as a transcriptional activator or repressor, and to define the nucleotide sequences adjacent to the core IRF-binding motif to which the transcriptional complex binds.

IRF3 is expressed in most cell types and organs. Diseases related to IRF3 dysfunction include but are not limited to dilated cardiomyopathy, hypertrophic cardiomyopathy, stroke, Ankylosing spondylitis (AS), and Abdominal aortic aneurysm (AAA). IRF3 functions include, but are not limited to, induction of type I IFNs, reduction of cardiac hypertrophy, as elements for TLR ligands pretreatment-induced tolerance to stroke, and inhibition of neointimal formation, among others.

IRF7 is expressed in most cell types and organs, similarly to IRF3. Diseases related to IRF7 dysfunction include but are not limited to influenza infection, systemic lupus erythematosus (SLE), heart failure, stroke, AS, and AAA. IRF7 functions include but are not limited to positive regulation of TLR-dependent proinflammatory responses, suppression of cardiac remodeling, involved in TLR ligands pretreatment-elicited neuroprotection, alleviation of ischemic stroke, and inhibition of neointimal formation, among others.

IRF3 sequences are publicly available on GenBank. See, for example, Gene Accession number NM_001571.6 (human) and NM_016849.4 (mouse), each of which is herein incorporated by reference as available on May 4, 2020.

IRF7 sequences are publicly available on GenBank. See, for example, Gene Accession number NM_001572.5 (human) and NM_001252600.1 (mouse), each of which is herein incorporated by reference as available on May 4, 2020.

IRF-selective compounds: Compounds defined as meeting a criteria of >3 σ for ISRE activation and <1σ for NFκB activation as monitored via an NFκB-SEAP (secreted embryonic alkaline phosphatase) and IRF-Lucia luciferase dual reporter featuring two reporter genes SEAP and Lucia luciferase thereby enabling the simultaneous study of the NF-κB and IRF signaling pathways. The Lucia luciferase reporter gene is under the control of an ISG54 (interferon-stimulated gene) minimal promoter in conjunction with five IFN-stimulated response elements (ISREs). The SEAP gene is driven by an IFN-β minimal promoter fused to five copies of the NF-κB consensus transcriptional response element and three copies of the c-Rel binding site. Both reporter proteins are readily measurable in the cell culture supernatant when using QUANTI-Luc™ (InvivoGen) and QUANTI-Blue (InvivoGen) SEAP detection reagents.

ISG15 (interferon-stimulated gene 15): The Ubiquitin-like ISG-15 gene is implicated as a central player in host antiviral response. ISG15 can be covalently conjugated onto target proteins via an enzymatic cascade. ISG15 and the members of the enzymatic cascade that mediate ISG15 conjugation (ISGylation) are strongly induced by type I interferons.

ISG15 sequences are publicly available on GenBank. See, for example, Gene Accession number NM_005101.4 (human) and NM_015783.3 (mouse), each of which is herein incorporated by reference as available on May 4, 2020.

Isolated: An “isolated” biological component (such as a nucleic acid molecule, protein, or cell) has been substantially separated or purified away from other biological components in the cell of the organism, or the organism itself, in which the component naturally occurs, such as other chromosomal and extra-chromosomal DNA and RNA, proteins and cells. Nucleic acid molecules and proteins that have been “isolated” may be understood to have been purified by standard purification methods. The term also embraces nucleic acid molecules and proteins prepared by recombinant expression in a host cell as well as chemically synthesized nucleic acid molecules and proteins.

Isomers: Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center and is described by the R and S sequencing rules of Cahn and Prelog, or by the manner in which the molecule rotates the plane of polarized light and designated as dextrorotatory or levorotatory (i.e., as (+) or ( ) isomers, respectively). A chiral compound can exist as either an individual enantiomer or as a mixture thereof. A mixture containing equal proportions of the enantiomers is called a “racemic mixture.” Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see, e.g., March, Advanced Organic Chemistry, 4th edition, New York: John Wiley and Sons, 1992, Chapter 4).

ISRE (interferon stimulated response element): DNA binding elements in certain gene promoters that serve as the binding site for a family of transcription factors termed interferon regulatory factors (IRFs).

LPS (lipopolysaccharide) preconditioning: Induction of neuroprotection against stroke via a dose of LPS given systematically prior to a stroke. There is evidence to suggest that LPS preconditioning leads to decreased cellular infiltration into the injured ischemic brain and suppressed cellular activation, suggestive that LPS preconditioning alters cellular responsiveness to subsequent injurious stimuli.

Lucia luciferase: A secreted luciferase expressed by a synthetic gene designed on the naturally secreted luciferases from marine copepods. Lucia luciferase has been engineered for superior properties compared to natural secreted luciferases.

The superior bioluminescence signal generated by Lucia luciferase is magnitudes stronger than the commonly used firefly and Renilla luciferases. The intense bioluminescence facilitates real-time measurements to detect very small amounts of the reporter in the cell culture medium and slight changes in the reporter concentration. Furthermore, the Lucia luciferase gene is codon optimized and free of CpG dinucleotides for prolonged mammalian cell expression.

Luciferase: A generic term for a class of oxidative enzymes that produce bioluminescence. Found naturally in insect fireflies and in luminous marine and terrestrial microorganisms, luciferase is thus a light-producing enzyme. When expressed in mammalian or insect cells, the native signal sequences of these luciferases are functionally active, mediating their export from within the cell to the surrounding culture medium. Bioluminescence assays are conducted using culture media, whereupon the activity of the secreted luciferases provides a readout of the biological signaling event under study.

MCAO (middle cerebral artery occlusion): A model of stroke involving the insertion of a surgical filament into the external carotid artery followed by threading it forward into the internal carotid artery (ICA) until the tip occludes the origin of the middle cerebral artery (MCA), resulting in a cessation of blood flow and subsequent brain infarction in the MCA territory.

MCP1 (monocyte chemoattractant protein-1): A chemokine that regulates migration and infiltration of monocytes/macrophages. MCP1 is a member of the C-C chemokine family, and is also referred to as CCL2. MCP1 is considered IFN-regulated or IFN-stimulated because is it partially controlled by an upstream ISRE, although other response elements are present in the MCP1 promoter region.

MCP1 sequences are publicly available on GenBank. See, for example, Gene Accession number NM_002982.4 (human) and NM_011333.3 (mouse), each of which is herein incorporated by reference as available on May 4, 2020.

MCP3 (monocyte chemoattractant protein-1): A chemokine that is chemotactic for and activates a variety of inflammatory cell types. MCP3 is a member of the C-C chemokine family, and is also referred to as CCL7. MCP3 is structurally related to MCP1, but with a distinct receptor usage and spectrum of action.

MCP3 sequences are publicly available on GenBank. See, for example, Gene Accession number NM_006273.4 (human) and NM_013654.3 (mouse), each of which is herein incorporated by reference as available on May 4, 2020.

MIP1a (macrophage inflammatory protein 1 alpha): A member of the CC chemokine subfamily that was originally purified from conditioned media of an LPS-stimulated murine macrophage cell line. MIP1a is also referred to as CCL3. MIP1a is produced by macrophages and acts as a chemoattractant to a variety of cells including monocytes, T cells, B cells and eosinophils. MIP1a is considered IFN-regulated or IFN-stimulated because it is partially controlled by its upstream ISRE, although other promoter sites are present. There is evidence in human fibroblast-like synoviocytes (FLS) that IRF3 deficiency markedly reduces MIP1a gene expression.

MIP1a sequences are publicly available on GenBank. See, for example, Gene Accession number NM_002983.3 (human) and NM_011337.2 (mouse), each of which is herein incorporated by reference as available on May 4, 2020.

MIP1b (macrophage inflammatory protein 1 beta): A member of the CC chemokine subfamily, and which is also referred to as CCL4. MIP1b is produced by macrophages, and activates granulocytes (neutrophils, eosinophils and basophils) which can lead to acute neutrophilic inflammation.

MIP1b sequences are publicly available on GenBank. See, for example, Gene Accession number NM_002984.4 (human) and NM_013652.2 (mouse), each of which is herein incorporated by reference as available on May 4, 2020.

Multiple Sclerosis (MS): A potentially disabling disease of the brain and spinal cord (central nervous system). In MS, the immune system attacks the protective sheath (myelin) that covers nerve fibers and causes communication problems between the brain and the rest of the body. Eventually, the disease can cause permanent damage or deterioration of the nerves.

Multiple sclerosis signs and symptoms may differ greatly from person to person and over the course of the disease depending on the location of affected nerve fibers. Symptoms often affect movement, and may include numbness or weakness in one or more limbs that typically occurs on one side of the body at a time (or the legs and trunk), electric-shock sensations that occur with certain neck movements, especially bending the neck forward (Lhermitte sign) and/or tremor, lack of coordination or unsteady gait. Vision problems are also common, including but not limited to partial or complete loss of vision (usually in one eye at a time) often with pain during eye movement, prolonged double vision, and/or blurry vision. Other symptoms may include slurred speech, fatigue, dizziness, tingling or pain in parts of the body, problems with sexual, bowel and bladder function.

The cause of MS is unknown. It is considered an autoimmune disease in which the body's immune system attacks its own tissues. In the case of MS, this immune system malfunction destroys the fatty substance that coats and protects nerve fibers in the brain and spinal cord (myelin).

MS patients may also develop other complications, including but not limited to muscle stiffness or spasms, paralysis (typically in the legs), problems with bladder, bowel or sexual function, mental changes (e.g., forgetfulness or mood swings), depression, and epilepsy.

Neuro2A cells: Cell line with an origin of mouse albino neuroblastoma. The cell line was derived from a spontaneous tumor in an albino strain A mouse. Cells produce microtubular protein which is believed to play a role in the contractile system giving axoplasmic flow in nerve cells.

NF-κB (nuclear factor kappa-light-chain-enhancer of activated B cells): A protein complex that controls transcription of DNA, cytokine production, and cell survival. NF-κB is found in almost all animal cell types and is involved in cellular responses to stimuli such as stress, cytokines, free radicals, heavy metals, ultraviolet irradiation, oxidized LDL, and bacterial or viral antigens.

Oasl2 (2′-5′-oligoadenylate synthase-like protein 2): An interferon-induced dsRNA-activated antiviral enzyme which plays a critical role in cellular innate antiviral response. There is evidence in studies with adipocytes that IRF3 can regulate expression of Oasl2. There is further evidence showing that Oasl2 is regulated differentially after 24 hours post stroke onset in mice preconditioned with LPS, CpG or brief ishemia, as compared to mice that did not receive any preconditioning.

Oasl2 sequences are publicly available on GenBank. See, for example, Gene Accession number NM_011854.2 (mouse) which is herein incorporated by reference as available on May 4, 2020.

OGD (oxygen glucose deprivation): A method for inducing ischemic injury in vitro. In practice, this treatment consists of replacement of a standard culture medium with a hypoxic N₂/CO₂ equilibrated culture medium without glucose, and the incubation of the cells in a hypoxic chamber having the same N₂/CO₂ gas combination.

Optional: “Optional” or “optionally” means that the subsequently described event or circumstance can but need not occur, and that the description includes instances where said event or circumstance occurs and instances where it does not.

Patient: As used herein, the term “patient” includes human and non-human animals. The preferred patient for treatment is a human. “Patient” and “subject” are used interchangeably herein.

Pharmaceutically acceptable carriers: The pharmaceutically acceptable carriers (vehicles) useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of one or more agents, such as one or more 001 modulatory agents.

In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical agents to be administered can contain minor amounts of non-toxic auxiliary substances, such as wetting or emulsifying agents, preservatives, and pH buffering agents and the like, for example sodium acetate or sorbitan monolaurate, sodium lactate, potassium chloride, calcium chloride, and triethanolamine oleate.

Phenyl: Phenyl groups may be unsubstituted or substituted with one, two or three substituents, with substituent(s) independently selected from alkyl, heteroalkyl, aliphatic, heteroaliphatic, thioalkoxy, halo, haloalkyl (such as CF3), nitro, cyano, OR (where R is hydrogen or alkyl), N(R)R′ (where R and R′ are independently of each other hydrogen or alkyl), COOR (where R is hydrogen or alkyl) or —C(O)N(R′)R″ (where R′ and R″ are independently selected from hydrogen or alkyl).

Poly ICLC: A synthetic complex of carboxymethylcellulose, polyinosinic-polycytidylic acid, and poly-L-lysine double stranded RNA. The brand name of poly ICLC in the United States is Hiltonol.

Preconditioning: A brief ischemic event that causes a complex reprogramming of cellular responses to a subsequent ischemia.

Preventing, treating or ameliorating a disease: “Preventing” a disease (such as stroke or cancer) refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Purified: The term “purified” does not require absolute purity; rather, it is intended as a relative term. Thus, for example, a purified protein preparation is one in which the protein referred to is more pure than the protein in its natural environment within a cell. For example, a preparation of a protein (such as an inflammatory protein) is purified such that the protein represents at least 50% of the total protein content of the preparation. Similarly, a purified oligonucleotide preparation is one in which the oligonucleotide is more pure than in an environment including a complex mixture of oligonucleotides. Purity of a compound may be determined, for example, by high performance liquid chromatography (HPLC) or other conventional methods. Compounds described herein may be obtained in a purified form or purified by any of the means known in the art, including silica gel and/or alumina chromatography. See, e.g., Introduction to Modern Liquid Chromatography, 2nd Edition, ed. by Snyder and Kirkland, New York: John Wiley and Sons, 1979; and Thin Layer Chromatography, ed. by Stahl, New York: Springer Verlag, 1969.

PYCARD (PYD and CARD domain containing): Functions as a key mediator in apoptosis and inflammation. PYCARD sequences are publicly available on GenBank. See, for example, Gene Accession number NM_013258.5 (human) and NM_023258.4 (mouse), each of which are herein incorporated by reference as available on May 4, 2020.

PYHIN1 (pyrin and HIN-domain family member 1): Belongs to the HIN200 family of interferon-inducible proteins that share a 200-amino acid signature motif at their C-terminal ends. HIN200 proteins are primarily nuclear and are involved in transcriptional regulation of genes important for cell cycle control, differentiation and apoptosis.

Pyhin1 sequences are publicly available on GenBank. See, for example, Gene Accession number NM_152501.5 (human) and NM_175026.3 (mouse), each of which are herein incorporated by reference as available on May 4, 2020.

RANTES (regulated upon activation, Normal T cell Expressed and Presumably Secreted): A chemotactic cytokine or chemokine also known as CCL5 that is chemotactic for T cells, eosinophils, and basophils, and which plays an active role in recruiting leukocytes into inflammatory sites. With the help of particular cytokines (IL-2 and IFN-gamma) that are released by T cells, Rantes induces the proliferation and activation of certain natural killer (NK) cells to form CC chemokine-activated killer cells.

RANTES sequences are publicly available on GenBank. See, for example, Gene Accession number NM_001278736.2 (human) and NM_013653.3 (mouse), each of which are herein incorporated by reference as available on May 4, 2020.

RSAD2 (radical S-adenosyl methionine domain containing 2): Plays a role in cell antiviral state induced by type I, II and III interferon. Also referred to as Viperin. RSAD2 can inhibit a wide range of DNA and RNA viruses, including human cytomegalovirus (HCMV), hepatitis C virus (HCV), west Nile virus (WNV), dengue virus, sindbis virus, influenza A virus, Sendai virus, vesicular stomatitis virus (VSV), and human immunodeficiency virus (HIV-1).

RSAD2 sequences are publicly available on GenBank. See, for example, Gene Accession number NM_080657.5 (human) and NM_021384.4 (mouse), each of which are herein incorporated by reference as available on May 4, 2020.

RTP4 (receptor transporting protein 4): Functions as a chaperone protein which facilitates trafficking and functional cell surface expression of some G-protein coupled receptors (GPCRs). RTP4 sequences are publicly available on GenBank. See, for example, Gene Accession number NM_022147.3 (human) and NM_023386.5 (mouse), each of which are herein incorporated by reference as available on May 4, 2020.

Sample (or biological sample): A biological specimen containing genomic DNA, RNA (including mRNA), protein, or combinations thereof, obtained from a subject. Examples include, but are not limited to, peripheral blood, urine, saliva, tissue biopsy, surgical specimen, and autopsy material. In one example, a sample includes tumor biopsy, such as from a subject with cancer.

SEAP (secreted embryonic alkaline phosphatase): As discussed herein, SEAP is a reporter widely used to study promoter activity or gene expression. It is a truncated form of human placental alkaline phosphatase (PLAP) by deletion of the GPI anchor.

Selectivity index: A ratio of ISRE:NFκB activity in response to agents (e.g., compounds of the NA chemofamily and/or ND chemofamily) of the present disclosure as monitored via the NFκB-SEAP and IRF-Lucia luciferase dual reporter.

Sendai: Replication-incompetent inactivated Sendai virus particles (Hemagglutinating virus of Japan envelope, HVJ-E). It has been shown that HVJ-E directly leads to apoptosis in a dose-dependent manner in human prostate cancer cell lines. Further analysis has shown that HJV-E-derived RNA fragments (˜300 bp in length) were responsible for cancer-selective apoptosis, as host-derived retinoic acid-inducible gene-I (RIG-1) recognizes these RNA fragments and interacts with the IFN-β promoter stimulator-I (IPS-1) to initiate a signaling cascade that activates type I IFN production.

SH-SY5Y: A thrice cloned human cell line derived from the SK-N-SH neuroblastoma cell line. It serves as a model for neurodegenerative disorders as the cells can be converted to various types of functional neurons by the addition of specific compounds. The parental SK-N-SH cell line was established from metastatic cells found in the bone marrow aspirate of a four-year-old female of unknown ethnicity.

Signs or symptoms: Any subjective evidence of disease or of a subject's condition, e.g., such evidence as perceived by the subject; a noticeable change in a subject's condition indicative of some bodily or mental state. A “sign” is any abnormality indicative of disease, discoverable on examination or assessment of a subject. A sign is generally an objective indication of disease. Signs include, but are not limited to any measurable parameters such as tests for detecting stroke, including face drooping, arm weakness, speech difficulty, measurable parameters related to stroke inferred based on computed tomography (CT) scans and/or magnetic resonance imaging (MRI) scans, measurable parameters related to stroke inferred based on blood tests, electrocardiogram (EKG), carotid ultrasound, echocardiographs, cerebral angiography, etc.

In one example, reducing or inhibiting one or more symptoms or signs associated with stroke includes increasing the activity or expression of IRF3 (or other IRF including but not limited to IRF7) by a desired amount, for example by at least 10%, at least 20%, at least 50%, at least 60%, at least 70%, at least 80%, at least 90%, at least 95%, at least 98%, or even at least 100%, as compared to the activity and/or expression in the absence of the treatment.

Stroke: Broadly refers to the sudden death of brain cells due to lack of oxygen, caused by a blockage of blood flow or rupture of an artery to the brain. There are three main types of stroke 1) ischemic stroke, 2) hemorrhagic stroke, and 3) transient ischemic attack. Most strokes (e.g., upwards of 85%) are ischemic strokes.

An ischemic stroke happens when blood flow through the artery that supplies oxygen-rich blood to the brain becomes blocked. Blood clots often cause the blockages that lead to ischemic strokes.

A hemorrhagic stroke happens when an artery in the brain leaks blood or ruptures (breaks open). The leaked blood puts too much pressure on brain cells, thereby damaging them. High blood pressure and aneurysms (balloon-like bulges in an artery that can stretch and burst) are examples of conditions that can cause a hemorrhagic stroke. There are two types of hemorrhagic strokes. Intracerebral hemorrhage is the most common type of hemorrhagic stroke. It occurs when an artery in the brain bursts, flooding the surrounding tissue with blood. Subarachnoid hemorrhage is a less common type of hemorrhagic stroke. It refers to bleeding in the area between the brain and the thin tissues that cover it.

A transient ischemic attack (TIA) is sometimes called a “mini-stroke.” It is different from the major types of stroke because blood flow to the brain is blocked for only a short time (usually no more than 5 minutes).

Symptoms of stroke include, but are not limited to difficulty walking, instability, paralysis with weak muscles, problems with coordination, stiff muscles, overactive reflexes, paralysis of one side of the body, blurred vision, double vision, sudden visual loss, temporary loss of vision in one eye, difficulty speaking, slurred speech, speech loss, fatigue, lightheadedness, vertigo, numbness or weakness, pins and needle feeling or reduced sensation of touch, facial muscle weakness or numbness, difficulty swallowing, headache, mental confusion, rapid involuntary eye movement, or combinations thereof.

STING (Stimulator of interferon gene): A signaling molecule associated with the endoplasmic reticulum (ER), essential for controlling the transcription of numerous host defense genes, including type I interferons (IFNs) and pro-inflammatory cytokines, following the recognition of aberrant DNA species or cyclic dinucleotides (CDNs) in the cytosol of the cell. Several type I IFN-inducing compounds, including DMXAA, have been found to function by binding to and triggering STING activity in mice.

STING sequences are publicly available on GenBank. See, for example, Gene Accession number MF622062.1 (human) and MF622063.1 (mouse), each of which are herein incorporated by reference as available on May 4, 2020.

Subject: Living multi-cellular vertebrate organisms, a category that includes human and non-human mammals. The term subject is used interchangeably with patient.

Thrombolytics: Medicines that can be used for emergency treatment of an ischemic stroke (a stroke caused by a blot clot), a heart attack (myocardial infarction), or a massive pulmonary embolism (PE). Thrombolytic drugs can be used to dissolve, or lyse, blood clots (thrombi). Thrombolytic drugs dissolve blood clots by activating plasminogen, which forms a cleaved product called plasmin. Plasmin is a proteolytic enzyme that is capable of breaking cross-links between fibrin molecules, which provide the structural integrity of blood clots. Because of these actions, thrombolytic drugs are also called plasminogen activators and fibrinolytic drugs. There are three major classes of fibrinolytic drugs: tissue plasminogen activator (tPA), streptokinase (SK), and urokinase (UK). While drugs in these three classes all have the ability to effectively dissolve blood clots, they differ in their detailed mechanisms in ways that alter their selectivity for fibrin clots.

Tissue: An aggregate of cells, usually of a particular kind, together with their intercellular substance that form one of the structural materials of an animal and that in animals include connective tissue, epithelium, muscle tissue, and nerve tissue.

TLRs (Toll-like receptors): Receptors that play a critical role in the early innate immune response to invading pathogens by sensing microorganisms and are involved in sensing endogenous danger signals. TLRs are evolutionarily conserved receptors are homologues of the Drosophila Toll protein, discovered to be important for defense against microbial infection. TLRs recognize highly conserved structural motifs known as pathogen-associated microbial patterns (PAMPs), which are exclusively expressed by microbial pathogens, or danger/damage-associated molecular patterns (DAMPs) that are endogenous molecules released from necrotic or dying cells. PAMPs include various bacterial cell wall components such as lipopolysaccharide (LPS), peptidoglycan (PGN) and lipopeptides, as well as flagellin, bacterial DNA and viral double-stranded RNA. DAMPs include intracellular proteins such as heat shock proteins as well as protein fragments from the extracellular matrix. Stimulation of TLRs by the corresponding PAMPs or DAMPs initiates signaling cascades leading to the activation of transcription factors, such as AP-1, NF-κB and interferon regulatory factors (IRFs). Signaling by TLRs result in a variety of cellular responses including the production of interferons (IFNs), pro-inflammatory cytokines and effector cytokines that direct the adaptive immune response.

TLR2 sequences are publicly available on GenBank. See, for example, Gene Accession number NM_001318787.2 (human) and NM_011905.3 (mouse), each of which are herein incorporated by reference as available on May 4, 2020.

TLR3 sequences are publicly available on GenBank. See, for example, Gene Accession number NM_003265.3 (human) and NM_001357316.1 (mouse), each of which are herein incorporated by reference as available on May 4, 2020.

TLR4 sequences are publicly available on GenBank. See, for example, Gene Accession number NM_138554.5 (human) and NM_021297.3 (mouse), each of which are herein incorporated by reference as available on May 4, 2020.

TLR9 sequences are publicly available on GenBank. See, for example, Gene Accession number NM_017442.3 (human) and NM_031178.2 (mouse), each of which are herein incorporated by reference as available on May 4, 2020.

TNF-α (Tumor necrosis factor alpha): A multifunctional cytokine secreted primarily by macrophages, natural killer (NK) cells, and lymphocytes. A primary role of TNF is in the regulation of immune cells. TNF-α as used herein may also be referred to as TNF.

TNF-alpha sequences are publicly available on GenBank. See, for example, Gene Accession number NM_000594.4 (human) and NM_001278601.1 (mouse), each of which are herein incorporated by reference as available on May 4, 2020.

Toxicity index: A ratio of a CC50 (50% cytotoxic concentration) to EC50 (half-maximal effective concentration) for a compound of the present disclosure.

Treating a disease: A therapeutic intervention that ameliorates a sign or symptom of a disease or pathological condition including but not limited to a stroke, such as a sign or symptom of stroke. Treatment can induce remission or cure of a condition or slow progression, for example, in some instances can include inhibiting the full development of a disease, for example preventing development adverse conditions associated with a stroke. Prevention of a disease does not require a total absence of disease. For example, a decrease of at least 20%, such as at least 20%, at least 25%, at least 30%, at least 40%, or at least 50% can be sufficient.

Treating a disease can be a reduction in severity of some or all clinical symptoms of the disease or condition, a reduction in the number of relapses of the disease or condition, an improvement in the overall health or well-being of the subject, by other parameters well known in the art that are specific to the particular disease or condition, and combinations of such factors. It may be understood that treating a disease as discussed is not limited to stroke (e.g., ischemic stroke), but also includes, but is not limited to, cancer, multiple sclerosis, viral infection, and chronic fatigue syndrome as disclosed herein.

TRIF (Toll/IL-1R-domain-containing adapter-inducing interferon-β): An adapter in responding to activation of toll-like receptors (TLRs). TRIF-dependent signaling is involved in TLR-mediated production of type-I IFN and several other inflammatory mediators. Various pathogens target the signaling molecules and transcriptional regulators acting in the TRIF pathway. The TRIF pathway contributes to control of both viral and bacterial pathogens through promotion of inflammatory mediators and activation of antimicrobial responses. TRIF-dependent signaling has both protective and pathologic roles in several chronic inflammatory disease conditions, as well as in wound-repair processes.

TRIF sequences are publicly available on GenBank. See, for example, Gene Accession number NM_182919.3 (human) and NM_174989.5 (mouse), each of which are herein incorporated by reference as available on May 4, 2020.

TRIM30 (tripartite motif protein 30): A member of the tripartite motif (TRIM) protein family involved in the regulation of cell proliferation, differentiation, development, oncogenesis, apoptosis and antiviral responses. The TRIM protein family is an expanding family of RING (‘really interesting new gene’) proteins, also known as RBCC proteins as they contain an RBCC motif, which comprises a RING domain, one or two B-boxes and a predicted coiled-coil region.

TRIM30 sequences are publicly available on GenBank. See, for example, Gene Accession number BC005447.1 (mouse), herein incorporated by reference as available on May 4, 2020.

Under conditions sufficient for: A phrase that is used to describe any environment that permits the desired activity. One example includes administering a disclosed agent to a subject under conditions sufficient to allow the desired activity. In particular examples, the desired activity is increasing the expression or activity of IRF3 (and/or other IRFs, including but not limited to IRF7).

USP18 (ubiquitin specific peptidase 18): Belongs to the ubiquitin-specific family of enzymes that cleave ubiquitin from ubiquitinated protein substrates. Known to efficiently cleave ISG15 (a ubiquitin-like protein) fusions, and deletion of this gene in mice has been shown to result in a massive increase of ISG15 conjugates in tissues, indicating that this protein is a ISG15-specific protease.

USP18 sequences are publicly available on GenBank. See, for example, Gene Accession number NM_017414.4 (human) and NM_011909.2 (mouse), each of which are herein incorporated by reference as available on May 4, 2020.

Wild-type: A strain, gene or characteristic which prevails among individuals in natural conditions, as distinct from an atypical mutant type.

ZBP1 (Z-DNA binding protein 1): Plays a role in the innate immune response by binding to DNA and inducing type-I interferon production. Participates in the detection by the host's innate immune system of DNA from viral, bacterial or even host origin. Plays a role in host defense against tumors and pathogens. Acts as a cytoplasmic DNA sensor which, when activated, induces the recruitment of TBK1 and IRF3 to its C-terminal region and activates the downstream interferon regulatory factor (IRF) and NF-kappa B transcription factors, leading to type-I interferon production.

### III. Compounds for Altering Expression and/or Activity of IRFs

Disclosed herein are compounds that may be used as modulatory agents for interferon regulatory factors (IRFs) in methods disclosed herein. In particular disclosed embodiments, one or more of the disclosed compounds is effective in treating stroke. The compound is a small-molecule therapeutic. In particular disclosed embodiments, the small-molecule therapeutic is a cyclic compound comprising a heteroatom-containing skeleton. In certain embodiments, the cyclic compound has a general formula (A1-L-B1) illustrated below at Table 1, where A1 may comprise but is not limited to a functional group as depicted by the various functional groups at Group A of Table 1, where B1 is comprised at least of a functional group as depicted at Group B of Table 1, and where L may be understood to comprise a linkage group. It may be understood that the functional groups depicted by group A and group B may include analogs/derivatives thereof, without departing from the scope of this disclosure.

TABLE 1
High-level formula comprising ND and NB
chemofamilies of the present disclosure

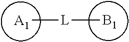

| Group A | Group B |
|---|---|
| 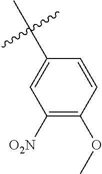 [SMILES: *c1ccc(OC)c([N+](=O)[O-])c1] | 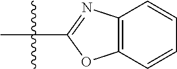 [SMILES: *c1nc2ccccc2o1] |
| 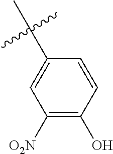 [SMILES: *c1ccc(O)c([N+](=O)[O-])c1] | |
| 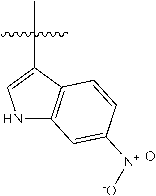 | |
| 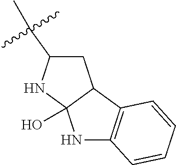 [SMILES: *C1CC2c3ccccc3NC2(O)N1] | |

As a more specific example of that described above at Table 1, in certain embodiments the cyclic compound has a general formula exemplified by that depicted at Table 2. At Table 2, example functional groups comprising each of R1, R2, R3, R4 and R5 are as depicted therein. Furthermore, ring A and B are illustrated, and the region in parenthesis may comprise a reversible amide bond where the bond to the amide nitrogen can be either to atom 1 of ring A or atom 1 of ring B. It may be understood that the functional groups depicted as R1-R5 at Table 2 may not be specifically limited those portrayed, but may include analogs/derivatives thereof, without departing from the scope of this disclosure. For example, R4 may more generally comprise an electronegative group, and R1 may more generally comprise a hydrophobic group. In some examples, R3 and R5 may more generally comprise a substituent larger than a proton. In some examples where a halogen is included at R4, a methyl group at R5 may be preferable to a methoxy group. In some examples where a nitro group is included at R4, a methoxy group at R5 may be preferable to other substitutions (e.g., methyl). Rings A and B at Table 2 may be understood to be of 4-7 atoms in any combination of carbon, nitrogen, oxygen and sulfur constituents.

TABLE 2
Mid-level formula for compounds of the ND and NB Chemofamilies

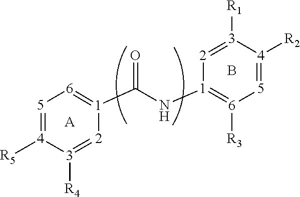

| R₁ | R₂ | R₃ | R₄ | R₅ |
|---|---|---|---|---|
| 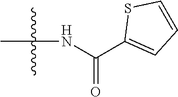 [SMILES: *NC(=O)c1cccs1] | 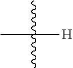 [SMILES: *[H]] | 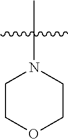 [SMILES: *N1CCOCC1] | halogen | 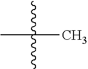 |
| 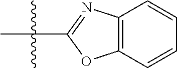 [SMILES: *c1nc2ccccc2o1] | 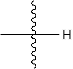 [SMILES: *[H]] | 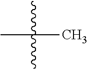 | 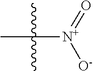 [SMILES: *[N+](=O)[O-]] | 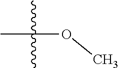 [SMILES: *OC] |

Taking into account the disclosure above at Table 1 and Table 2, in some embodiments the cyclic compound is of the general formula as that depicted at Table 3 (top), where a more specific formula 1 (middle) relates to NB series analogs as discussed herein, and the more specific formula 2 (bottom) relates to the ND series analogs discussed herein.

With regard to Table 3, the region in parenthesis may comprise but is not limited to a reversible amide bond, similar to that discussed above with regard to Table 2 (e.g., amide nitrogen can be either to atom 1 of ring A or atom 1 of ring B). In some examples, a different linker may be used without departing from the scope of this disclosure. For R1 at Table 3, m may be zero, 1, 2, 3, 4, or 5. For R2 at Table 3, m may be zero, 1, 2, 3, or 4. For R3 at Table 3, m may be zero, 1 2, 3 or 4. When R1 is greater than 1, it may be understood that R1 at different substituent positions may comprise different functional groups, or the same functional group, or some mixture thereof. When R2 is greater than 1, it may be understood that R2 at different substituent positions may comprise different functional groups, or the same functional group, or some mixture thereof. When R3 is greater than 1, it may be understood that R3 at different substituent positions may comprise different functional groups, or the same functional group, or some mixture thereof. Rings A and B at Table 3 may be understood to be of 4-7 atoms in any combination of carbon, nitrogen, oxygen and sulfur constituents. X at Table 3 may be understood to be one of carbon, nitrogen, oxygen and phosphorous constituents. Various exemplary R groups (e.g., R1, R2, R3) for specific formula 1 and specific formula 2 at Table 3 are depicted by compounds listed in Table 8 below, but it may be understood that the present disclosure encompasses other R groups not specifically depicted by the compounds listed in Table 8 including but not limited to analogs/derivatives of the R groups of the compounds listed in Table 8.

TABLE 3
Chemical formulas comprising the ND and NB chemofamilies
General Formula

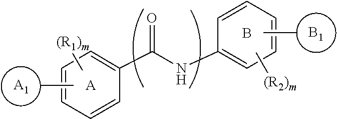

Specific Formula 1 (NB series analogs)

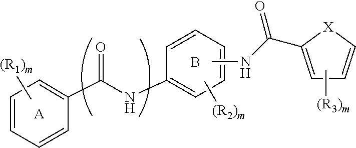

Specific Formula 2 (ND series analogs)

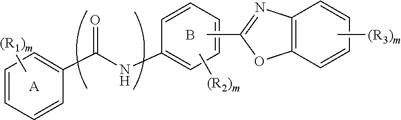

In other embodiments as discussed herein, the cyclic compound has a general formula (A1-L-B1) illustrated below at Table 4, similar to that discussed above at Table 1, but where A1 is comprised at least of a functional group as depicted by the various functional groups at Group A at Table 4, where B1 is comprised at least of a functional group depicted by the functional groups depicted at Group B of Table 4, and where L may be understood to comprise a linkage group. It may be understood that the functional groups depicted by Group A and Group B at Table 4 may include analogs/derivatives thereof, without departing from the scope of this disclosure.

TABLE 4
High-level formula comprising NA and NC chemofamilies
of the present disclosure

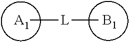

| Group A | Group B |
|---|---|
| 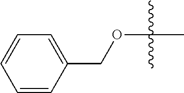 [SMILES: *OCc1ccccc1] | 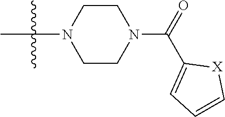 |
| 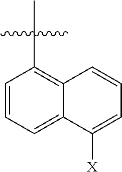 | 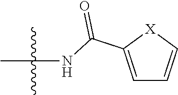 |
| 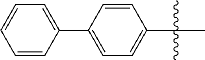 [SMILES: *c1ccc(-c2ccccc2)cc1] | |
| 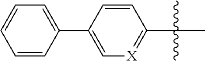 | |

As a more specific example of that described above at Table 4, in certain embodiments the cyclic compound has a general formula exemplified by that depicted at Table 5, which may be understood to be the same general formula exemplified by that depicted at Table 2 above, but where the R groups (R1-5) of Table 5 are different than those depicted at Table 2. However, it may be understood that R groups depicted at Table 2 may not be excluded from inclusion with regard to Table 5, and vice versa. As an example, an R5 group depicted at Table 2 may be included as an R5 group at Table 5, or vice versa, without departing from the scope of this disclosure.

TABLE 5
Mid-level formula for compounds of the NA and NC Chemofamilies

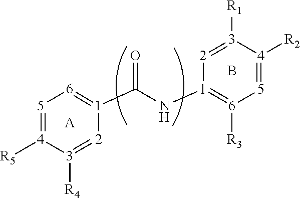

| R₁ | R₂ | R₃ | R₄ | R₅ |
|---|---|---|---|---|
| 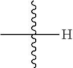 [SMILES: *[H]] | 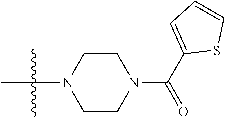 [SMILES: *N1CCN(C(=O)c2cccs2)CC1] | 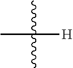 [SMILES: *[H]] | 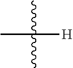 [SMILES: *[H]] | 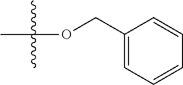 [SMILES: *OCc1ccccc1] |
| 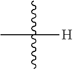 [SMILES: *[H]] | 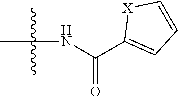 | 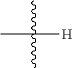 [SMILES: *[H]] | 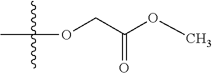 [SMILES: *OCC(=O)OC] | 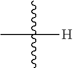 [SMILES: *[H]] |
| | | | 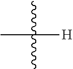 [SMILES: *[H]] | 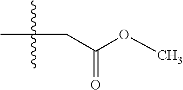 [SMILES: *CC(=O)OC] |

As shown at Table 5, groups R1-R5 may be selected from the groups shown, in any combination. Ring A and B are indicated at Table 5, and the region in parenthesis may be understood to comprise a reversible amide bond, similar to that discussed above, but other linker regions are encompassed by the present disclosure. At Table 5, the region in parenthesis may comprise a reversible amide bond in some examples, where the bond to the amide nitrogen can be either to atom 1 of ring A or atom 1 of ring B. It may be understood that the functional groups depicted as R1-R5 (and even rings A and B) may not be specifically limited to those portrayed, but may include analogs/derivatives thereof, without departing from the scope of this disclosure. For example, additional modifications to the benzyloxy group at R5 of Table 5 are contemplated. As another example, ring A at Table 5 may be replaced by a halogenated naphthalene. A halogenated naphthalene may produce a human active compound, but may be inactive in a murine background. A nitrogen at position 2 or 3 of ring A may similarly show species specific effects, and both may be active in a human background. For example, a nitrogen at position 2 of ring A may result in a loss of activity in a mouse background. A nitrogen at position 3 of ring A may restore such a loss in activity. In molecules with a biphenyl moiety, a heteroatom at position 2 may be better for eliciting both human and mouse activity. Rings A and B at Table 5 may be understood to be of 4-7 atoms in any combination of carbon, nitrogen, oxygen and sulfur constituents.

Taking into account the disclosure above at Table 4 and Table 5, in some embodiments the cyclic compound is of the general formula as that depicted at Table 6 (top), where a more specific formula 1 relates to NC series analogs as discussed herein, and the more specific formula 2 relates to the NA series analogs discussed herein.

TABLE 6
Chemical formulas comprising the NA and NC chemofamilies
General Formula

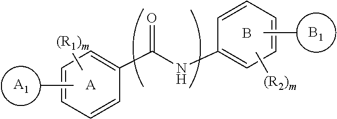

Specific Formula 1 (NC series analogs)

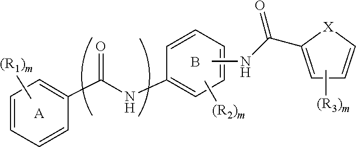

Specific Formula 2 (NA series analogs)

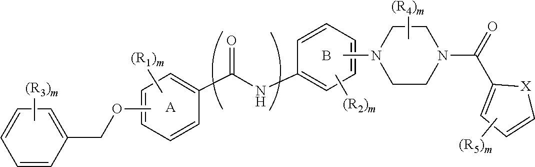

With regard to Table 6, the region in parenthesis may comprise a reversible amide bond, similar to that discussed above with regard to Table 5 (e.g., amide nitrogen can be either to atom 1 of ring A or atom 1 of ring B). In some examples, a different linker may be used without departing from the scope of this disclosure. For R1 at Table 6, m may be zero, 1, 2, 3, 4 or 5. For R2 at Table 6, m may be zero, 1, 2, 3, or 4. For R3 at Table 6, m may be zero, 1 2, 3 or 4. For R4 at Table 6, m may be zero, 1, 2, 3, or 4. For R5 at Table 6, m may be zero 1, 2, or 3. When R1 is greater than 1, it may be understood that R1 at different substituent positions may comprise different functional groups, or the same functional group, or some mixture thereof. When R2 is greater than 1, it may be understood that R2 at different substituent positions may comprise different functional groups, or the same functional group, or some mixture thereof. When R3 is greater than 1, it may be understood that R3 at different substituent positions may comprise different functional groups, or the same functional group, or some mixture thereof. When R4 is greater than 1, it may be understood that R4 at different substituent positions may comprise different functional groups, or the same functional group, or some mixture thereof. When R5 is greater than 1, it may be understood that R5 at different substituent positions may comprise different functional groups, or the same functional group, or some mixture thereof. Rings A and B at Table 6 may be understood to be of 4-7 atoms in any combination of carbon, nitrogen, oxygen and sulfur constituents. X at Table 6 may be understood to be one of carbon, nitrogen, oxygen and phosphorous constituents. Various exemplary R groups (e.g., R1, R2, R3, R4, R5) for specific formula 1 and specific formula 2 with regard to Table 6 are illustrated by compounds listed in Table 7 below, but it may be understood that other R groups are encompassed by the present disclosure, including but not limited to analogs/derivatives of the R groups depicted by compounds listed in Table 7 below.

Exemplary compounds of the present disclosure, many of which are encompassed by the specific formula 1 or the specific formula 2 above at Table 3, or the specific formula 1 or the specific formula 2 at Table 6, are provided in Tables 7-8, respectively, below:

TABLE 7
Exemplary compounds of the NA and NC Chemofamilies

| Structure Picture/Molecular Weight | Molecular Weight | Common Name |
|---|---|---|
| 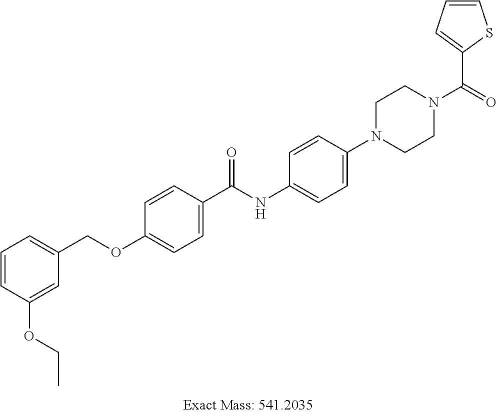 [SMILES: CCOc1cccc(COc2ccc(C(=O)Nc3ccc(N4CCN(C(=O)c5cccs5)CC4)cc3)cc2)c1] | 541 | NA42 |
| 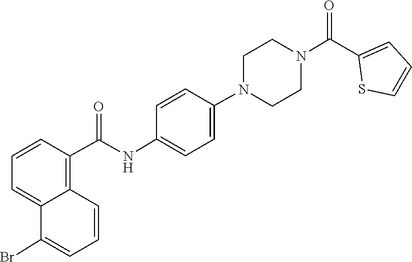 [SMILES: O=C(Nc1ccc(N2CCN(C(=O)c3cccs3)CC2)cc1)c1cccc2c(Br)cccc12] | 520.44 | NA-21 |
| 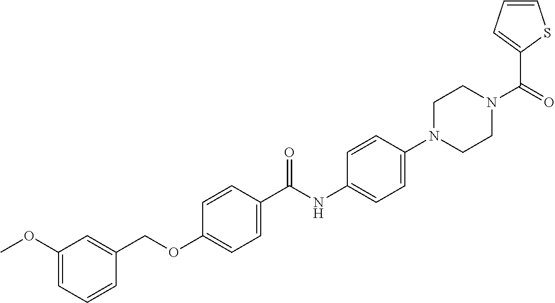 [SMILES: COc1cccc(COc2ccc(C(=O)Nc3ccc(N4CCN(C(=O)c5cccs5)CC4)cc3)cc2)c1] | 527 | NA-24 |
| 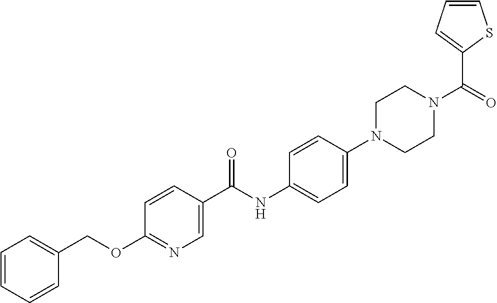 [SMILES: O=C(Nc1ccc(N2CCN(C(=O)c3cccs3)CC2)cc1)c1ccc(OCc2ccccc2)nc1] | 498 | NA-59 |
| 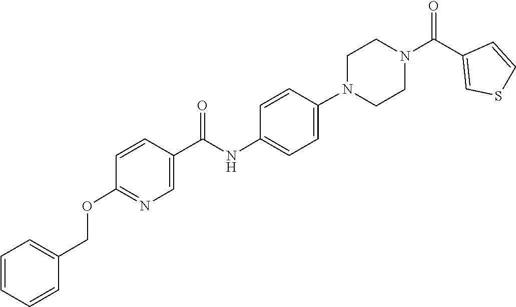 [SMILES: O=C(Nc1ccc(N2CCN(C(=O)c3ccsc3)CC2)cc1)c1ccc(OCc2ccccc2)nc1] | 498 | NA-1 |
| 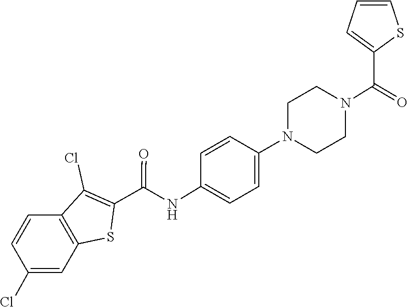 [SMILES: O=C(Nc1ccc(N2CCN(C(=O)c3cccs3)CC2)cc1)c1sc2cc(Cl)ccc2c1Cl] | 515 | NA-20 |
| 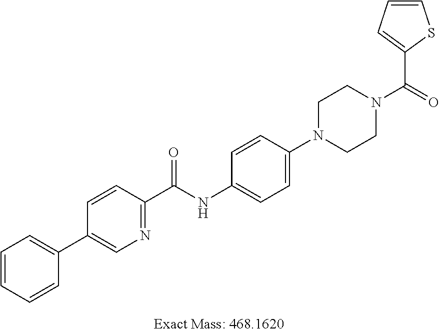 [SMILES: O=C(Nc1ccc(N2CCN(C(=O)c3cccs3)CC2)cc1)c1ccc(-c2ccccc2)cn1] | 468 | NA-39 |
| 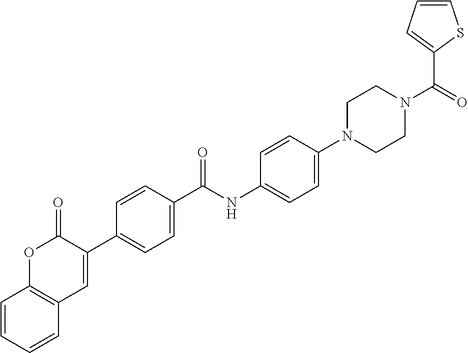 [SMILES: O=C(Nc1ccc(N2CCN(C(=O)c3cccs3)CC2)cc1)c1ccc(-c2cc3ccccc3oc2=O)cc1] | 535 | NA-32 |
| 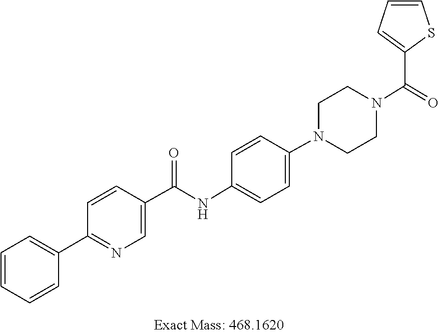 [SMILES: O=C(Nc1ccc(N2CCN(C(=O)c3cccs3)CC2)cc1)c1ccc(-c2ccccc2)nc1] | 468 | NA-41 |
| 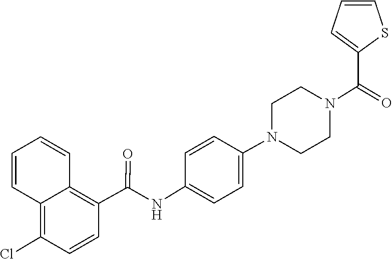 [SMILES: O=C(Nc1ccc(N2CCN(C(=O)c3cccs3)CC2)cc1)c1ccc(Cl)c2ccccc12] | 476 | NA13 |
| 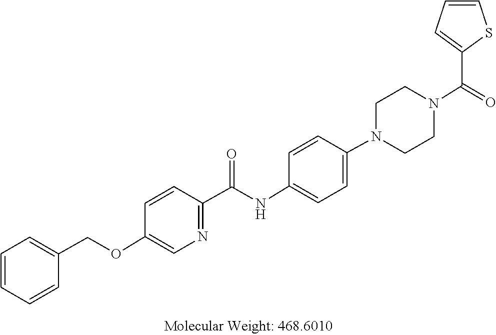 [SMILES: O=C(Nc1ccc(N2CCN(C(=O)c3cccs3)CC2)cc1)c1ccc(OCc2ccccc2)cn1] | 498 | NA-60 |
| 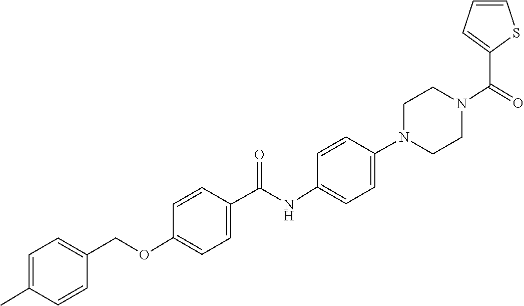 [SMILES: Cc1ccc(COc2ccc(C(=O)Nc3ccc(N4CCN(C(=O)c5cccs5)CC4)cc3)cc2)cc1] | 519 | NA-25 |
| 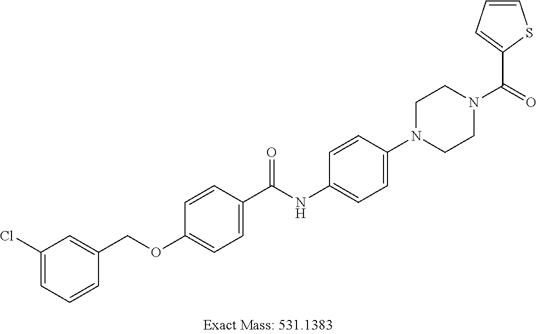 [SMILES: O=C(Nc1ccc(N2CCN(C(=O)c3cccs3)CC2)cc1)c1ccc(OCc2cccc(Cl)c2)cc1] | 531 | NA-33 |
| 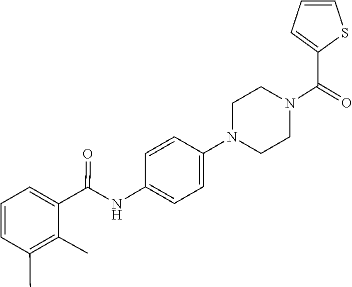 [SMILES: Cc1cccc(C(=O)Nc2ccc(N3CCN(C(=O)c4cccs4)CC3)cc2)c1C] | 419 | NA-6 |
| 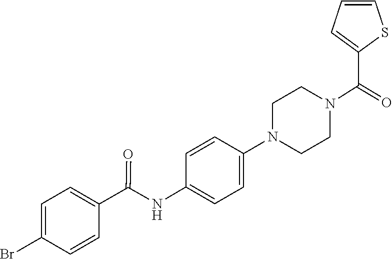 [SMILES: O=C(Nc1ccc(N2CCN(C(=O)c3cccs3)CC2)cc1)c1ccc(Br)cc1] | 469 | NA-14 |
| 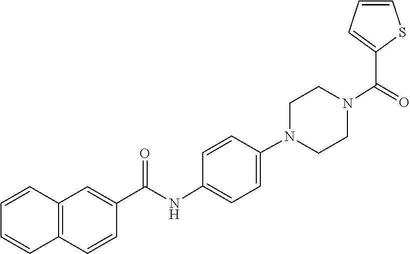 [SMILES: O=C(Nc1ccc(N2CCN(C(=O)c3cccs3)CC2)cc1)c1ccc2ccccc2c1] | 441 | NA-7 |
| 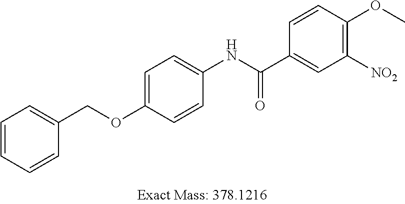 [SMILES: COc1ccc(C(=O)Nc2ccc(OCc3ccccc3)cc2)cc1[N+](=O)[O-]] | 378 | NA-44 |
| 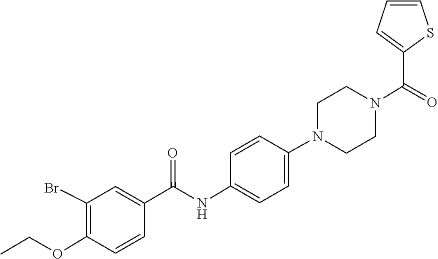 [SMILES: CCOc1ccc(C(=O)Nc2ccc(N3CCN(C(=O)c4cccs4)CC3)cc2)cc1Br] | 513 | NA-30 |
| 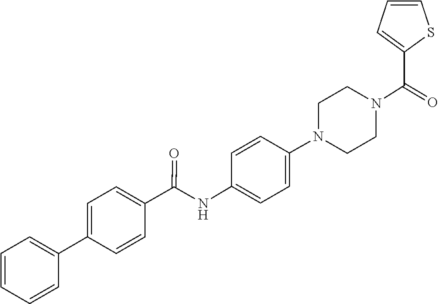 [SMILES: O=C(Nc1ccc(N2CCN(C(=O)c3cccs3)CC2)cc1)c1ccc(-c2ccccc2)cc1] | 467 | NA-19 |
| 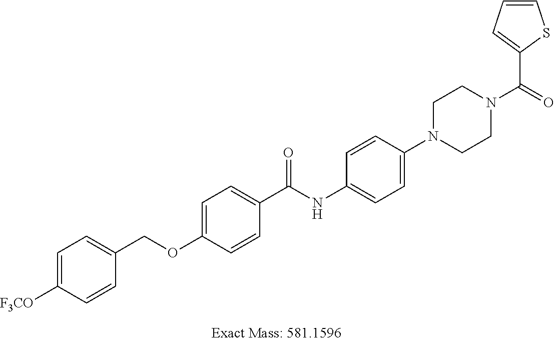 [SMILES: O=C(Nc1ccc(N2CCN(C(=O)c3cccs3)CC2)cc1)c1ccc(OCc2ccc(OC(F)(F)F)cc2)cc1] | 581 | NA-34 |
| 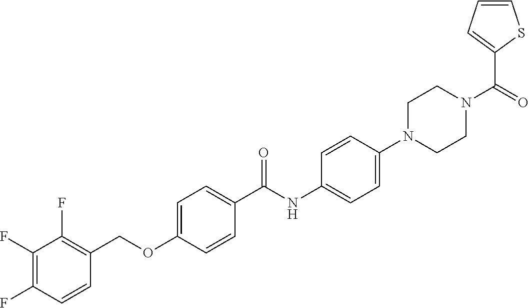 [SMILES: O=C(Nc1ccc(N2CCN(C(=O)c3cccs3)CC2)cc1)c1ccc(OCc2ccc(F)c(F)c2F)cc1] | 551 | NA-23 |
| 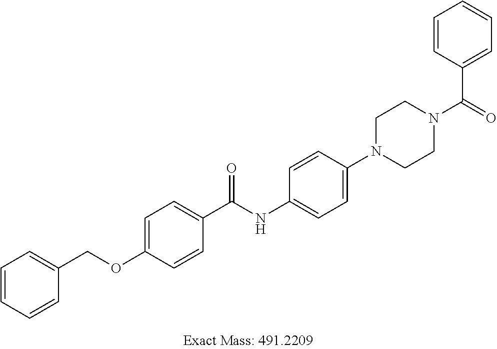 [SMILES: O=C(Nc1ccc(N2CCN(C(=O)c3ccccc3)CC2)cc1)c1ccc(OCc2ccccc2)cc1] | 491 | NA-51 |
| 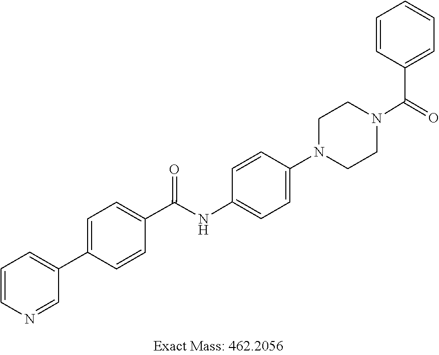 [SMILES: O=C(Nc1ccc(N2CCN(C(=O)c3ccccc3)CC2)cc1)c1ccc(-c2cccnc2)cc1] | 462 | NA-53 |
| 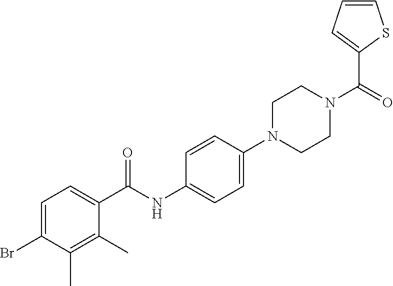 [SMILES: Cc1c(Br)ccc(C(=O)Nc2ccc(N3CCN(C(=O)c4cccs4)CC3)cc2)c1C] | 497 | NA-16 |
| 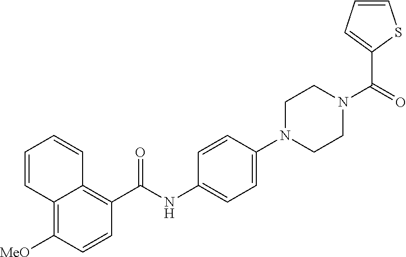 [SMILES: COc1ccc(C(=O)Nc2ccc(N3CCN(C(=O)c4cccs4)CC3)cc2)c2ccccc12] | 471 | NA-15 |
| 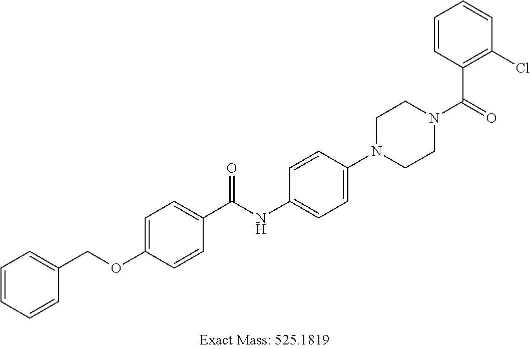 [SMILES: O=C(Nc1ccc(N2CCN(C(=O)c3ccccc3Cl)CC2)cc1)c1ccc(OCc2ccccc2)cc1] | 505 | NA-50 |
| 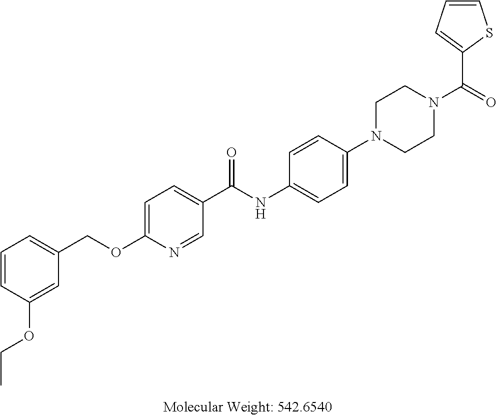 [SMILES: CCOc1cccc(COc2ccc(C(=O)Nc3ccc(N4CCN(C(=O)c5cccs5)CC4)cc3)cn2)c1] | 542 | NA-71 |
| 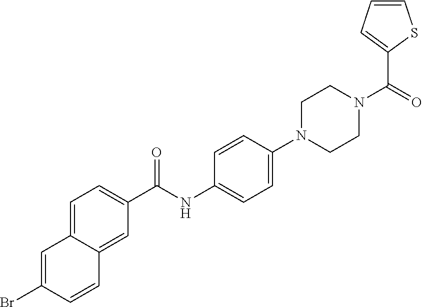 [SMILES: O=C(Nc1ccc(N2CCN(C(=O)c3cccs3)CC2)cc1)c1ccc2cc(Br)ccc2c1] | 519 | NA-17 |
| 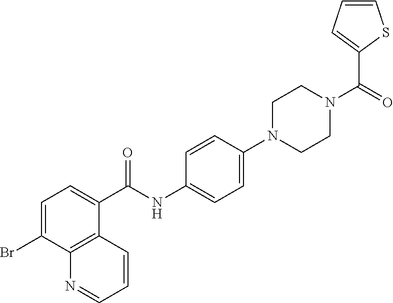 [SMILES: O=C(Nc1ccc(N2CCN(C(=O)c3cccs3)CC2)cc1)c1ccc(Br)c2ncccc12] | 520 | NA-18 |
| 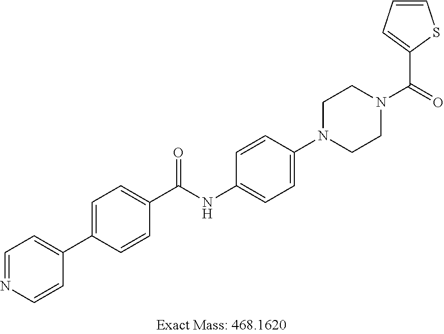 [SMILES: O=C(Nc1ccc(N2CCN(C(=O)c3cccs3)CC2)cc1)c1ccc(-c2ccncc2)cc1] | 468 | NA-38 |
| 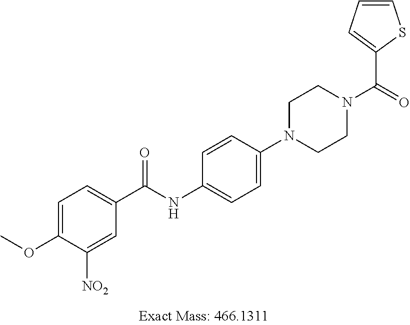 [SMILES: COc1ccc(C(=O)Nc2ccc(N3CCN(C(=O)c4cccs4)CC3)cc2)cc1[N+](=O)[O-]] | 466 | NA-46 |
| 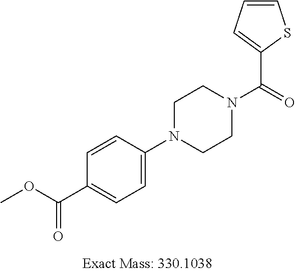 [SMILES: COC(=O)c1ccc(N2CCN(C(=O)c3cccs3)CC2)cc1] | 330 | NA-47 |
| 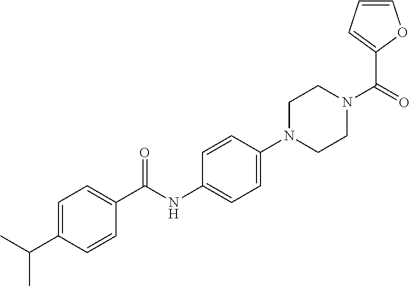 [SMILES: CC(C)c1ccc(C(=O)Nc2ccc(N3CCN(C(=O)c4ccco4)CC3)cc2)cc1] | 417.5 | NA-4 |
| 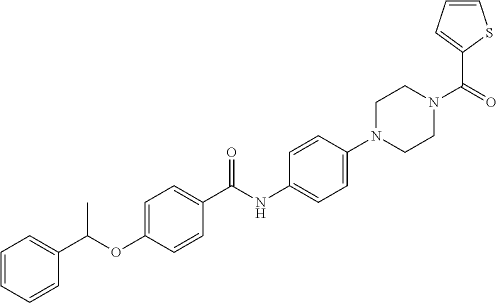 [SMILES: CC(Oc1ccc(C(=O)Nc2ccc(N3CCN(C(=O)c4cccs4)CC3)cc2)cc1)c1ccccc1] | 511 | NA-22 |
| 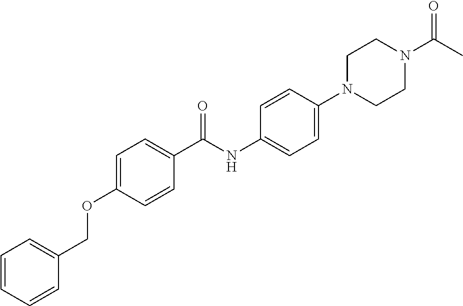 [SMILES: CC(=O)N1CCN(c2ccc(NC(=O)c3ccc(OCc4ccccc4)cc3)cc2)CC1] | 429.5 | NA-2 |
| 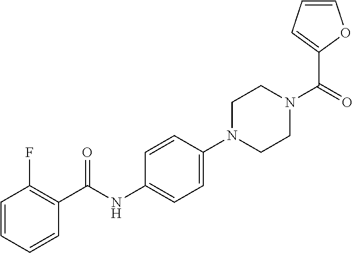 [SMILES: O=C(Nc1ccc(N2CCN(C(=O)c3ccco3)CC2)cc1)c1ccccc1F] | 393.4 | NA-3 |
| 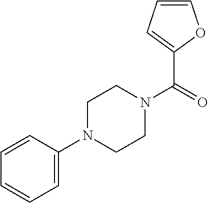 [SMILES: O=C(c1ccco1)N1CCN(c2ccccc2)CC1] | 256.3 | NA-5 |
| 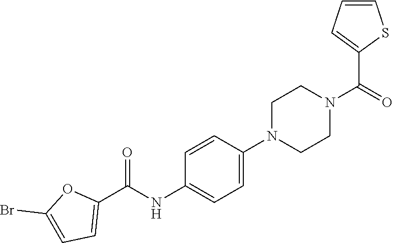 [SMILES: O=C(Nc1ccc(N2CCN(C(=O)c3cccs3)CC2)cc1)c1ccc(Br)o1] | 459 | NA-8 |
| 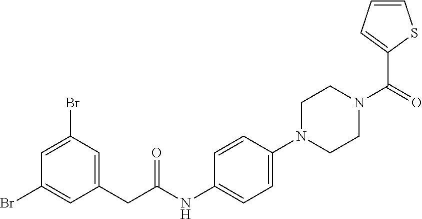 [SMILES: O=C(Cc1cc(Br)cc(Br)c1)Nc1ccc(N2CCN(C(=O)c3cccs3)CC2)cc1] | 560 | NA-9 |
| 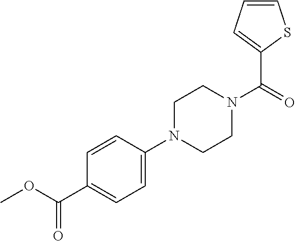 [SMILES: COC(=O)c1ccc(N2CCN(C(=O)c3cccs3)CC2)cc1] | 330 | NA-10 |
| 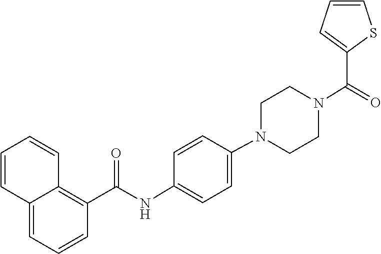 [SMILES: O=C(Nc1ccc(N2CCN(C(=O)c3cccs3)CC2)cc1)c1cccc2ccccc12] | 441 | NA-11 |
| 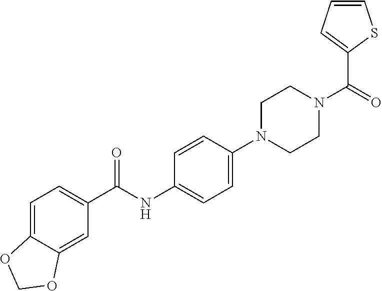 [SMILES: O=C(Nc1ccc(N2CCN(C(=O)c3cccs3)CC2)cc1)c1ccc2c(c1)OCO2] | 435 | NA-12 |
| 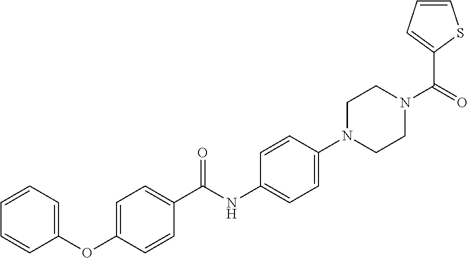 [SMILES: O=C(Nc1ccc(N2CCN(C(=O)c3cccs3)CC2)cc1)c1ccc(Oc2ccccc2)cc1] | 483 | NA-26 |
| 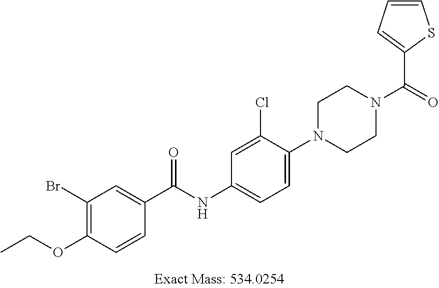 [SMILES: CCOc1ccc(C(=O)Nc2ccc(N3CCN(C(=O)c4cccs4)CC3)c(Cl)c2)cc1Br] | 534 | NA-27 |
| 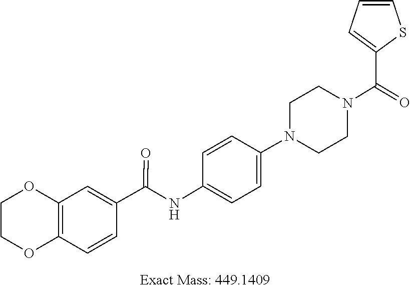 [SMILES: O=C(Nc1ccc(N2CCN(C(=O)c3cccs3)CC2)cc1)c1ccc2c(c1)OCCO2] | 449 | NA-28 |
| 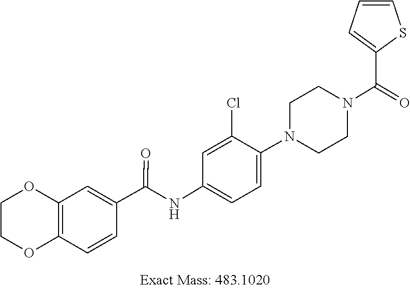 [SMILES: O=C(Nc1ccc(N2CCN(C(=O)c3cccs3)CC2)c(Cl)c1)c1ccc2c(c1)OCCO2] | 483 | NA-29 |
| 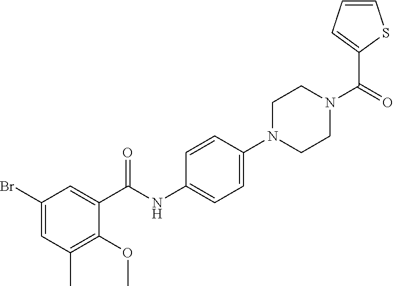 [SMILES: COc1c(C)cc(Br)cc1C(=O)Nc1ccc(N2CCN(C(=O)c3cccs3)CC2)cc1] | 513 | NA-31 |
| 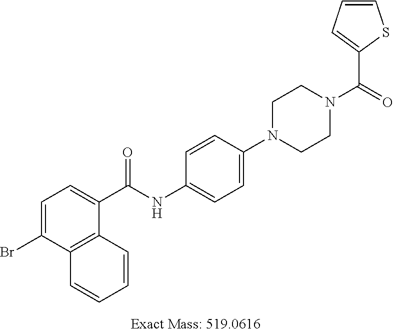 [SMILES: O=C(Nc1ccc(N2CCN(C(=O)c3cccs3)CC2)cc1)c1ccc(Br)c2ccccc12] | 519 | NA-35 |
| 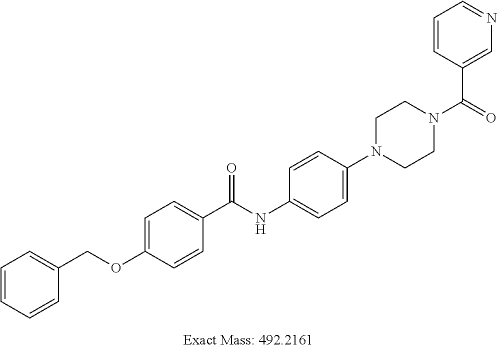 [SMILES: O=C(Nc1ccc(N2CCN(C(=O)c3cccnc3)CC2)cc1)c1ccc(OCc2ccccc2)cc1] | 492 | NA-36 |
| 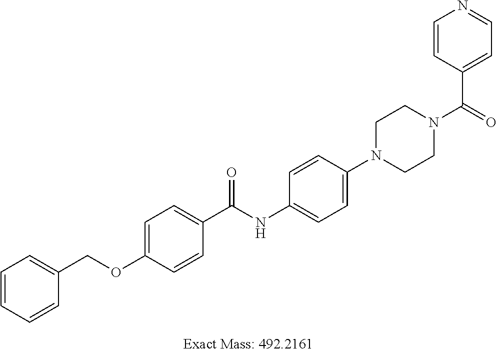 [SMILES: O=C(Nc1ccc(N2CCN(C(=O)c3ccncc3)CC2)cc1)c1ccc(OCc2ccccc2)cc1] | 492 | NA-37 |
| 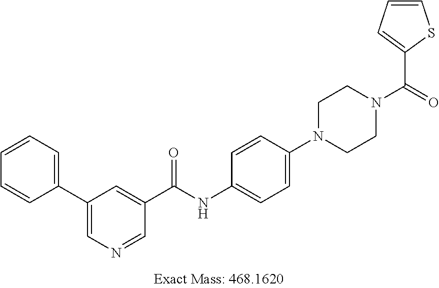 [SMILES: O=C(Nc1ccc(N2CCN(C(=O)c3cccs3)CC2)cc1)c1cncc(-c2ccccc2)c1] | 468 | NA-40 |
| 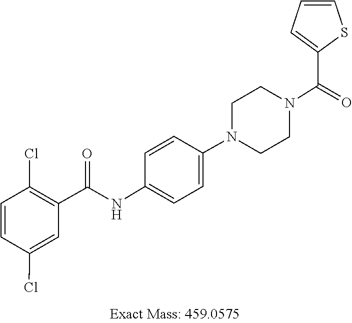 [SMILES: O=C(Nc1ccc(N2CCN(C(=O)c3cccs3)CC2)cc1)c1cc(Cl)ccc1Cl] | 459 | NA-43 |
| 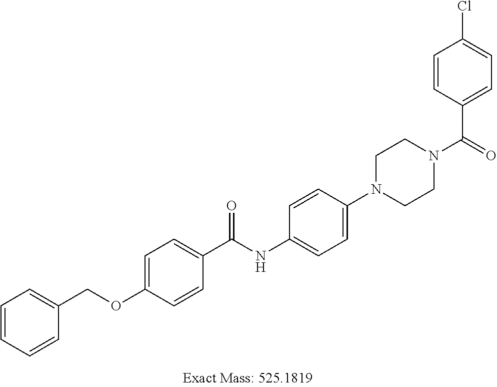 [SMILES: O=C(Nc1ccc(N2CCN(C(=O)c3ccc(Cl)cc3)CC2)cc1)c1ccc(OCc2ccccc2)cc1] | 525 | NA-45 |
| 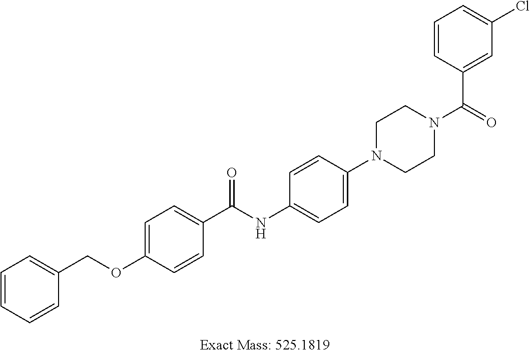 [SMILES: O=C(Nc1ccc(N2CCN(C(=O)c3cccc(Cl)c3)CC2)cc1)c1ccc(OCc2ccccc2)cc1] | 525 | NA-48 |
| 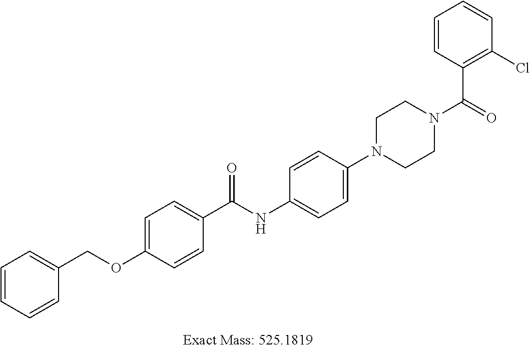 [SMILES: O=C(Nc1ccc(N2CCN(C(=O)c3ccccc3Cl)CC2)cc1)c1ccc(OCc2ccccc2)cc1] | 525 | NA-49 |
| 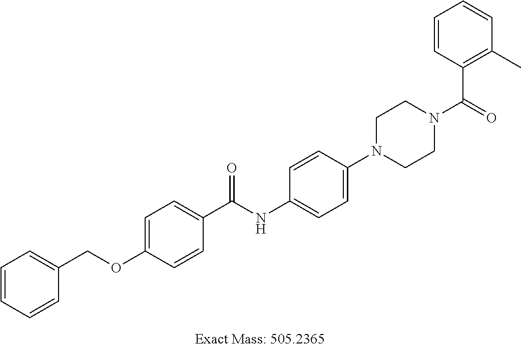 [SMILES: Cc1ccccc1C(=O)N1CCN(c2ccc(NC(=O)c3ccc(OCc4ccccc4)cc3)cc2)CC1] | 505 | NA-52 |
| 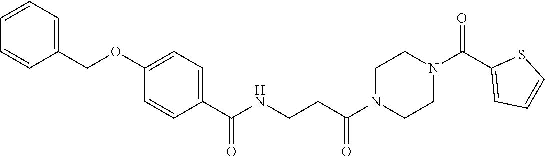 [SMILES: O=C(NCCC(=O)N1CCN(C(=O)c2cccs2)CC1)c1ccc(OCc2ccccc2)cc1] | 477.58 | NA-54 |
| 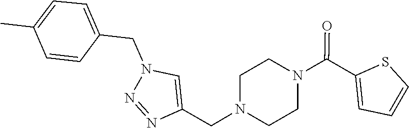 [SMILES: Cc1ccc(Cn2cc(CN3CCN(C(=O)c4cccs4)CC3)nn2)cc1] | 381.5 | NA-55 |
| 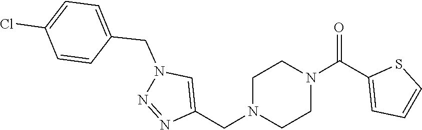 [SMILES: O=C(c1cccs1)N1CCN(Cc2cn(Cc3ccc(Cl)cc3)nn2)CC1] | 401.91 | NA-56 |
| 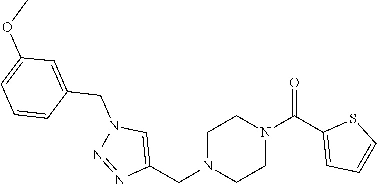 [SMILES: COc1cccc(Cn2cc(CN3CCN(C(=O)c4cccs4)CC3)nn2)c1] | 397.5 | NA-57 |
| 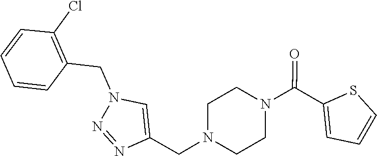 [SMILES: O=C(c1cccs1)N1CCN(Cc2cn(Cc3ccccc3Cl)nn2)CC1] | 401.91 | NA-58 |
| 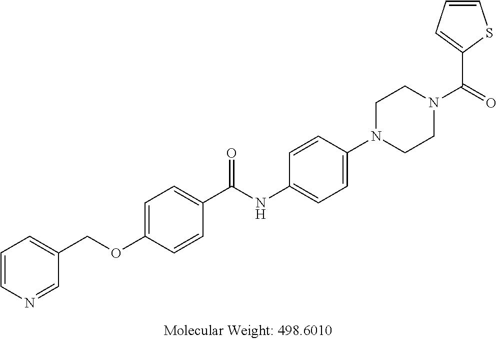 [SMILES: O=C(Nc1ccc(N2CCN(C(=O)c3cccs3)CC2)cc1)c1ccc(OCc2cccnc2)cc1] | 498 | NA-61 |
| 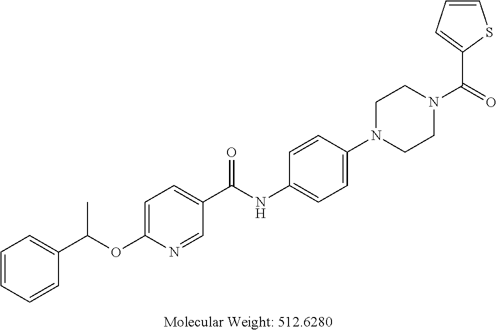 [SMILES: CC(Oc1ccc(C(=O)Nc2ccc(N3CCN(C(=O)c4cccs4)CC3)cc2)cn1)c1ccccc1] | 512 | NA-62 |
| 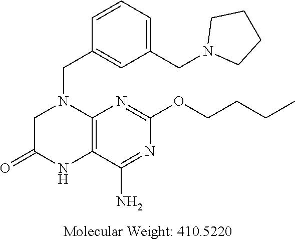 [SMILES: CCCCOc1nc(N)c2c(n1)N(Cc1cccc(CN3CCCC3)c1)CC(=O)N2] | 410 | NA-63 |
| 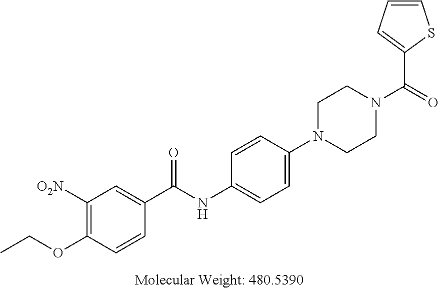 [SMILES: CCOc1ccc(C(=O)Nc2ccc(N3CCN(C(=O)c4cccs4)CC3)cc2)cc1[N+](=O)[O-]] | 480 | NA-65 |
| 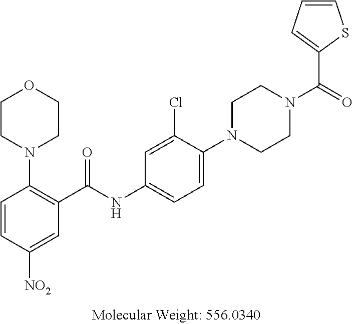 [SMILES: O=C(Nc1ccc(N2CCN(C(=O)c3cccs3)CC2)c(Cl)c1)c1cc([N+](=O)[O-])ccc1N1CCOCC1] | 556 | NA-66 |
| 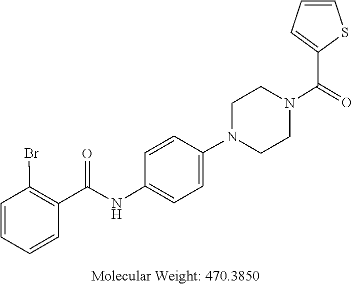 [SMILES: O=C(Nc1ccc(N2CCN(C(=O)c3cccs3)CC2)cc1)c1ccccc1Br] | 470 | NA-67 |
| 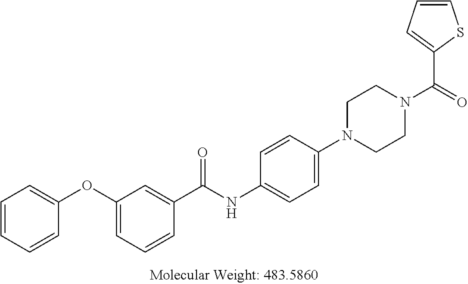 [SMILES: O=C(Nc1ccc(N2CCN(C(=O)c3cccs3)CC2)cc1)c1cccc(Oc2ccccc2)c1] | 483 | NA-68 |
| 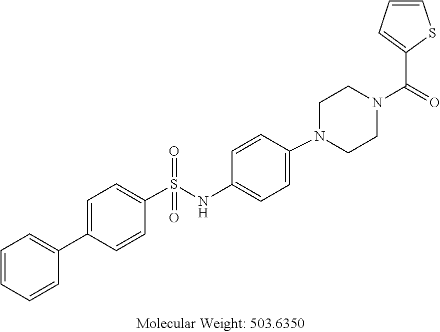 [SMILES: O=C(c1cccs1)N1CCN(c2ccc(NS(=O)(=O)c3ccc(-c4ccccc4)cc3)cc2)CC1] | 503 | NA-69 |
| 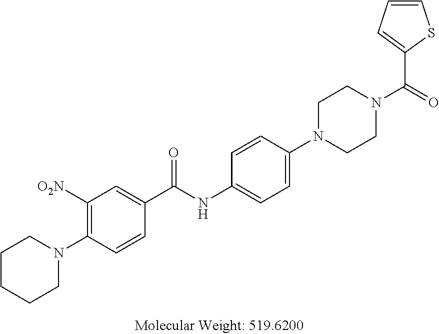 [SMILES: O=C(Nc1ccc(N2CCN(C(=O)c3cccs3)CC2)cc1)c1ccc(N2CCCCC2)c([N+](=O)[O-])c1] | 520 | NA-70 |
| 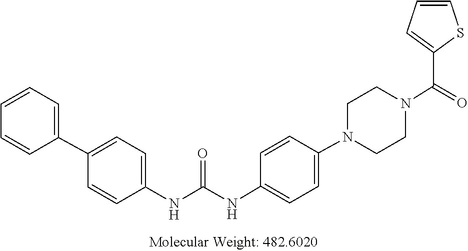 [SMILES: O=C(Nc1ccc(-c2ccccc2)cc1)Nc1ccc(N2CCN(C(=O)c3cccs3)CC2)cc1] | 482 | NA-72 |
| 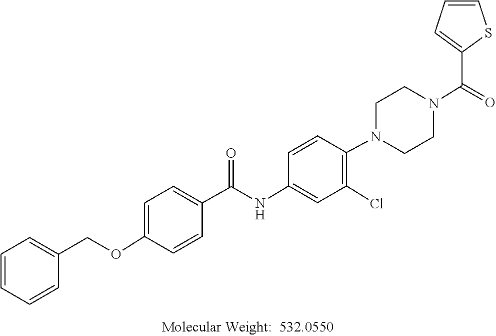 [SMILES: O=C(Nc1ccc(N2CCN(C(=O)c3cccs3)CC2)c(Cl)c1)c1ccc(OCc2ccccc2)cc1] | 532 | NA-73 |
| 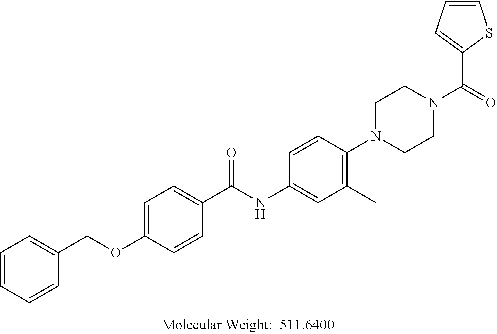 [SMILES: Cc1cc(NC(=O)c2ccc(OCc3ccccc3)cc2)ccc1N1CCN(C(=O)c2cccs2)CC1] | 511 | NA-74 |
| 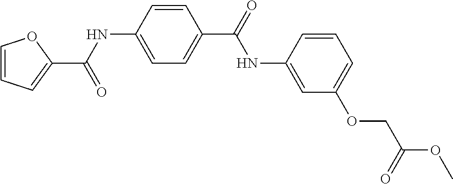 [SMILES: COC(=O)COc1cccc(NC(=O)c2ccc(NC(=O)c3ccco3)cc2)c1] | 394.4 | NC-1 |
| 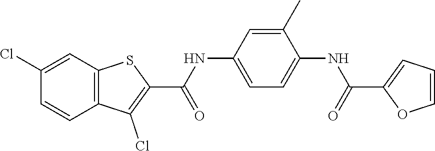 [SMILES: Cc1cc(NC(=O)c2sc3cc(Cl)ccc3c2Cl)ccc1NC(=O)c1ccco1] | 445.3 | NC-2 |
| 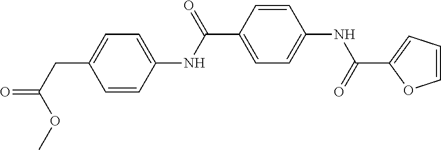 [SMILES: COC(=O)Cc1ccc(NC(=O)c2ccc(NC(=O)c3ccco3)cc2)cc1] | 378.4 | NC-3 |
| 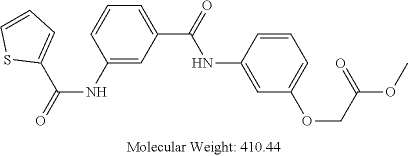 [SMILES: COC(=O)COc1cccc(NC(=O)c2cccc(NC(=O)c3cccs3)c2)c1] | 410.44 | NC-4 |

TABLE 8
Exemplary compounds of the ND and NB Chemofamilies

| Structure Picture/Molecular Weight | Molecular Weight | Common Name |
|---|---|---|
| 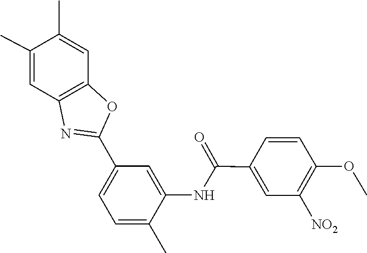 | 431 | ND13 |
| 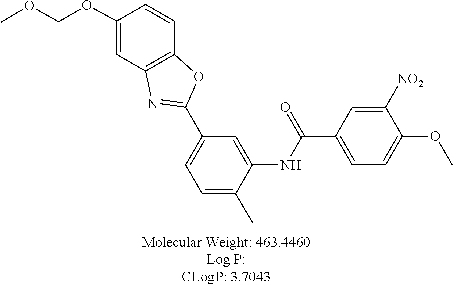 | 464 | ND-95 |
| 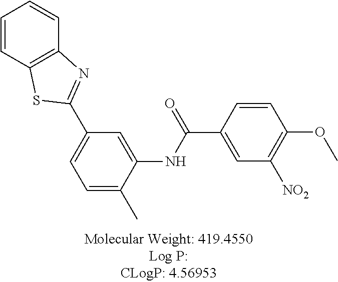 [SMILES: COc1ccc(C(=O)Nc2cc(-c3nc4ccccc4s3)ccc2C)cc1[N+](=O)[O-]] | 419 | ND52 |
| 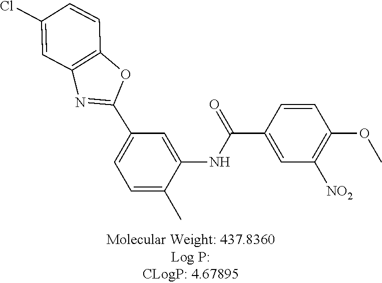 [SMILES: COc1ccc(C(=O)Nc2cc(-c3nc4cc(Cl)ccc4o3)ccc2C)cc1[N+](=O)[O-]] | 438 | ND-40 |
| 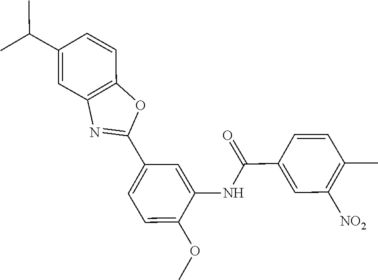 [SMILES: COc1ccc(-c2nc3cc(C(C)C)ccc3o2)cc1NC(=O)c1ccc(C)c([N+](=O)[O-])c1] | 445 | ND-16 |
| 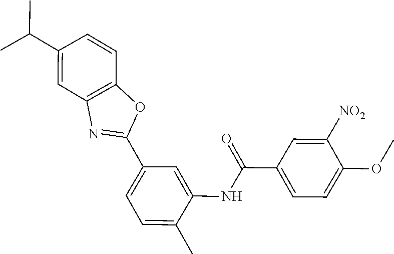 [SMILES: COc1ccc(C(=O)Nc2cc(-c3nc4cc(C(C)C)ccc4o3)ccc2C)cc1[N+](=O)[O-]] | 445.47 | ND-2 |
| 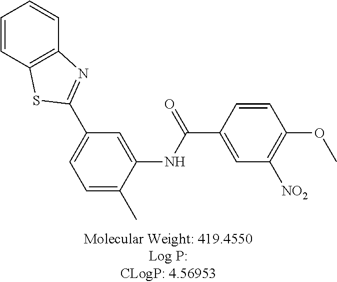 [SMILES: COc1ccc(C(=O)Nc2cc(-c3nc4ccccc4s3)ccc2C)cc1[N+](=O)[O-]] | 419 | ND-38 |
| 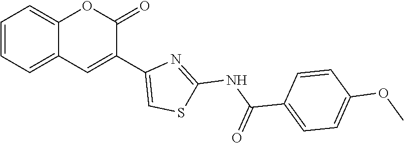 [SMILES: COc1ccc(C(=O)Nc2nc(-c3cc4ccccc4oc3=O)cs2)cc1] | 378.4 | ND-1 |
| 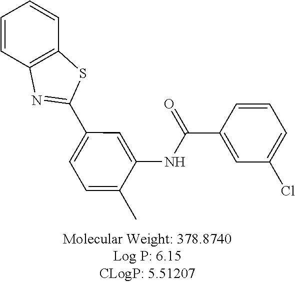 [SMILES: Cc1ccc(-c2nc3ccccc3s2)cc1NC(=O)c1cccc(Cl)c1] | 378 | ND-80 |
| 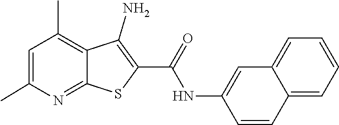 [SMILES: Cc1cc(C)c2c(N)c(C(=O)Nc3ccc4ccccc4c3)sc2n1] | 347.43 | ND-6 |
| 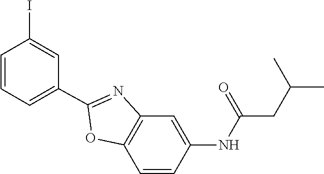 [SMILES: CC(C)CC(=O)Nc1ccc2oc(-c3cccc(I)c3)nc2c1] | 420.24 | ND-5 |
| 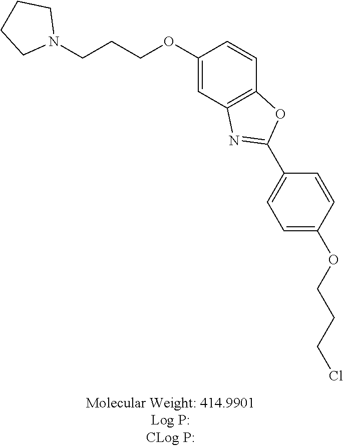 [SMILES: ClCCCOc1ccc(-c2nc3cc(OCCCN4CCCC4)ccc3o2)cc1] | 414 | ND-94 |
| 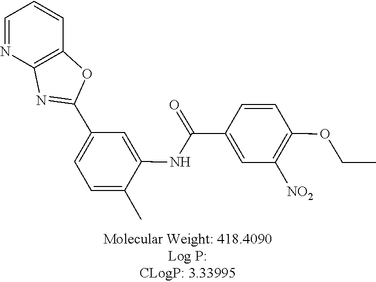 [SMILES: CCOc1ccc(C(=O)Nc2cc(-c3nc4ncccc4o3)ccc2C)cc1[N+](=O)[O-]] | 418 | ND-48 |
| 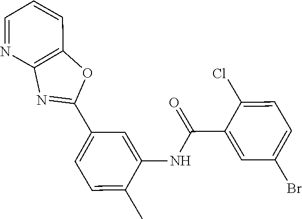 [SMILES: Cc1ccc(-c2nc3ncccc3o2)cc1NC(=O)c1cc(Br)ccc1Cl] | 442.5 | ND-3 |
| 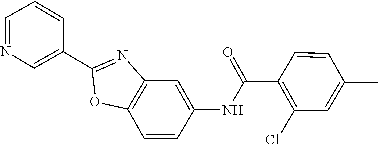 [SMILES: Cc1ccc(C(=O)Nc2ccc3oc(-c4cccnc4)nc3c2)c(Cl)c1] | 364 | ND-4 |
| 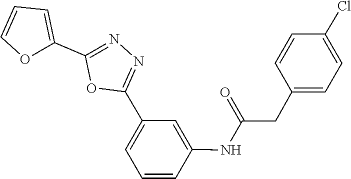 [SMILES: O=C(Cc1ccc(Cl)cc1)Nc1cccc(-c2nnc(-c3ccco3)o2)c1] | 380 | ND-7 |
| 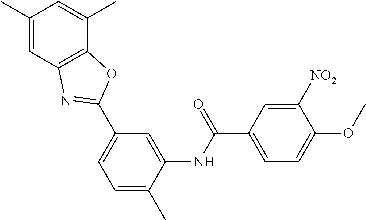 | 431 | ND-8 |
| 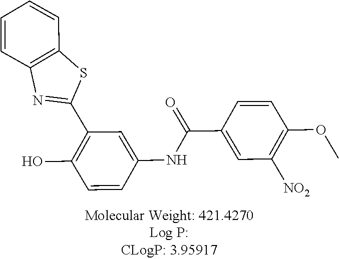 [SMILES: COc1ccc(C(=O)Nc2ccc(O)c(-c3nc4ccccc4s3)c2)cc1[N+](=O)[O-]] | 421 | ND-65 |
| 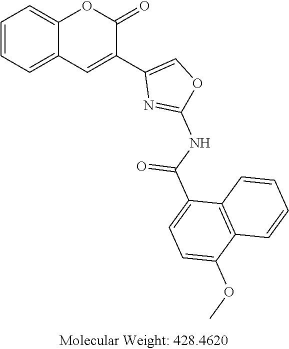 [SMILES: COc1ccc(C(=O)Nc2nc(-c3cc4ccccc4oc3=O)co2)c2ccccc12] | 428 | ND-18 |
| 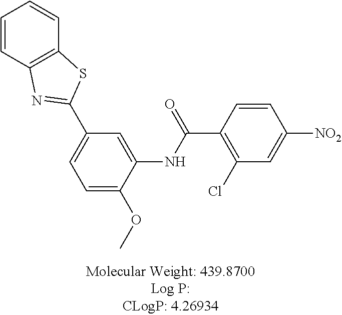 [SMILES: COc1ccc(-c2nc3ccccc3s2)cc1NC(=O)c1ccc([N+](=O)[O-])cc1Cl] | 439 | ND-67 |
| 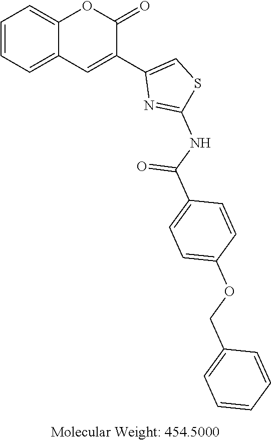 [SMILES: O=C(Nc1nc(-c2cc3ccccc3oc2=O)cs1)c1ccc(OCc2ccccc2)cc1] | 454 | ND-22 |
| 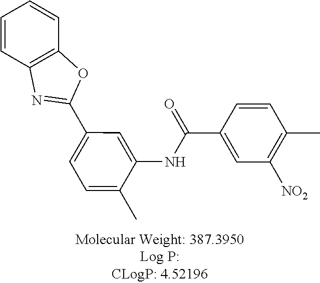 [SMILES: Cc1ccc(-c2nc3ccccc3o2)cc1NC(=O)c1ccc(C)c([N+](=O)[O-])c1] | 387 | ND-30 |
| 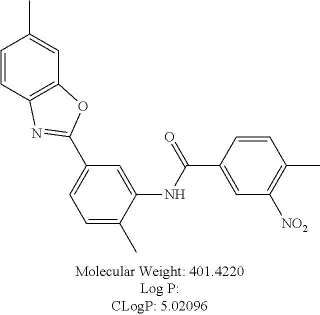 [SMILES: Cc1ccc2nc(-c3ccc(C)c(NC(=O)c4ccc(C)c([N+](=O)[O-])c4)c3)oc2c1] | 401 | ND-31 |
| 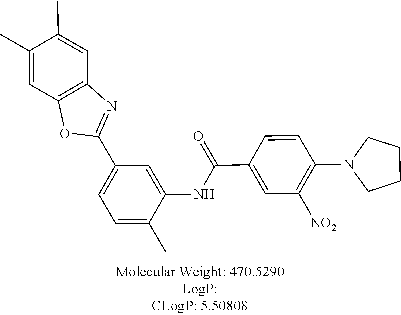 [SMILES: Cc1cc2nc(-c3ccc(C)c(NC(=O)c4ccc(N5CCCC5)c([N+](=O)[O-])c4)c3)oc2cc1C] | 470 | ND-34 |
| 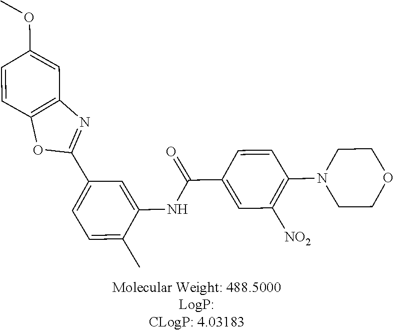 [SMILES: COc1ccc2oc(-c3ccc(C)c(NC(=O)c4ccc(N5CCOCC5)c([N+](=O)[O-])c4)c3)nc2c1] | 488 | ND-35 |
| 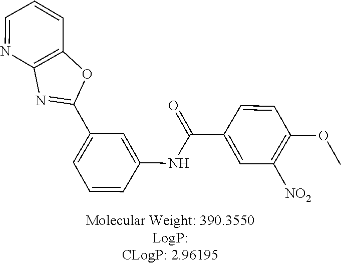 [SMILES: COc1ccc(C(=O)Nc2cccc(-c3nc4ncccc4o3)c2)cc1[N+](=O)[O-]] | 390 | ND-41 |
| 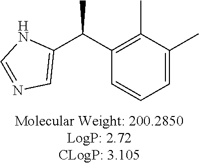 | 200 | ND-76 |
| 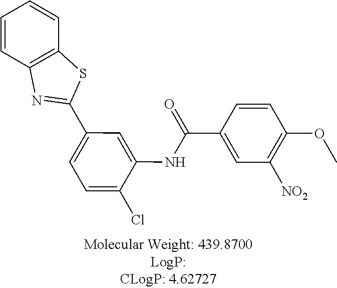 [SMILES: COc1ccc(C(=O)Nc2cc(-c3nc4ccccc4s3)ccc2Cl)cc1[N+](=O)[O-]] | 474 | ND-89 |
| 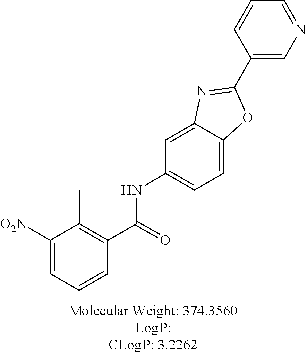 [SMILES: Cc1c(C(=O)Nc2ccc3oc(-c4cccnc4)nc3c2)cccc1[N+](=O)[O-]] | 374 | ND-42 |
| 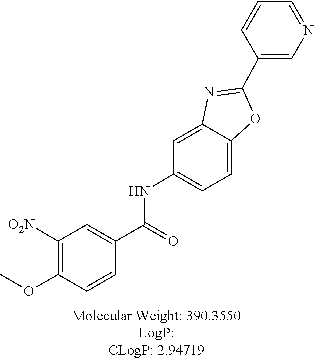 [SMILES: COc1ccc(C(=O)Nc2ccc3oc(-c4cccnc4)nc3c2)cc1[N+](=O)[O-]] | 390 | ND-43 |
| 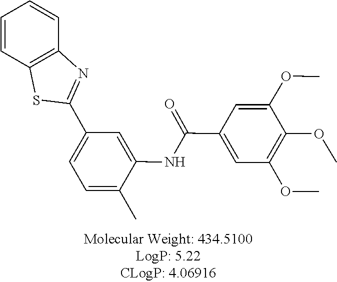 [SMILES: COc1cc(C(=O)Nc2cc(-c3nc4ccccc4s3)ccc2C)cc(OC)c1OC] | 418 | ND-37 |
| 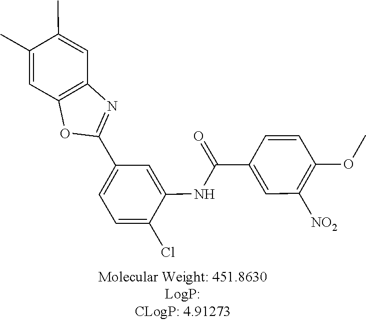 | 452 | ND-50 |
| 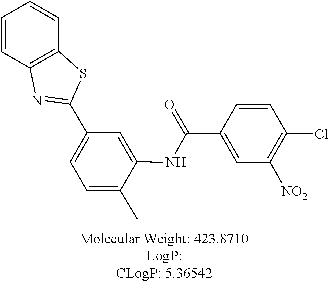 [SMILES: Cc1ccc(-c2nc3ccccc3s2)cc1NC(=O)c1ccc(Cl)c([N+](=O)[O-])c1] | 424 | ND-64 |
| 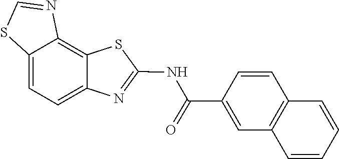 [SMILES: O=C(Nc1nc2ccc3scnc3c2s1)c1ccc2ccccc2c1] | 361 | ND-58 |
| 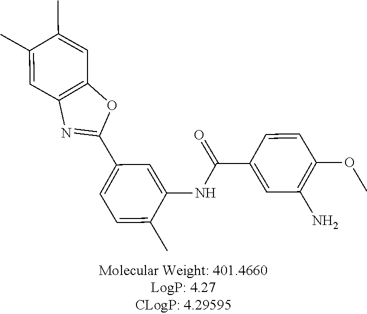 | 401 | ND-60 |
| 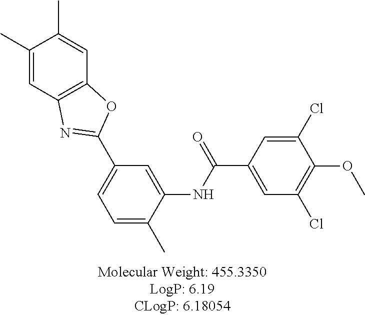 | 455 | ND-69 |
| 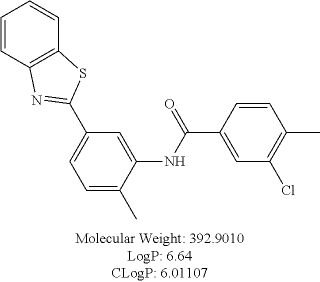 [SMILES: Cc1ccc(C(=O)Nc2cc(-c3nc4ccccc4s3)ccc2C)cc1Cl] | 393 | ND-70 |
| 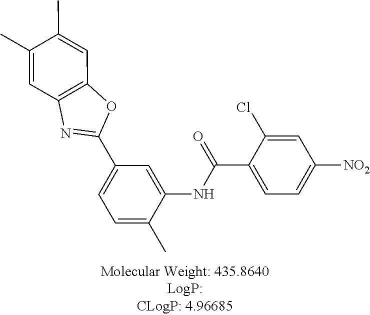 [SMILES: Cc1cc2nc(-c3ccc(C)c(NC(=O)c4ccc([N+](=O)[O-])cc4Cl)c3)oc2cc1C] | 435 | ND-102 |
| 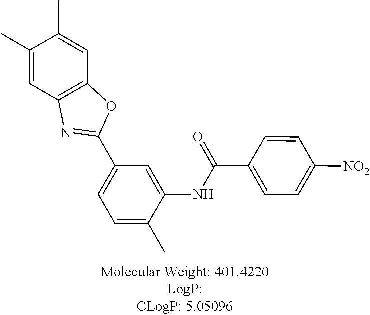 [SMILES: Cc1cc2nc(-c3ccc(C)c(NC(=O)c4ccc([N+](=O)[O-])cc4)c3)oc2cc1C] | 401 | ND-71 |
| 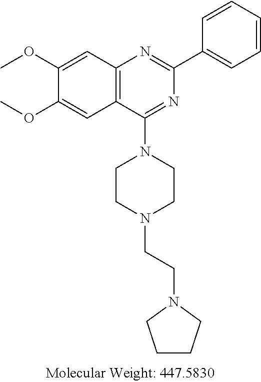 [SMILES: COc1cc2nc(-c3ccccc3)nc(N3CCN(CCN4CCCC4)CC3)c2cc1OC] | 447 | ND-98 |
| 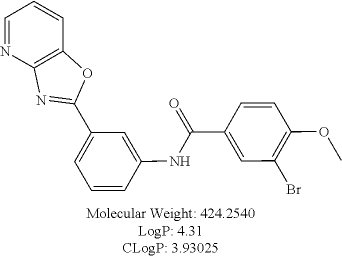 [SMILES: COc1ccc(C(=O)Nc2cccc(-c3nc4ncccc4o3)c2)cc1Br] | 438 | ND-47 |
| 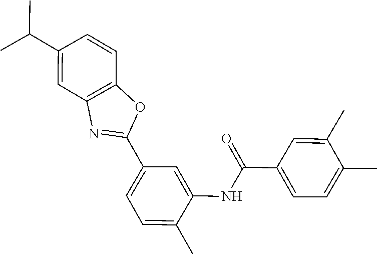 [SMILES: Cc1ccc(C(=O)Nc2cc(-c3nc4cc(C(C)C)ccc4o3)ccc2C)cc1C] | 398 | ND-9 |
| 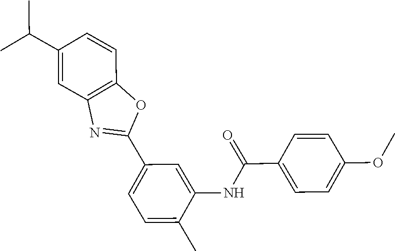 [SMILES: COc1ccc(C(=O)Nc2cc(-c3nc4cc(C(C)C)ccc4o3)ccc2C)cc1] | 400 | ND-10 |
| 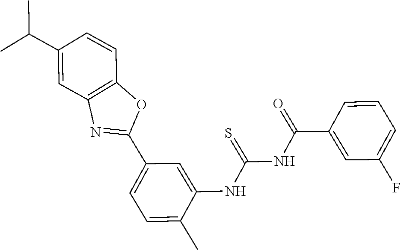 [SMILES: Cc1ccc(-c2nc3cc(C(C)C)ccc3o2)cc1NC(=S)NC(=O)c1cccc(F)c1] | 447 | ND-11 |
| 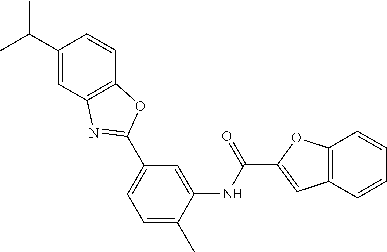 [SMILES: Cc1ccc(-c2nc3cc(C(C)C)ccc3o2)cc1NC(=O)c1cc2ccccc2o1] | 410 | ND-12 |
| 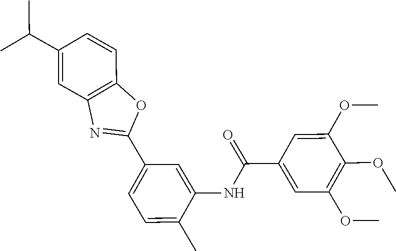 [SMILES: COc1cc(C(=O)Nc2cc(-c3nc4cc(C(C)C)ccc4o3)ccc2C)cc(OC)c1OC] | 461 | ND-14 |
| 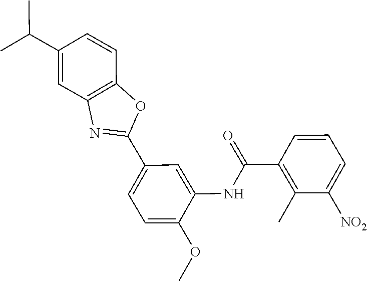 [SMILES: COc1ccc(-c2nc3cc(C(C)C)ccc3o2)cc1NC(=O)c1cccc([N+](=O)[O-])c1C] | 445 | ND-15 |
| 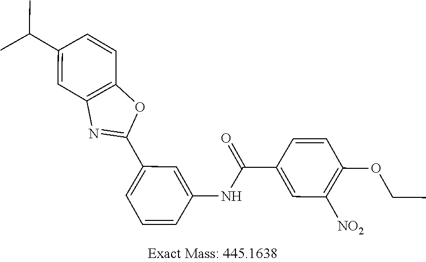 [SMILES: CCOc1ccc(C(=O)Nc2cccc(-c3nc4cc(C(C)C)ccc4o3)c2)cc1[N+](=O)[O-]] | 445 | ND-17 |
| 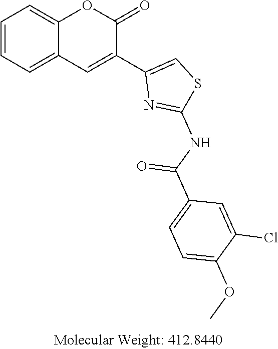 [SMILES: COc1ccc(C(=O)Nc2nc(-c3cc4ccccc4oc3=O)cs2)cc1Cl] | 412 | ND-19 |
| 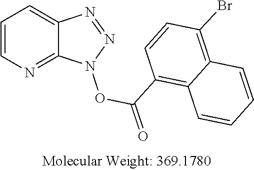 [SMILES: O=C(On1nnc2cccnc21)c1ccc(Br)c2ccccc12] | 369 | ND-20 |
| 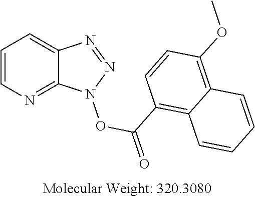 [SMILES: COc1ccc(C(=O)On2nnc3cccnc32)c2ccccc12] | 320 | ND-21 |
| 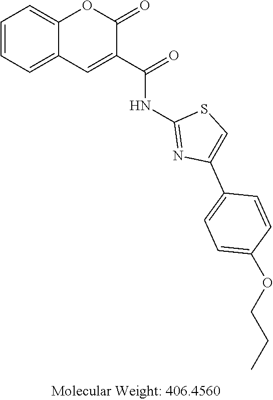 [SMILES: CCCOc1ccc(-c2csc(NC(=O)c3cc4ccccc4oc3=O)n2)cc1] | 406 | ND-23 |
| 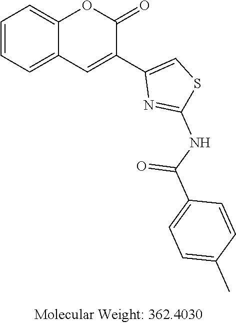 [SMILES: Cc1ccc(C(=O)Nc2nc(-c3cc4ccccc4oc3=O)cs2)cc1] | 362 | ND-24 |
| 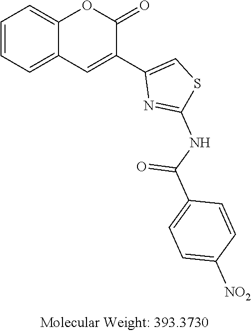 [SMILES: O=C(Nc1nc(-c2cc3ccccc3oc2=O)cs1)c1ccc([N+](=O)[O-])cc1] | 393 | ND-25 |
| 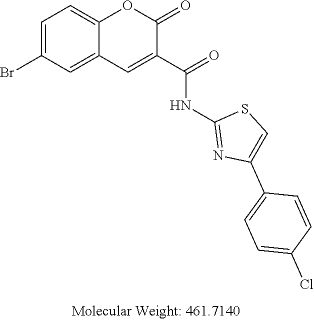 [SMILES: O=C(Nc1nc(-c2ccc(Cl)cc2)cs1)c1cc2cc(Br)ccc2oc1=O] | 462 | ND-26 |
| 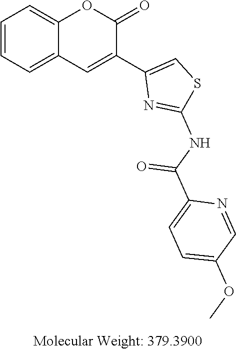 [SMILES: COc1ccc(C(=O)Nc2nc(-c3cc4ccccc4oc3=O)cs2)nc1] | 379 | ND-27 |
| 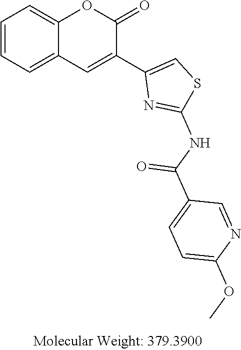 [SMILES: COc1ccc(C(=O)Nc2nc(-c3cc4ccccc4oc3=O)cs2)cn1] | 379 | ND-28 |
| 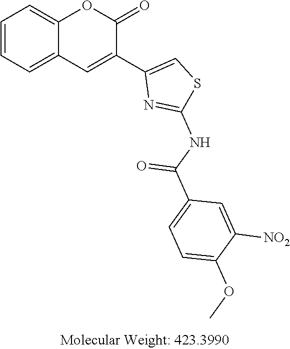 [SMILES: COc1ccc(C(=O)Nc2nc(-c3cc4ccccc4oc3=O)cs2)cc1[N+](=O)[O-]] | 423 | ND-29 |
| 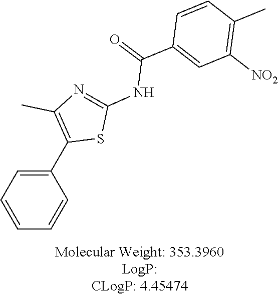 [SMILES: Cc1ccc(C(=O)Nc2nc(C)c(-c3ccccc3)s2)cc1[N+](=O)[O-]] | 353 | ND-32 |
| 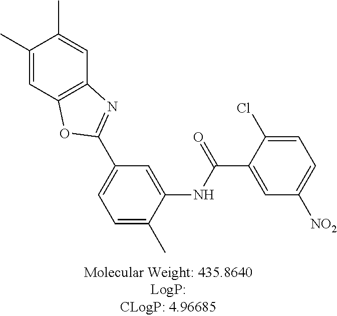 [SMILES: Cc1cc2nc(-c3ccc(C)c(NC(=O)c4cc([N+](=O)[O-])ccc4Cl)c3)oc2cc1C] | 435 | ND-33 |
| 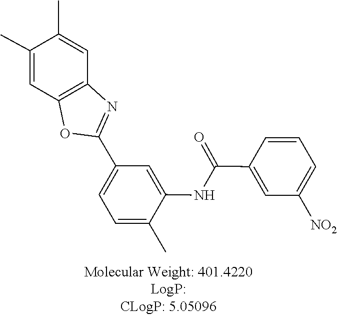 [SMILES: Cc1cc2nc(-c3ccc(C)c(NC(=O)c4cccc([N+](=O)[O-])c4)c3)oc2cc1C] | 401 | ND-36 |
| 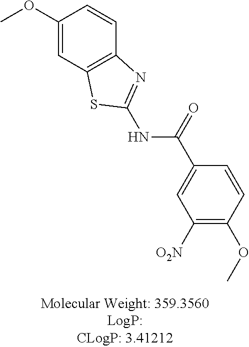 [SMILES: COc1ccc2nc(NC(=O)c3ccc(OC)c([N+](=O)[O-])c3)sc2c1] | 359 | ND-39 |
| 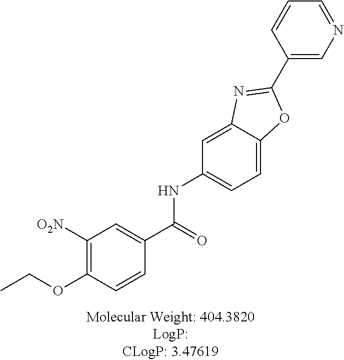 [SMILES: CCOc1ccc(C(=O)Nc2ccc3oc(-c4cccnc4)nc3c2)cc1[N+](=O)[O-]] | 404 | ND-44 |
| 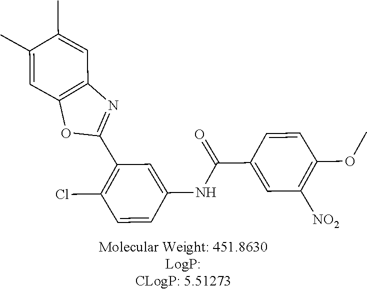 | 451 | ND-45 |
| 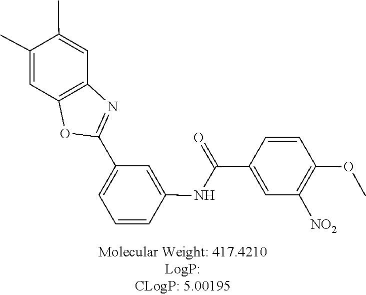 | 417 | ND-46 |
| 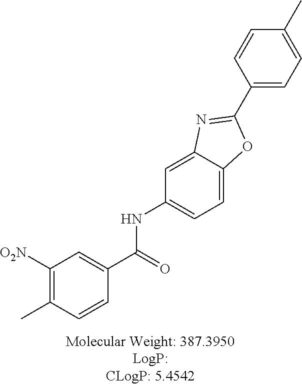 | 387 | ND-49 |
| 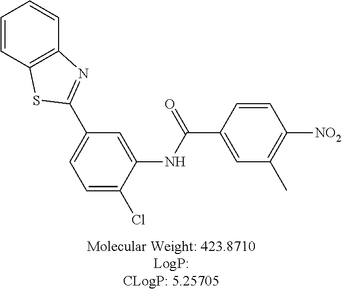 [SMILES: Cc1cc(C(=O)Nc2cc(-c3nc4ccccc4s3)ccc2Cl)ccc1[N+](=O)[O-]] | 423 | ND-51 |
| 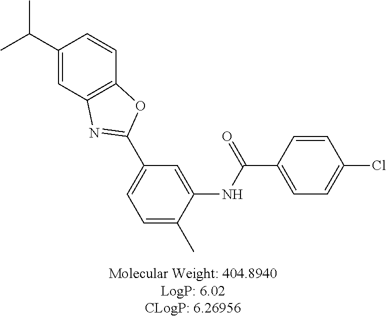 [SMILES: Cc1ccc(-c2nc3cc(C(C)C)ccc3o2)cc1NC(=O)c1ccc(Cl)cc1] | 404 | ND-53 |
| 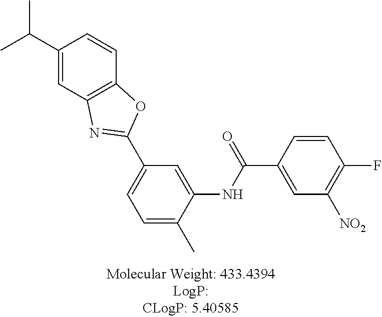 [SMILES: Cc1ccc(-c2nc3cc(C(C)C)ccc3o2)cc1NC(=O)c1ccc(F)c([N+](=O)[O-])c1] | 433 | ND-54 |
| 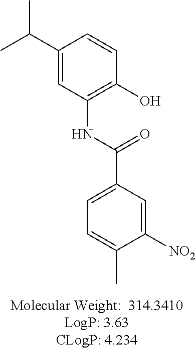 [SMILES: Cc1ccc(C(=O)Nc2cc(C(C)C)ccc2O)cc1[N+](=O)[O-]] | 314 | ND-55 |
| 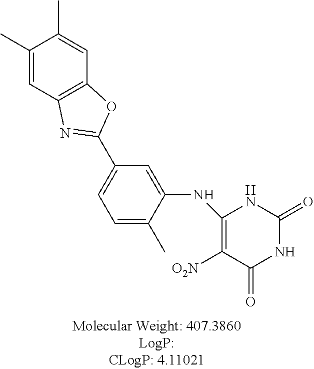 [SMILES: Cc1cc2nc(-c3ccc(C)c(Nc4[nH]c(=O)[nH]c(=O)c4[N+](=O)[O-])c3)oc2cc1C] | 407 | ND-56 |
| 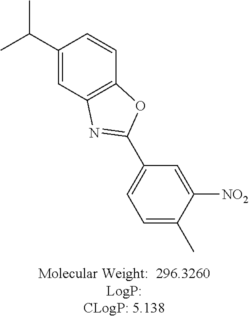 [SMILES: Cc1ccc(-c2nc3cc(C(C)C)ccc3o2)cc1[N+](=O)[O-]] | 296 | ND-57 |
| 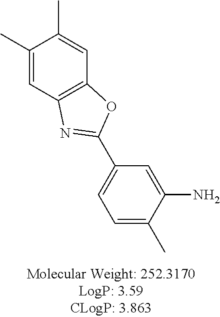 [SMILES: Cc1cc2nc(-c3ccc(C)c(N)c3)oc2cc1C] | 252 | ND-59 |
| 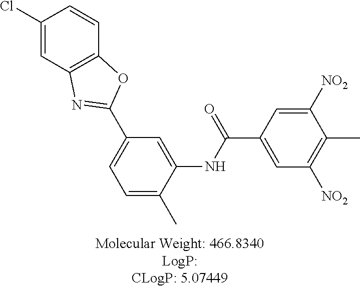 [SMILES: Cc1ccc(-c2nc3cc(Cl)ccc3o2)cc1NC(=O)c1cc([N+](=O)[O-])c(C)c([N+](=O)[O-])c1] | 467 | ND-61 |
| 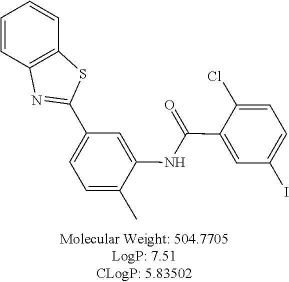 [SMILES: Cc1ccc(-c2nc3ccccc3s2)cc1NC(=O)c1cc(I)ccc1Cl] | 505 | ND-62 |
| 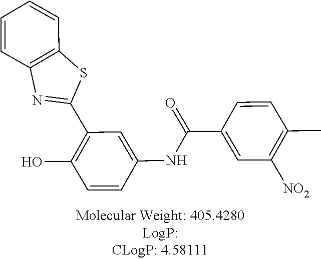 [SMILES: Cc1ccc(C(=O)Nc2ccc(O)c(-c3nc4ccccc4s3)c2)cc1[N+](=O)[O-]] | 405 | ND-63 |
| 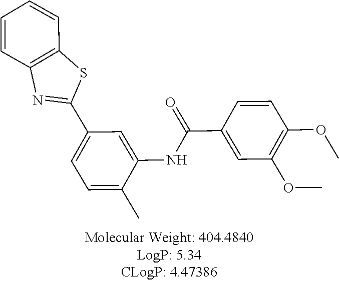 [SMILES: COc1ccc(C(=O)Nc2cc(-c3nc4ccccc4s3)ccc2C)cc1OC] | 404 | ND-66 |
| 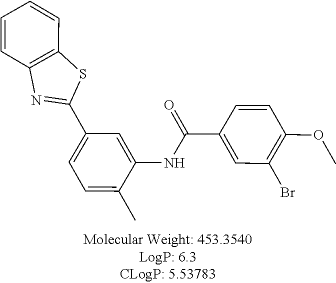 [SMILES: COc1ccc(C(=O)Nc2cc(-c3nc4ccccc4s3)ccc2C)cc1Br] | 453 | ND-68 |
| 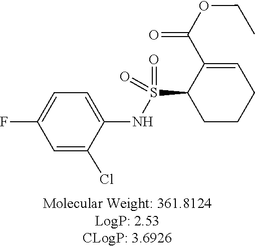 [SMILES: CCOC(=O)C1=CCCC[C@H]1S(=O)(=O)Nc1ccc(F)cc1Cl] | 361 | ND-72 |
| 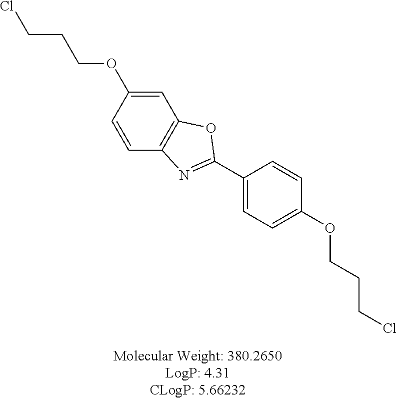 [SMILES: ClCCCOc1ccc(-c2nc3ccc(OCCCCl)cc3o2)cc1] | 380 | ND-73 |
| 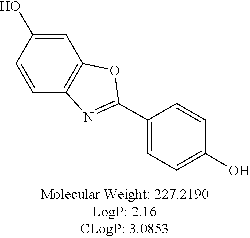 [SMILES: Oc1ccc(-c2nc3ccc(O)cc3o2)cc1] | 227 | ND-74 |
| 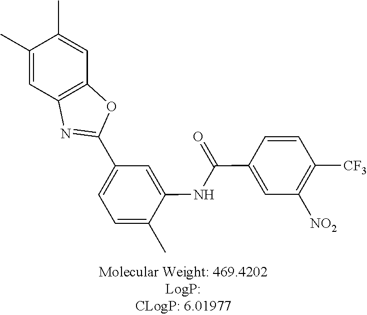 [SMILES: Cc1cc2nc(-c3ccc(C)c(NC(=O)c4ccc(C(F)(F)F)c([N+](=O)[O-])c4)c3)oc2cc1C] | 469 | ND-75 |
| 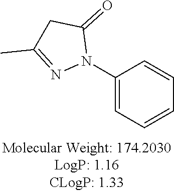 | 174 | ND-77 |
| 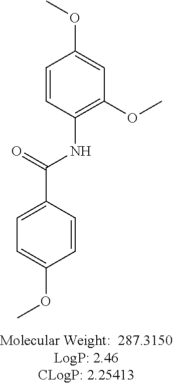 [SMILES: COc1ccc(C(=O)Nc2ccc(OC)cc2OC)cc1] | 287 | ND-78 |
| 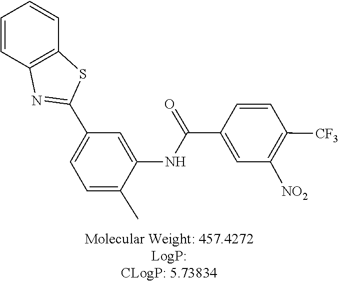 [SMILES: Cc1ccc(-c2nc3ccccc3s2)cc1NC(=O)c1ccc(C(F)(F)F)c([N+](=O)[O-])c1] | 457 | ND-79 |
| 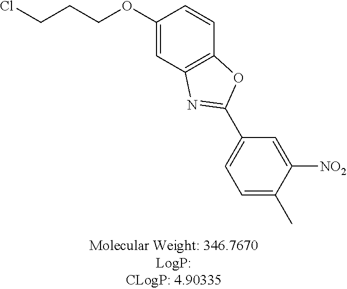 [SMILES: Cc1ccc(-c2nc3cc(OCCCCl)ccc3o2)cc1[N+](=O)[O-]] | 346 | ND-81 |
| 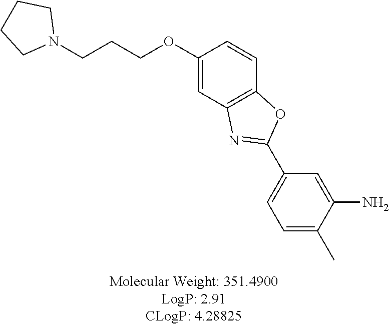 [SMILES: Cc1ccc(-c2nc3cc(OCCCN4CCCC4)ccc3o2)cc1N] | 351 | ND-82 |
| 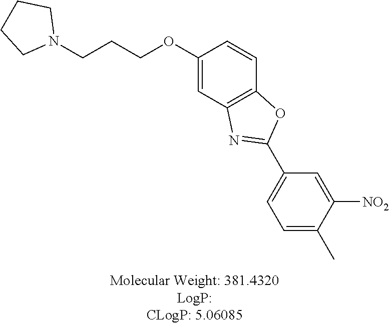 [SMILES: Cc1ccc(-c2nc3cc(OCCCN4CCCC4)ccc3o2)cc1[N+](=O)[O-]] | 404 | ND-83 |
| 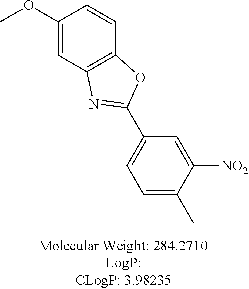 [SMILES: COc1ccc2oc(-c3ccc(C)c([N+](=O)[O-])c3)nc2c1] | 284 | ND-84 |
| 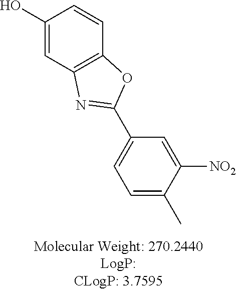 [SMILES: Cc1ccc(-c2nc3cc(O)ccc3o2)cc1[N+](=O)[O-]] | 270 | ND-85 |
| 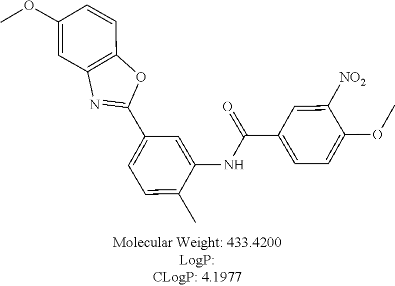 | 433 | ND-86 |
| 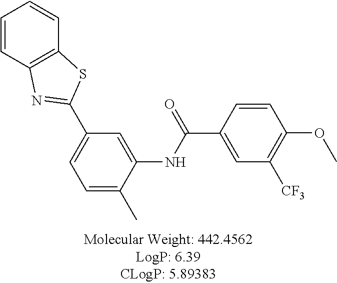 [SMILES: COc1ccc(C(=O)Nc2cc(-c3nc4ccccc4s3)ccc2C)cc1C(F)(F)F] | 442 | ND-87 |
| 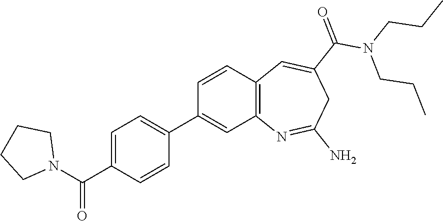 [SMILES: CCCN(CCC)C(=O)C1=Cc2ccc(-c3ccc(C(=O)N4CCCC4)cc3)cc2N=C(N)C1] | 458 | ND-88 |
| 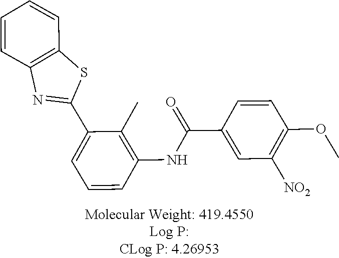 [SMILES: COc1ccc(C(=O)Nc2cccc(-c3nc4ccccc4s3)c2C)cc1[N+](=O)[O-]] | 419 | ND-90 |
| 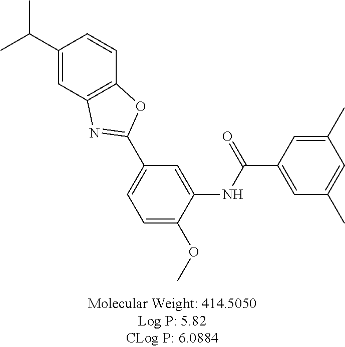 [SMILES: COc1ccc(-c2nc3cc(C(C)C)ccc3o2)cc1NC(=O)c1cc(C)cc(C)c1] | 414 | ND-91 |
| 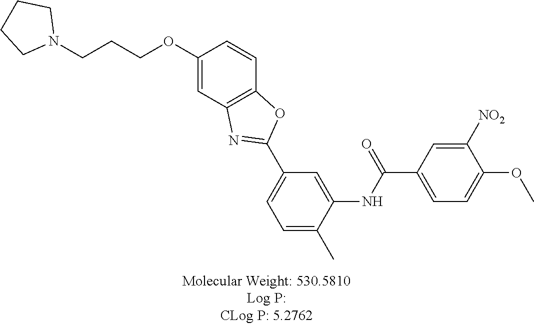 [SMILES: COc1ccc(C(=O)Nc2cc(-c3nc4cc(OCCCN5CCCC5)ccc4o3)ccc2C)cc1[N+](=O)[O-]] | 530 | ND-92 |
| 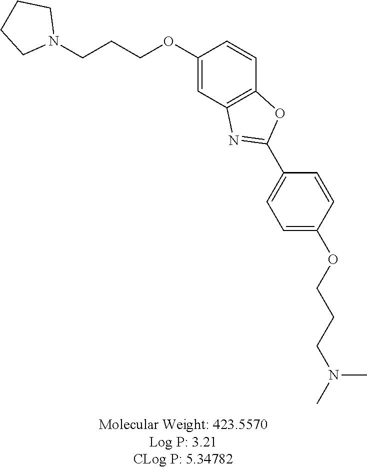 [SMILES: CN(C)CCCOc1ccc(-c2nc3cc(OCCCN4CCCC4)ccc3o2)cc1] | 423 | ND-93 |
| 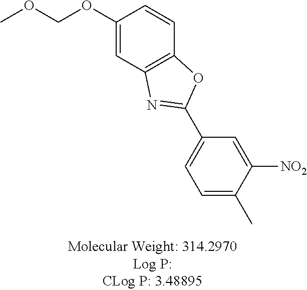 [SMILES: COCOc1ccc2oc(-c3ccc(C)c([N+](=O)[O-])c3)nc2c1] | 314 | ND-96 |
| 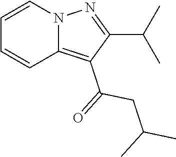 [SMILES: CC(C)CC(=O)c1c(C(C)C)nn2ccccc12] | 244 | ND-97 |
| 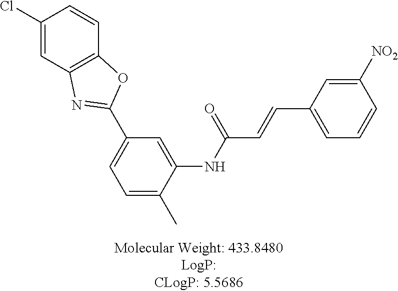 [SMILES: Cc1ccc(-c2nc3cc(Cl)ccc3o2)cc1NC(=O)/C=C/c1cccc([N+](=O)[O-])c1] | 433 | ND-99 |
| 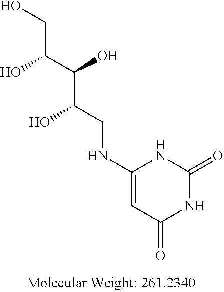 [SMILES: O=c1cc(NC[C@H](O)[C@H](O)[C@H](O)CO)[nH]c(=O)[nH]1] | 261 | ND-100 |
| 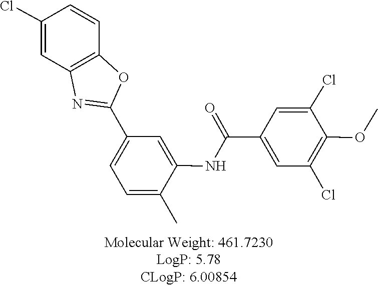 [SMILES: COc1c(Cl)cc(C(=O)Nc2cc(-c3nc4cc(Cl)ccc4o3)ccc2C)cc1Cl] | 461 | ND-101 |
| 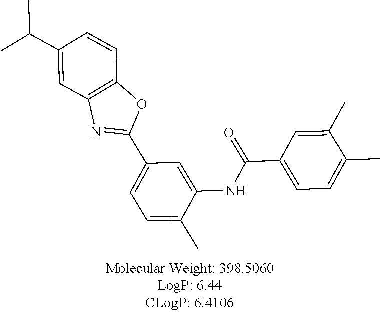 [SMILES: Cc1ccc(C(=O)Nc2cc(-c3nc4cc(C(C)C)ccc4o3)ccc2C)cc1C] | 398 | ND-103 |
| 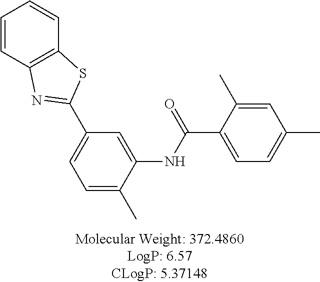 [SMILES: Cc1ccc(C(=O)Nc2cc(-c3nc4ccccc4s3)ccc2C)c(C)c1] | 372 | ND-104 |
| 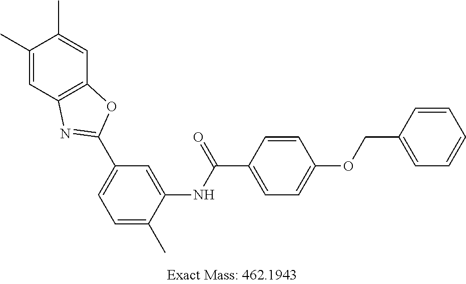 [SMILES: Cc1cc2nc(-c3ccc(C)c(NC(=O)c4ccc(OCc5ccccc5)cc4)c3)oc2cc1C] | 462 | ND-105 |
| 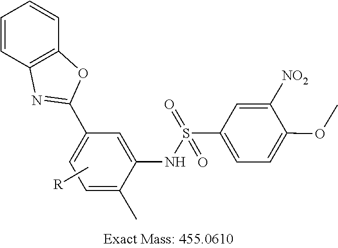 | 455 | ND-106 |
| 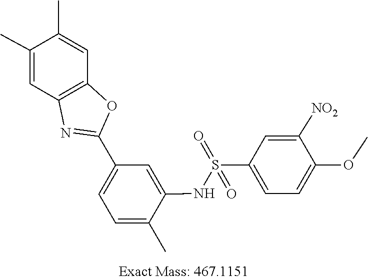 | 467 | ND-107 |
| 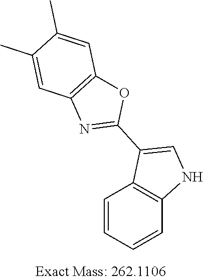 [SMILES: Cc1cc2nc(-c3c[nH]c4ccccc34)oc2cc1C] | 262 | ND-108 |
| 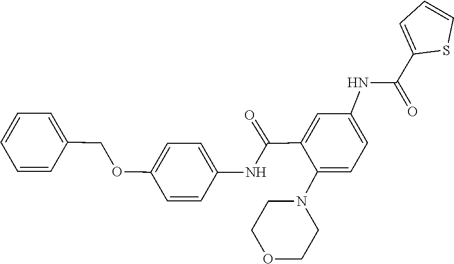 [SMILES: O=C(Nc1ccc(N2CCOCC2)c(C(=O)Nc2ccc(OCc3ccccc3)cc2)c1)c1cccs1] | 513 | NB-18 |
| 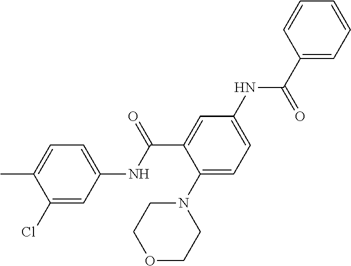 [SMILES: Cc1ccc(NC(=O)c2cc(NC(=O)c3ccccc3)ccc2N2CCOCC2)cc1Cl] | 450 | NB-1 |
| 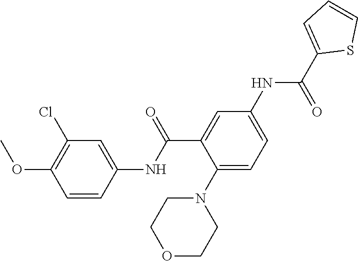 [SMILES: COc1ccc(NC(=O)c2cc(NC(=O)c3cccs3)ccc2N2CCOCC2)cc1Cl] | 471 | NB-22 |
| 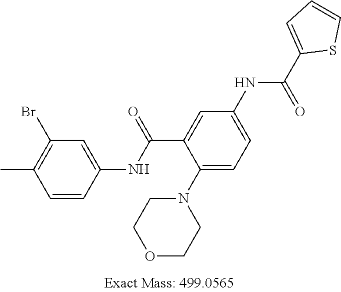 [SMILES: Cc1ccc(NC(=O)c2cc(NC(=O)c3cccs3)ccc2N2CCOCC2)cc1Br] | 499 | NB-23 |
| 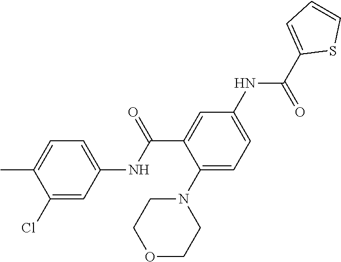 [SMILES: Cc1ccc(NC(=O)c2cc(NC(=O)c3cccs3)ccc2N2CCOCC2)cc1Cl] | 456 | NB-8 |
| 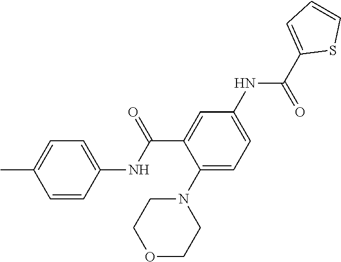 [SMILES: Cc1ccc(NC(=O)c2cc(NC(=O)c3cccs3)ccc2N2CCOCC2)cc1] | 422 | NB-17 |
| 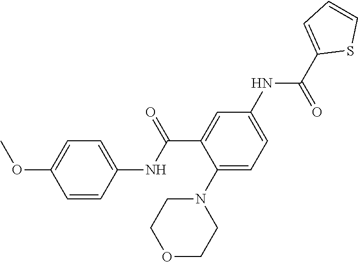 [SMILES: COc1ccc(NC(=O)c2cc(NC(=O)c3cccs3)ccc2N2CCOCC2)cc1] | 438 | NB-14 |
| 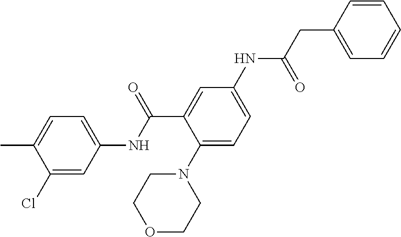 [SMILES: Cc1ccc(NC(=O)c2cc(NC(=O)Cc3ccccc3)ccc2N2CCOCC2)cc1Cl] | 464 | NB-3 |
| 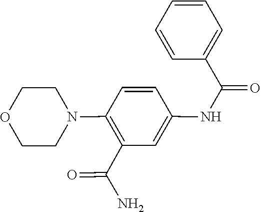 [SMILES: NC(=O)c1cc(NC(=O)c2ccccc2)ccc1N1CCOCC1] | 325.4 | NB-4 |

### IV. Methods of Use

i. Methods of Altering Activity and/or Expression of Interferon Regulatory Factors (IRFs).

IRFs have been shown to be involved in mediating a neuroprotective effect of preconditioning agents. For example, neuroprotective benefits of preconditioning with the preconditioning agent LPS has been shown to be mediated at least in party by IRF3 and IRF7. By use of a high throughput screening methodology that relied on two reporter constructs, one for IRF-specific activity and the other for NF-κB, molecules were identified which have high IRF modulatory activity and corresponding low NF-κB modulatory activity. Subsequent structure activity relationship (SAR) approaches resulted compounds with improved properties in terms of IRF modulatory activity.

In one particular example, methods are disclosed for altering activity and/or expression of an IRF in a subject suffering from a condition or disease, comprising administering to the subject an effective amount of an IRF modulatory agent, the IRF modulatory agent comprising one or more compounds of Table 7 and/or Table 8, or derivatives thereof. For example, the IRF modulatory agent may comprise a compound or compounds of the formulas illustrated at Tables 1-6. The IRF modulatory agent may be an analog/derivative of the disclosed agents which may be designed and synthesized according to the chemical principles known to one of ordinary skill in the art and identified as an IRF modulatory agent by methods known to those of ordinary skill in the art, including the 8-point dose response assay of Example 20.

The disclosed IRF modulatory agents can alter the expression of nucleic acid sequences (e.g., DNA, cDNA, mRNA, etc.) and proteins of the IRF family, including but not limited to IRF3 and IRF7. An increase in expression or activity does not need to be 100% for the agent to be effective. For example, the agent can increase the expression or biological activity by a desired amount, for example by at least 5%, at least 10%, at least 20%, at least 30%, at least 50%, at least 60%, at least 70%, at least 80%, at least 90%, at least 95%, at least 98%, or even at least 100%, including about 15% to about 98%, about 30% to about 95%, about 40% to about 80%, about 50% to about 70%, as compared to activity or expression in a control. Methods of assessing IRF expression and activity are known to those of ordinary skill in the art, including those described in the Examples below (e.g., ELISA with commercially available antibodies, RT-PCR, western blot analysis, microarray analysis, etc.).

In a particular example, the subject is a human.

In additional aspects, the method includes selecting the subject suffering from the condition or disease. Such selecting may include diagnosing the subject with the condition or disease prior to administering the effective amount of the IRF modulatory agent to the subject. As one particular example, such selecting includes identifying the patient as suffering from a stroke.

In some examples, following measurement of expression and/or activity levels of IRF(s), assay results, findings, diagnoses, predictions and/or treatment recommendations may be recorded and communicated to technicians, physicians and/or patients, for example. In certain embodiments, computers are used to communicate such information to interested parties, such as patients and/or the attending physicians.

In one embodiment, the results and/or related information may be communicated to the subject by the subject's treating physician. Alternatively, the results may be communicated directly to a test subject by any means of communication, including writing, such as by providing a written report, electronic forms of communication, such as email, or telephone. Communication may be facilitated by use of a computer, such as in the case of email communications. In certain embodiments, the communication containing results of a diagnostic test and/or conclusions drawn from and/or treatment recommendations based on the test, may be generated and delivered automatically to the subject using a combination of computer hardware and software which will be familiar to artisans skilled in telecommunications. In certain embodiments of the methods of the disclosure, all or some of the method steps, including assaying of samples, diagnosing of diseases, and communicating of assay results or diagnoses, may be carried out in diverse (e.g., foreign) jurisdictions.

In several embodiments, identification of the subject as being afflicted with a stroke results in the physician treating the subject, such as prescribing one or more of the disclosed IRF modulatory agents for inhibiting or delaying one or more signs or symptoms associated with the stroke. However, as discussed herein, it may be understood that selecting the subject suffering from the condition or disease is not limited to stroke, but equally apply to conditions and/or diseases including but not limited to chronic fatigue syndrome, multiple sclerosis, viral infection, and cancer. Similar to that discussed above for stroke conditions, identification of the subject as being afflicted with one of chronic fatigue syndrome, multiple sclerosis, viral infection, and cancer may result in the physician treating the subject by, for example, prescribing one or more of the disclosed IRF modulatory agents for inhibiting or delaying one or more signs or symptoms associated with the condition or disease.

In particular embodiments, the one or more disclosed IRF modulatory agents are selected from four families of molecules, termed herein the NA chemofamily, the NC chemofamily, the NB chemofamily and the ND chemofamily. Exemplary molecules from the NA chemofamily for upregulating IRFs include but are not limited to 4-((3-ethoxybenzyl)oxy)-N-(4-(4-(thiophene-2-carbonyl)piperazin-1-yl)phenyl)benzamide (NA-42), 4-((3-methoxybenzyl)oxy)-N-(4-(4-(thiophene-2-carbonyl)piperazin-1-yl)phenyl)benzamide (NA-24) and 5-bromo-N-{4-[4-(2-thienylcarbonyl)-1-piperazinyl]phenyl}-1-naphthamide (NA-21). Exemplary molecules from the ND chemofamily include but are not limited to N-(5-(5,6-dimethylbenzo[d]oxazol-2-yl)-2-methylphenyl)-4-methoxy-3-nitrobenzamide (ND-13), 4-methoxy-N-(5-(5-(methoxymethoxy)benzo[d]oxazol-2-yl)-2-methylphenyl)-3-nitrobenzamide (ND-95), and N-[5-(1,3-benzothiazol-2-yl)-2-methylphenyl]-4-methoxy-3-nitrobenzamide (ND-52). Exemplary molecules from the NB chemofamily include but are not limited to NB-18, NB-1, NB-22, NB-23, NB-8, NB-17, NB-14, NB-3 and NB-4. Exemplary molecules from the NC chemofamily include but are not limited to NC-1, NC-2, NC-3 and NC-4.

ii. Methods of Treating a Subject Having a Condition/Disorder or Disease that is at Least in Part Regulated by Activity and/or Expression of an IRF.

Also disclosed are methods of treating a subject with a condition/disorder or disease that is at least partially regulated by IRF activity and/or expression, by contacting a cell or cells of the subject with an effective amount of an IRF modulatory agent comprising one or more compounds encompassed by Table 7 and/or Table 8, or analogs/derivatives thereof, thereby treating the condition/disorder or disease. For example, the IRF modulatory agent may comprise a compound or compounds of the formulas illustrated at Tables 1-6. The IRF modulatory agent may be an analog/derivative of the disclosed agents which may be designed and synthesized according to the chemical principles known to one of ordinary skill in the art and identified as an IRF modulatory agent by methods known to those of ordinary skill in the art, including the 8-point dose response assay of Example 20.

The disclosed IRF modulatory agents can alter the expression of nucleic acid sequences (e.g., DNA, cDNA, mRNA, etc.) and proteins of the IRF family, including but not limited to IRF3 and IRF7. An increase in expression or activity does not need to be 100% for the agent to be effective. For example, the agent can increase the expression or biological activity by a desired amount, for example by at least 5%, at least 10%, at least 20%, at least 30%, at least 50%, at least 60%, at least 70%, at least 80%, at least 90%, at least 95%, at least 98%, or even at least 100%, including about 15% to about 98%, about 30% to about 95%, about 40% to about 80%, about 50% to about 70%, as compared to activity or expression in a control. Methods of assessing IRF expression and activity are known to those of ordinary skill in the art, including those described in the Examples below (e.g., ELISA with commercially available antibodies, RT-PCR, western blot analysis, microarray analysis, etc.).

In a particular example, the subject is a human.

In additional aspects, the method includes selecting the subject suffering from the condition or disease. Such selecting may include diagnosing the subject with the condition or disease prior to administering the effective amount of the IRF modulatory agent to the subject. As one particular example, such selecting includes identifying the patient as suffering from a stroke. Methods for diagnosing and selecting a subject having a condition/disorder or disease that is at least in part regulated by activity and/or expression of an IRF can include those provided herein (including those in the Methods of altering activity and/or expression of interferon regulatory factors (IRFs)). In some examples, such selecting can include the selection of subjects based on their lifestyle (e.g., engaged in moderate to intense exercise or physical activities), age (e.g., elderly population at more risk of experiencing a condition or disease including but not limited to stroke, cancer, multiple sclerosis, etc.), or predisposition to the condition or disease.

In an example of stroke, in one particular embodiment of the method, contacting the cell or cells of the subject with the IRF modulatory agent delays a depletion of cellular energy stores and delays membrane potential depolarization of the cell or cells affected by the stroke as compared to a rate at which depletion of cellular energy stores and membrane potential depolarization occurs in the absence of the cell or cells being contacted with the IRF modulatory agent.

iii. Methods of increasing a time frame in which one or more treatments can be effectively provided to a subject suffering from an acute ischemic event.

Also disclosed herein are methods which increase a time frame in which one or more treatments can be effectively provided to a subject suffering from an acute ischemic event. In some embodiments, increasing the time frame is via altering activity and/or expression of an IRF by administering to the subject an IRF modulatory agent within a predetermined period of time of an initiation of the acute ischemic event. The IRF modulatory agent may be selected from any one or more of the compounds encompassed by Table 7 and Table 8, or analogs/derivatives thereof. For example, the IRF modulatory agent may comprise a compound or compounds of the formulas illustrated at Tables 1-6. In particular embodiments, the IRF modulatory agent or agents may include one or more of 4-((3-ethoxybenzyl)oxy)-N-(4-(4-(thiophene-2-carbonyl)piperazin-1-yl)phenyl)benzamide (NA-42), 4-((3-methoxybenzyl)oxy)-N-(4-(4-(thiophene-2-carbonyl)piperazin-1-yl)phenyl)benzamide (NA-24) and 5-bromo-N-{4-[4-(2-thienylcarbonyl)-1-piperazinyl]phenyl}-1-naphthamide (NA-21). Exemplary molecules from the ND chemofamily include but are not limited to N-(5-(5,6-dimethylbenzo[d]oxazol-2-yl)-2-methylphenyl)-4-methoxy-3-nitrobenzamide (ND-13), 4-methoxy-N-(5-(5-(methoxymethoxy)benzo[d]oxazol-2-yl)-2-methylphenyl)-3-nitrobenzamide (ND-95), and N-[5-(1,3-benzothiazol-2-yl)-2-methylphenyl]-4-methoxy-3-nitrobenzamide (ND-52). Exemplary molecules from the NB chemofamily include, but are not limited to NB-18, NB-1, NB-22, NB-23, NB-8, NB-17, NB-14, NB-3 and NB-4. Exemplary molecules from the NC chemofamily include but are not limited to NC-1, NC-2, NC-3 and NC-4. Similar to that discussed above, it may be understood that the IRF modulatory agent or agents may comprise a derivative/analog of any of the disclosed IRF modulatory agents discussed herein, which may be designed and synthesized according to the chemical principles known to those of ordinary skill in the art and identified as IRF modulatory agents, for example by use of the 8-point dose response assay of Example 20. In some examples, the IRF modulatory agent may comprise a compound or compounds of one or more of the formulas depicted at Tables 1-6.

In some examples, the IRF is at least IRF3 and/or IRF7. The IRF modulatory agent may increase activity and/or expression of the IRF as compared to activity and/or expression of the IRF prior to or in an absence of administration of the IRF modulatory agent.

The disclosed IRF modulatory agents can alter the expression of nucleic acid sequences (e.g., DNA, cDNA, mRNA, etc.) and proteins of the IRF family, including but not limited to IRF3 and IRF7. An increase in expression or activity does not need to be 100% for the agent to be effective. For example, the agent can increase the expression or biological activity by a desired amount, for example by at least 5%, at least 10%, at least 20%, at least 30%, at least 50%, at least 60%, at least 70%, at least 80%, at least 90%, at least 95%, at least 98%, or even at least 100%, including about 15% to about 98%, about 30% to about 95%, about 40% to about 80%, about 50% to about 70%, as compared to activity or expression in a control. Methods of assessing IRF expression and activity are known to those of ordinary skill in the art, including those described in the Examples below (e.g., ELISA with commercially available antibodies, RT-PCR, western blot analysis, microarray analysis, etc.).

In additional aspects, the one or more treatments include administration of a thrombolytic agent and/or performing a mechanical thrombectomy. The one or more treatments may be administered/performed at a time after administering the IRF modulatory agent, and before the time frame of the therapeutic window elapses. Increasing the time frame may comprise improving a tolerance of neural tissue to the acute ischemic event in a manner that delays a depletion of cellular energy stores and delays membrane potential depolarization of the neural tissue affected by the acute ischemic event as compared to a rate at which depletion of cellular energy stores and membrane potential depolarization occurs in the absence of administering to the subject the IRF modulatory agent. This may result in a reduction or inhibition of neuronal death over time, thereby increasing the time in which the one or more treatments may be given to the subject and still be effective. For example, in the absence of the subject being administered the IRF modulatory agent or agents, there may be less time in which the treatments can be effectively used to effect a reduction in one or more signs or symptoms associated with the acute ischemic event.

In additional aspects, the method includes selecting the subject suffering from the acute ischemic event. Such selecting may include diagnosing the subject as experiencing the acute ischemic event prior to administering the effective amount of the IRF modulatory agent to the subject. Methods for diagnosing and selecting a subject having a condition/disorder or disease that is at least in part regulated by activity and/or expression of an IRF can include those provided herein (including those in the Methods of altering activity and/or expression of interferon regulatory factors (IRFs), and the Methods of treating a subject having a condition/disorder or disease that is at least in part regulated by activity and/or expression of an IRF). In some examples, such selecting can include the selection of subjects based at least in part on their lifestyle (e.g., engaged in moderate to intense exercise or physical activities), age (e.g., elderly population at more risk of experiencing an acute ischemic event), or predisposition to the condition or disease. In still further examples, such selecting may include but is not limited to questioning of the subject or family member about symptoms and medical history, performing of a physical exam (e.g., checking blood pressure, checking for mental alertness, numbness, weakness, trouble speaking, trouble seeing, trouble walking, etc.), conducting a neurological exam to show how well the subject's nervous system is working. In still further examples, such selecting may include but is not limited to imaging tests. Such imaging tests may include but are not limited to computed tomography (CT) scan, magnetic resonance imaging (MRI) scan, CT or MR angiogram, carotid ultrasound, trans-cranial doppler (TCD) ultrasound, electroencephalogram (EEG), electrocardiogram (ECG or EKG), etc. In still further examples, such selecting may include but is not limited to blood tests which may help ascertain the cause of the stroke, which in some examples may be used to determine IRF modulatory agent dosing, timing, etc. Such blood tests may include complete blood count (CBC), serum electrolytes, blood clotting tests, heart attach tests, thyroid tests, blood glucose tests, cholesterol tests, C-reactive protein test and blood protein test, etc. It may be understood that any one or more such testing procedures for stroke may be used in any one of the methods herein described without departing from the scope of the present disclosure.

### V. Administration of an Effective Amount of an IRF (e.g., IRF3) Modulatory Agent

For any of the disclosed methods, an effective amount of the IRF modulatory agent is one when administered by a particular route and concentration induces the desired response, which may include reducing or inhibiting one or more signs or symptoms associated with a condition or disease including but not limited to stroke, cancer, chronic fatigue syndrome, multiple sclerosis, and viral infection.

i. Administration Routes, Formulations and Concentrations

Methods of administration of the disclosed IRF modulatory agents are routine, and can be determined by a skilled clinician. The disclosed IRF modulatory agents or other therapeutic substance are in general administered topically, nasally, intravenously, orally, intracranially, intramuscularly, parenterally or as implants, but even rectal or vaginal use is possible in principle. The disclosed IRF modulatory agents also may be administered to a subject using a combination of these techniques.

Suitable solid or liquid pharmaceutical preparation forms are, for example, aerosols, (micro)capsules, creams, drops, drops or injectable solution in ampoule form, emulsions, granules, powders, suppositories, suspensions, syrups, tablets, coated tablets, and also preparations with protracted release of active compounds, in whose preparation excipients and additives and/or auxiliaries such as binders, coating agents, disintegrants, flavorings, lubricants, solubilizers, sweeteners, or swelling agents are customarily used as described above. The pharmaceutical agents are suitable for use in a variety of drug delivery systems. For a brief review of various methods for drug delivery, see Langer, “New Methods of Drug Delivery,” Science 249:1527-1533 (1990), incorporated by reference herein to the extent not inconsistent with the present disclosure.

The disclosed IRF modulatory agents or other therapeutic agents of the present disclosure can be formulated into therapeutically-active pharmaceutical agents that can be administered to a subject parenterally or orally. Parenteral administration routes include, but are not limited to epidermal, intraarterial, intramuscular (IM and depot IM), intraperitoneal (IP), intravenous (IV), intrasternal injection or infusion techniques, intranasal (inhalation), intrathecal, injection into the stomach, subcutaneous injections (subcutaneous (SQ and depot SQ), transdermal, topical, and ophthalmic.

The disclosed IRF modulatory agents or other therapeutic agents can be mixed or combined with a suitable pharmaceutically acceptable excipients to prepare pharmaceutical agents. Pharmaceutically acceptable excipients include, but are not limited to, alumina, aluminum stearate, buffers (such as phosphates), glycine, ion exchangers (such as to help control release of charged substances), lecithin, partial glyceride mixtures of saturated vegetable fatty acids, potassium sorbate, serum proteins (such as human serum albumin), sorbic acid, water, salts or electrolytes such as cellulose-based substances, colloidal silica, disodium hydrogen phosphate, magnesium trisilicate, polyacrylates, polyalkylene glycols, such as polyethylene glycol, polyethylene-polyoxypropylene-block polymers, polyvinyl pyrrolidone, potassium hydrogen phosphate, protamine sulfate, group 1 halide salts such as sodium chloride, sodium carboxymethylcellulose, waxes, wool fat, and zinc salts, for example. Liposomal suspensions may also be suitable as pharmaceutically acceptable carriers.

Upon mixing or addition of one or more disclosed IRF modulatory agents and/or or other therapeutic agents, the resulting mixture may be a solid, solution, suspension, emulsion, or the like. These may be prepared according to methods known to those of ordinary skill in the art. The form of the resulting mixture depends upon a number of factors, including the intended mode of administration and the solubility of the agent in the selected carrier. Pharmaceutical carriers suitable for administration of the disclosed IRF modulatory agents or other therapeutic agents include any such carriers known to be suitable for the particular mode of administration. In addition, the disclosed IRF modulatory agents or other therapeutic substance can also be mixed with other inactive or active materials that do not impair the desired action, or with materials that supplement the desired action, or have another action.

Methods for solubilizing may be used where the agents exhibit insufficient solubility in a carrier. Such methods are known and include, but are not limited to, dissolution in aqueous sodium bicarbonate, using cosolvents such as dimethylsulfoxide (DMSO), and using surfactants such as TWEEN® (ICI Americas, Inc., Wilmington, Del.).

The disclosed IRF modulatory agents or other therapeutic agents can be prepared with carriers that protect them against rapid elimination from the body, such as coatings or time-release formulations. Such carriers include controlled release formulations, such as, but not limited to, microencapsulated delivery systems. A disclosed IRF modulatory agents or other therapeutic agent is included in the pharmaceutically acceptable carrier in an amount sufficient to exert a therapeutically useful effect, typically in an amount to avoid undesired side effects, on the treated subject. The therapeutically effective concentration may be determined empirically by testing the compounds in known in vitro and in vivo model systems for the treated condition. For example, mouse models of one or more of stroke, cancer, chronic fatigue syndrome, multiple sclerosis, viral infection or immune response to antigen, etc., may be used to determine effective amounts or concentrations that can then be translated to other subjects, such as humans, as known in the art.

Injectable solutions or suspensions can be formulated, using suitable non-toxic, parenterally-acceptable diluents or solvents, such as 1,3-butanediol, isotonic sodium chloride solution, mannitol, Ringer's solution, saline solution, or water; or suitable dispersing or wetting and suspending agents, such as sterile, bland, fixed oils, including synthetic mono- or diglycerides, and fatty acids, including oleic acid; a naturally occurring vegetable oil such as coconut oil, cottonseed oil, peanut oil, sesame oil, and the like; glycerine; polyethylene glycol; propylene glycol; or other synthetic solvent; antimicrobial agents such as benzyl alcohol and methyl parabens; antioxidants such as ascorbic acid and sodium bisulfate; buffers such as acetates, citrates, and phosphates; chelating agents such as ethylenediaminetetraacetic acid (EDTA); agents for the adjustment of tonicity such as sodium chloride and dextrose; and combinations thereof. Parenteral preparations can be enclosed in ampoules, disposable syringes, or multiple dose vials made of glass, plastic, or other suitable material. Buffers, preservatives, antioxidants, and the like can be incorporated as required. Where administered intravenously, suitable carriers include physiological saline, phosphate-buffered saline (PBS), and solutions containing thickening and solubilizing agents such as glucose, polyethylene glycol, polypropyleneglycol, and mixtures thereof. Liposomal suspensions, including tissue-targeted liposomes, may also be suitable as pharmaceutically acceptable carriers.

For topical application, one or more disclosed IRF modulatory agents, or other therapeutic agent may be made up into a cream, lotion, ointment, solution, or suspension in a suitable aqueous or non-aqueous carrier. Topical application can also be accomplished by transdermal patches or bandages which include the therapeutic substance. Additives can also be included, e.g., buffers such as sodium metabisulphite or disodium edetate; preservatives such as bactericidal and fungicidal agents, including phenyl mercuric acetate or nitrate, benzalkonium chloride, or chlorhexidine; and thickening agents, such as hypromellose.

If the disclosed IRF modulatory agent, or other therapeutic agent is administered orally as a suspension, the pharmaceutical agents can be prepared according to techniques well known in the art of pharmaceutical formulation and may contain a suspending agent, such as alginic acid or sodium alginate, bulking agent, such as microcrystalline cellulose, a viscosity enhancer, such as methylcellulose, and sweeteners/flavoring agents. Oral liquid preparations can contain conventional additives such as suspending agents, e.g., gelatin, glucose syrup, hydrogenated edible fats, methyl cellulose, sorbitol, and syrup; emulsifying agents, e.g., acacia, lecithin, or sorbitan monooleate; non-aqueous carriers (including edible oils), e.g., almond oil, fractionated coconut oil, oily esters such as glycerine, propylene glycol, or ethyl alcohol; preservatives such as methyl or propyl p-hydroxybenzoate or sorbic acid; and, if desired, conventional flavoring or coloring agents. When formulated as immediate release tablets, these agents can contain dicalcium phosphate, lactose, magnesium stearate, microcrystalline cellulose, and starch and/or other binders, diluents, disintegrants, excipients, extenders, and lubricants.

If oral administration is desired, one or more disclosed IRF modulatory agents, or other therapeutic substances can be provided in a composition that protects it from the acidic environment of the stomach. For example, the disclosed IRF modulatory agents or other therapeutic agents can be formulated with an enteric coating that maintains its integrity in the stomach and releases the active compound in the intestine. The disclosed IRF modulatory agents, or other therapeutic agent can also be formulated in combination with an antacid or other such ingredient.

Oral compositions generally include an inert diluent or an edible carrier and can be compressed into tablets or enclosed in gelatin capsules. For the purpose of oral therapeutic administration, one or more of the disclosed IRF modulatory agents, or other therapeutic substances can be incorporated with excipients and used in the form of capsules, tablets, or troches. Pharmaceutically compatible adjuvant materials or binding agents can be included as part of the composition.

The capsules, pills, tablets, troches, and the like can contain any of the following ingredients or compounds of a similar nature: a binder such as, but not limited to, acacia, corn starch, gelatin, gum tragacanth, polyvinylpyrrolidone, or sorbitol; a filler such as calcium phosphate, glycine, lactose, microcrystalline cellulose, or starch; a disintegrating agent such as, but not limited to, alginic acid and corn starch; a lubricant such as, but not limited to, magnesium stearate, polyethylene glycol, silica, or talc; a gildant, such as, but not limited to, colloidal silicon dioxide; a sweetening agent such as sucrose or saccharin; disintegrants such as potato starch; dispersing or wetting agents such as sodium lauryl sulfate; and a flavoring agent such as peppermint, methyl salicylate, or fruit flavoring.

When the dosage unit form is a capsule, it can contain, in addition to material of the above type, a liquid carrier, such as a fatty oil. In addition, dosage unit forms can contain various other materials that modify the physical form of the dosage unit, for example, coatings of sugar and other enteric agents. One or more of the disclosed IRF modulatory agents, or other therapeutic agent can also be administered as a component of an elixir, suspension, syrup, wafer, tea, chewing gum, or the like. A syrup may contain, in addition to the active compounds, sucrose or glycerin as a sweetening agent and certain preservatives, dyes and colorings, and flavors.

When administered orally, the compounds can be administered in usual dosage forms for oral administration. These dosage forms include the usual solid unit dosage forms of tablets and capsules as well as liquid dosage forms such as solutions, suspensions, and elixirs. When the solid dosage forms are used, they can be of the sustained release type so that the compounds need to be administered less frequently.

In some implementations, the effective amount of one or more of the disclosed IRF modulatory agents is administered as a single dose per time period, depending on the application. In one example, the single dose per time period may be every three or four months, month, week, or day, or even less such as a single time point or two or more timepoints within a matter of minutes or hours, or it can be divided into at least two unit dosages for administration over a period. Treatment may be continued as long as necessary to achieve the desired results. In some examples, treatment may continue for about 3 or 4 weeks up to about 12-24 months or longer, including ongoing treatment. The compound can also be administered in several doses intermittently, such as every few days (for example, at least about every two, three, four, five, or ten days) or every few weeks (for example at least about every two, three, four, five, or ten weeks).

Particular dosage regimens can be tailored to a particular subject, condition to be treated, or desired result. For example, when the methods of the present disclosure are used to treat stroke or similar conditions, an initial treatment regimen can be applied to arrest the condition and/or increase a therapeutic time window in which other therapeutic approaches may be utilized, including but not limited to administration of tissue plasminogen activator (tPA) and/or use of mechanical thrombectomy. In one example, such initial treatment regimen may include administering a higher dosage of one or more of the disclosed IRF modulatory agents, or administering such material more frequently as compared to later times of treatment. After a desired therapeutic result has been obtained, a second treatment regimen may be applied, such as administering a lower dosage of one or more of the disclosed IRF3 modulatory agents or administering such material less frequently, such as monthly, bi-monthly, quarterly, or semi-annually. In some examples, the second regimen may serve as a “booster”, for example.

Amounts effective for various therapeutic treatments of the present disclosure may, of course, depend on the severity of the disease and the weight and general state of the subject, as well as the absorption, inactivation, and excretion rates of the therapeutically-active compound or component, the dosage schedule, and amount administered, as well as other factors known to those of ordinary skill in the art. It also should be apparent to one of ordinary skill in the art that the exact dosage and frequency of administration will depend on the particular IRF modulatory agent, or other therapeutic substance being administered, the particular condition being treated, the severity of the condition being treated, the age, weight, general physical condition of the particular subject, and other medication the subject may be taking. Typically, dosages used in vitro may provide useful guidance in the amounts useful for in vivo administration of the pharmaceutical composition, and animal models may be used to determine effective dosages for treatment of particular disorders. For example, mouse models of stroke (or other conditions such as cancer, chronic fatigue syndrome, multiple sclerosis, viral infection, etc.) may be used to determine effective dosages that can then be translated to dosage amount for other subjects, such as humans, as known in the art. Various considerations in dosage determination are described, e.g., in Gilman et al., eds., Goodman And Gilman's: The Pharmacological Bases of Therapeutics, 8th ed., Pergamon Press (1990); and Remington's Pharmaceutical Sciences, 17th ed., Mack Publishing Co., Easton, Pa. (1990), each of which is herein incorporated by reference to the extent not inconsistent with the present disclosure.

In specific examples, the one or more disclosed IRF modulatory agents is administered to a subject in an amount sufficient to provide a dose of the agent of between about 10 fmol/g and about 500 nmol/g, such as between about 2 nmol/g and about 20 nmol/g or between about 2 nmol/g and about 10 nmol/g. In additional examples, the IRF3 modulatory agent is administered to a subject in an amount sufficient to provide a dose of between about 0.01 pg/kg and about 1000 mg/kg or between about 0.1 mg/kg and about 1000 mg/kg. In another example, the disclosed IRF3 modulatory agent is administered to a subject in an amount sufficient to provide a dose of agent of between about 0.2 mg/kg and about 2 mg/kg. In further examples, the IRF3 modulatory agent is administered to a subject in an amount sufficient to provide a concentration of IRF3 modulatory agent in the administrated material of between about 5 nM and about 500 nM, such as between about 50 nM and about 200 nm, or about 100 nM. In other examples, the IRF3 modulatory agent is administered to a subject between about 500 μg/ml and about 1 μg/ml, such as about 300 μg/ml and about 3 μg/ml, about 200 μg/ml and about 20 μg/ml, including 500 μg/ml, 400 μg/ml, 300 μg/ml, 250 μg/ml, 200 μg/ml, 150 μg/ml, 100 μg/ml, 50 μg/ml, 25 μg/ml, 12.5 μg/ml, 6.25 μg/ml, 3.125 μg/ml, 2.5 μg/ml and 1.25 μg/ml.

ii. Desired Response

One or more disclosed IRF modulatory agents and/or additional therapeutic agents are administered by a specific route and/or concentration to generate a desired response. In some examples, a desired response refers to an amount effective for lessening, ameliorating, eliminating, preventing, or inhibiting at least one symptom of a disease, disorder, or condition treated and may be empirically determined. In various embodiments of the present disclosure, a desired response is reduction of one or more signs or symptoms associated with one of stroke, cancer, chronic fatigue syndrome, multiple sclerosis, or a viral infection. In another example, in various embodiments of the present disclosure a desired response is an increase in tolerance of neural tissue to an ischemic insult/injury, for example responsive to a stroke. For example, a desired response may be an increased therapeutic window in response to a stroke in which therapies such as use of thombolytics and/or clot retrieval devices can be effectively used to treat a stroke patient. As another example, a desired response is a reduction in damaging inflammation and/or neuronal toxicity to an ischemic insult/injury.

### VI. Clinical Trials

To obtain regulatory approval for the use of one or more of the disclosed IRF modulatory agents to treat a particular condition (e.g., stroke, cancer, chronic fatigue syndrome, multiple sclerosis, viral infection, etc.), clinical trials are performed. As is known in the art, clinical trials progress through phases of testing, which are identified as Phases I, II, III, and IV.

Initially the disclosed IRF modulatory agent is evaluated in a Phase I trial. Typically, Phase I trials are used to determine the best mode of administration (for example, by pill or by injection), the frequency of administration, and the toxicity for the compounds. Phase I studies frequently include laboratory tests, such as blood tests and biopsies, to evaluate the effects of the potential therapeutic in the body of the patient. For a Phase I trial to examine effectiveness of an IRF modulatory agent with regard to stroke, for example, a small group of stroke patients are treated with a specific dose of a disclosed IRF modulatory agent. During the trial, the dose is typically increased group by group in order to determine the maximum tolerated dose (MTD) and the dose-limiting toxicities (DLT) associated with the compound. This process determines an appropriate dose to use in a subsequent Phase II trial.

A Phase II trial can be conducted to further evaluate the effectiveness and safety of the disclosed IRF modulatory agent. I n Phase II trials to examine effectiveness of an IRF modulatory agent with regard to stroke, for example, a disclosed IRF modulatory agent is administered to groups of stroke patients using the dosage found to be effective in Phase I trials.

Phase III trials focus on determining how a disclosed IRF modulatory agent compares to the standard, or most widely accepted, treatment. In Phase III trials, patients are randomly assigned to one of two or more “arms”. In a trial with two arms, for example, one arm will receive the standard treatment (control group) and the other arm will receive a disclosed IRF3 modulatory agent treatment (investigational group).

Phase IV trials are used to further evaluate the long-term safety and effectiveness of a disclosed IRF modulatory agent. Phase IV trials are less common than Phase I, II and III trials and take place after a disclosed IRF modulatory agent has been approved for standard use.

Eligibility of Patients for Clinical Trials

Participant eligibility criteria can range from general (for example, age, sex, type of disease) to specific (for example, type and number of prior treatments, disease characteristics, blood cell counts, organ function). In one embodiment with regard to stroke treatment, eligible patients have been assessed as being of a particular risk for stroke. Eligibility criteria may also vary with trial phase. Patients eligible for clinical trials can also be chosen based on objective measurement and/or assessment. For example, in Phase I and II trials, the criteria often exclude patients who may be at risk from the investigational treatment because of abnormal organ function or other factors. In Phase II and III trials additional criteria are often included regarding disease type and stage, and number and type of prior treatments.

Phase I trials usually include 15 to 30 participants, and Phase II trials typically include up to 100 participants. Phase III trials usually include hundreds to thousands of participants. This large number of participants is necessary in order to determine whether there are true differences between the effectiveness of a disclosed IRF modulatory agent and the standard treatment.

One skilled in the art will appreciate that clinical trials should be designed to be as inclusive as possible without making the study population too diverse to determine whether the treatment might be as effective on a more narrowly defined population. The more diverse the population included in the trial, the more applicable the results could be to the general population, particularly in Phase III trials. Selection of appropriate participants in each phase of clinical trial is considered to be within the ordinary skills of a worker in the art.

Considerations for Designing Phase II Clinical Stroke Trials

Considerations with regard to Phase II acute stroke trials should include but are not limited to route of administration, dose range, duration of treatment, time from stroke onset to initiation of treatment (a key variable), pharmacokinetic profile, side effects and their frequency (with attention to side effect management), interactions with other commonly used medications, drug distribution to the proposed site of action, refinement and identification of the target population (e.g., drugs without preclinical evidence of activity in white matter ischemia should not be studied in patients with subcortical stroke or even large cortical events with attendant subcortical injury), and the obtaining of evidence measurements of therapeutic activity by evaluation of clinical and/or surrogate markers (hints of potential effectiveness).

Considerations for Designing Phase III Clinical Stroke Trials

Considerations for designing Phase III acute stroke trials should include but are not limited to dose selection based on preclinical and phases I and II data, time window for initiation of drug, patient selection based on mechanisms of action, outcome measures (e.g., one type of primary outcome or global assessment), severity of stroke population to be studied, length of follow-up period, use of surrogate markers to provide support of drug efficacy, prespecification of covartate analysis, and fostering of appropriate and effective relationships between sponsors, academicians, and investigators.

Administration of a Disclosed IRF Modulatory Agent in Clinical Trials

As one example, a disclosed IRF modulatory agent may be administered to the trial participants orally. A range of doses of the agent can be tested. Provided with information from preclinical testing, a skilled practitioner can readily determine appropriate dosages of agent for use in clinical trials. In one embodiment, a dose range is from about 100 μg/kg and about 5000 mg/kg of the subject's weight, such as 1 mg/kg and about 2000 mg/kg of the subject's weight, about 100 mg/kg and about 1500 mg/kg of the subject's weight, about 100 μg/kg and about 2000 mg/kg of the subject's weight, about 200 mg/kg and about 1000 mg/kg of the subject's weight, about 200 mg/kg and about 750 mg/kg of the subject's weight, about 250 mg/kg and about 500 mg/kg of the subject's weight, about 100 μm and about 500 mM. In some embodiments, a subject is given a disclosed IRF modulatory agent as an acute treatment (e.g., one time, not repeated). In some embodiments, a subject is given a disclosed IRF modulatory agent more than once (e.g., multiple times), such as for a chronic condition, including, but limited to cancer, or for a vaccine. In some embodiments, subjects are given a disclosed IRF modulatory agent orally at 10 to 60 mg/kg of body weight per day. For example, 10-15 mg/kg of a disclosed IRF modulatory agent is administered for two weeks and if well tolerated the dose is increased by 5-10 mg/kg/week to achieve optimal clinical response. In some examples, the daily dose does not exceed 60 mg/kg of body weight and is given for a minimum of 6 months with liver function monitored every two weeks to monthly.

Pharmacokinetic Monitoring

To fulfill Phase I criteria, distribution of the disclosed IRF modulatory agent may be monitored, for example, by chemical analysis of samples, such as blood, collected at regular intervals. As one example, samples can be taken at regular intervals up until about 72 hours after the start of treatment.

If analysis is not conducted immediately, the samples can be placed on dry ice after collection and subsequently transported to a freezer to be stored at −70° C. until analysis can be conducted. Samples can be prepared for analysis using standard techniques known in the art and the amount of the disclosed IRF modulatory agent present can be determined, for example, by high-performance liquid chromatography (HPLC). Pharmacokinetic data can be generated and analyzed in collaboration with an expert clinical pharmacologist and used to determine, for example, clearance, half-life and maximum plasma concentration.

Monitoring of Patient Outcome

The endpoint of a clinical trial is a measurable outcome that indicates the effectiveness of a compound under evaluation. The endpoint is established prior to the commencement of the trial and will vary depending on the type and phase of the clinical trial.

Types of Endpoints in Acute Stroke Trials

Impairment scales (eg, the NIH Stroke Scales, Canadian Neurological Scale, Scandinavian Stroke Scale, and the European Stroke Scale) may be the most sensitive to change and have the greatest capacity to differentiate between treatment groups, which makes them particularly useful for phase II studies. In addition to measurements of impairment (body dimension), measures reflecting disability (activities dimension) and handicap (participation dimension) should be included in phase II and phase III trials, even if they are not the primary outcome measures. Scales such as the Barthel Index, Rankin scale, and Stroke Impact Scale reflect these other levels of functioning. Experience gained in the use of these scales in early clinical development and the data obtained can then be used to estimate with greater precision sample sizes that may have a greater possibility to detect a drug effect in a phase III trial. In vivo evaluation of ischemic damage or activity can be assessed by determining the extent of cytotoxic edema by diffusion-weighted MRI (DW-MRI) in relation to extent of hypoperfusion on perfusion-weighted MRI (PW-MRI) or by infarct size on delayed CT scan. The comparison of DW and PW-MRI images may help select patients who might benefit from treatment by providing a radiographic index of the extent of the penumbra, currently inferred on the basis of the amount of time elapsed from the onset of stroke symptoms. Additionally, the evolution of ischemic lesion volume on DW-MRI at baseline to T2 lesion volume on days 30 to 90 may provide a measure of drug effect in a relatively small sample size. The change in infarct size from a baseline CT or MRI is likely to be a better approach than measuring the final differences in infarct volume between treatment groups. Obtaining a baseline CT volume early after an acute stroke is difficult and thus limits this approach; DW-MRI overcomes this limitation and thus offers the possibility of determining a change in volume from baseline. Other markers of ischemic injury may be plasma levels of various substances released into the circulation from injured brain, including neuron-specific enolase, S-100, and thrombomodulin, among others. For thrombolytic therapy or mechanical recanalization, ultrasound or angiographic findings with contrast angiography, MR angiography, or CT angiography may provide surrogate markers of in vivo drug activity by measuring rates of recanalization.

The following examples are provided to illustrate certain particular features and/or embodiments. These examples should not be construed to limit the invention to the particular features or embodiments described.

### EXAMPLES

### Example 1

This Example describes materials and methods used to perform the subsequent Examples.

Mice. C57BL/6 mice (male, 8-12 weeks) were purchased from Jackson Laboratories. IRF7−/− mice were provided by Dr. Ian Rifkin (Boston University School of Medicine, Boston, Mass.). IRF3−/− mice were obtained from RIKEN BioResource Center. Both knock-out strains were back-crossed at least eight generations onto C57BL/6. All mice were housed in an American Association for Laboratory Animal Care-approved facility. Procedures were conducted according to Oregon Health & Science University, Institutional Animal Care and Use Committee, and National Institutes of Health guidelines.

TLR ligands. TLR4 ligand LPS (Escherichia coli serotype 0111:B4; Cat #L2630, purified by phenol extraction, protein content_3%) was obtained from Sigma-Aldrich. The TLR9 ligand CpG oligodeoxynucleotide (ODN) 1826 (tccatgacgttcctgacgtt), a mouse-specific phosphothioate unmethylated CpG ODN ligand for TLR9, was obtained from Invivo-Gen. InvivoGen has confirmed the specificity of ODN 1826 for mouse TLR9 by testing against cells transfected with the other TLR family members. In addition, endotoxin levels were determined to be negligible (<0.125 EU/mg). Systemic administration of TLR ligands was via either intraperitoneal injection or subcutaneous injection as noted. The TLR ligands were titrated for the optimal neuroprotective dose in the particular strain of mouse being tested to control for variation between product lots.

Middle Cerebral Artery Occlusion.

Mice were anesthetized and subjected to middle cerebral artery occlusion (MCAO) using the monofilament suture method. Briefly, a silicone-coated 7-0 monofilament nylon surgical suture was threaded through the external carotid artery to the internal carotid artery to block the middle cerebral artery; it was maintained intraluminally for either 12 min for ischemic preconditioning or 45 min for injurious ischemia. The suture was then removed to restore blood flow. Mice undergoing ischemic preconditioning followed by injurious ischemia were rested in their home cage for 72 h before receiving the second MCAO (45 min). Cerebral blood flow (CBF) was monitored throughout surgery by laser Doppler flowmetry. The mean CBF value between groups was consistent for each experiment. In addition, any mouse that did not maintain a CBF during occlusion of <20% of baseline was excluded from study. Body temperature was monitored and maintained at 37° C. with a thermostat-controlled heating pad.

Infarct Evaluation

Mouse brain was sliced into 7×1 mm coronal sections before 2,3,5-triphenyltetrazolium chloride TTC staining, and the volume of infarct was determined by summing of the area of infarct from individual slices. The infarct size for each image was determined using NIH image analyses. To account for edema within the infarct region, infarct area was computed indirectly as follows: 100×(contralateral hemisphere area−area of live tissue on ipsilateral hemisphere)/(contralateral hemisphere area).

RNA Isolation

Total RNA was isolated using the Qiagen RNeasy Lipid Mini Kit (Qiagen), or the RNAeasy Mini Kit (Qiagen).

Quantitative Real-Time PCR

Total RNA isolated from brain cortex, or RNA from cell culture, was reverse transcribed using an Omniscript Reverse Transcription kit (Qiagen). Quantitative PCR was performed using the TaqMan Gene Expression Assays (Applied Biosystems) on an ABI-prism 7700. Results were normalized to β-actin expression and analyzed relative to appropriate controls. The relative quantification of the gene of interest was determined using the comparative CT method (2^{-ΔΔCt})

Plasma Cytokine Evaluation

Blood was collected via cardiac puncture under isoflurane anesthesia 3 hours following subcutaneous administration of vehicle, IFN-b, DMXAA, compounds of the NA series, or compounds of the ND series. Plasma cytokine levels were evaluated using a cytokine 20-plex mouse ProcartaPlex kit (Invitrogen) for labeling of multiple cytokines within an individual sample and run on a Luminex 200 instrument.

Oxygen Glucose Deprivation In Vitro

Primary mouse mixed cortical cultures were prepared from embryonic day 15 to 17 mouse fetuses. Cortices were dissected and dissociated with trypsin-EDTA (Invitrogen) and plated at a density of 4.5×10⁵ cells/ml onto coverslips coated with poly-1-ornithine (15 mg/L). Cells were cultured in Neurobasal media (containing 4.5 g/L glucose; supplemented with Glutamax and B27-AO; Invitrogen) for 5 d before each experiment. Cultures consisted of ˜60% neurons (range, 53-66%) as determined by staining for neuronal-specific nuclear protein (Millipore Bioscience Research Reagents), with <5% astrocytes (GFAP⁺; Sigma) and <5% microglia (tomato lectin⁺; Vector Laboratories). Oxygen glucose deprivation (OGD) was performed by removal of the culture medium and replacement with Dulbecco's PBS (Invitrogen), followed by incubation in an anaerobic atmosphere of 85% N₂, 10% CO₂, and 5% H₂ at 37° C. for 3 h. The anaerobic conditions within the chamber were monitored using an electronic oxygen/hydrogen analyzer (Coy Laboratories). OGD was terminated by replacement of the exposure medium with Neurobasal medium (containing 4.5 g/L glucose; supplemented with Glutamax and B27-AO) and return of the cells to a normoxic incubator. Control plates were kept in the normoxic incubator during the OGD interval.

Pilot Screen

After reagent utilization and assay signal-to-noise optimization, a pilot screen was performed in triplicate on a 2560 compound library (The Spectrum Collection, MicroSource Discovery Systems, Inc.) containing therapeutics of known activity and natural products to develop and evaluate assay performance in our primary screen, counterscreen, and cross-species evaluation methods.

Primary Screen

THP1 Dual (InvivoGen) cell suspensions were cultured in RPMI 1640 (Hyclone, Logan, Utah) supplemented with 10% fetal bovine serum (Hyclone), 100 U/mL Normocin (Invivogen), and 50 U/mL PenStrep (Gibco, Dublin, Ireland) between a density of 7·10⁵-2·10⁶ cells/mL. Every other passage, media was supplemented with 5 U/mL Blasticidin and 100 U/mL Zeocin to maintain the expression constructs. White, flat-bottom 384-well plates (Greiner, Kremsmunster, Austria, 781080) were seeded with 2.5·10⁴ cells/well in a total of 50 μL media containing 5 ng/mL phorbolmyristic acid (PMA) using a Matrix WellMate (Thermo Scientific) dispenser. After 48 hours of incubation at 37° C., media was removed using a BioTek ELx405 CW plate washer set to aspirate and wash each well three times with 65 μL 1×PBS. Plates were reloaded with 50 μL media without PMA and treatments added. Lipopolysaccharide in dimethylsulfoxide (DMSO) was transferred to control wells using a HP D300 microdispenser to a final concentration of 313 ng/mL. Compounds from chemical library plates were copied using a Sciclone (Caliper LifeSciences) mounted with a 384-pin transfer tool to obtain a final concentration of 10 μM. After incubation with treatments for 24 hours, 10 μL of supernatant media from each well was transferred into a white, flat-bottom 384-well plate for luminescence readout and a black-walled, clear flat-bottom 384-well plate for absorbance readout.

### Example 2

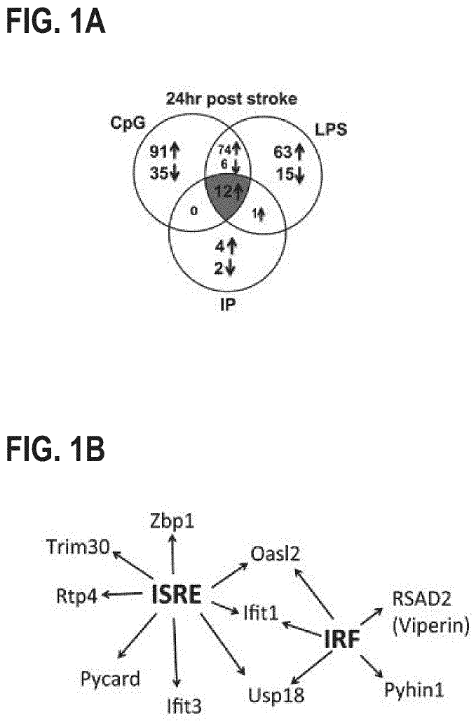

This example demonstrates the results of target identification through large-scale transcriptional analysis of several preconditioning paradigms. Briefly, preconditioning the brain prior to an ischemic event provides robust protection in several animal models of stroke, often resulting in a 50-70% reduction in ischemic volume. Three preconditioning paradigms, including two Toll-like receptor agonists, lipopolysaccharide (LPS) (corresponding to TLR4) and cytosine-phosphate-guanine (CpG) (corresponding to TLR9), and brief ischemia (IP) each resulted in a reprogrammed response to injury in the brain that was not evident in non-preconditioned mice. Following stroke, animals treated with any one of the above-mentioned three preconditioning stimuli tested shared a genomic fingerprint comprised of 12 genes (FIG. 1A). Specifically, FIG. 1A shows a Venn diagram comparing the reprogrammed genes in each of the preconditioning paradigms at 24 h post stroke. Promoter region analysis of these differentially regulated genes revealed an over-representation of interferon regulatory factor (IRF) sequence elements indicating that the ischemic response in preconditioned mice involves IRF-mediated transcription (FIG. 1B). Specifically, FIG. 1B shows a hypothetical gene-TRE network of genes (in grey) common to all conditions showing the relationship of identified transcriptional regulatory elements (TREs, in bold) to the regulated genes.

### Example 3

This example demonstrates that IRF3 activity plays a role in ischemic preconditioning, and that in an absence of IRF3 activity gene induction for a number of genes is impacted.

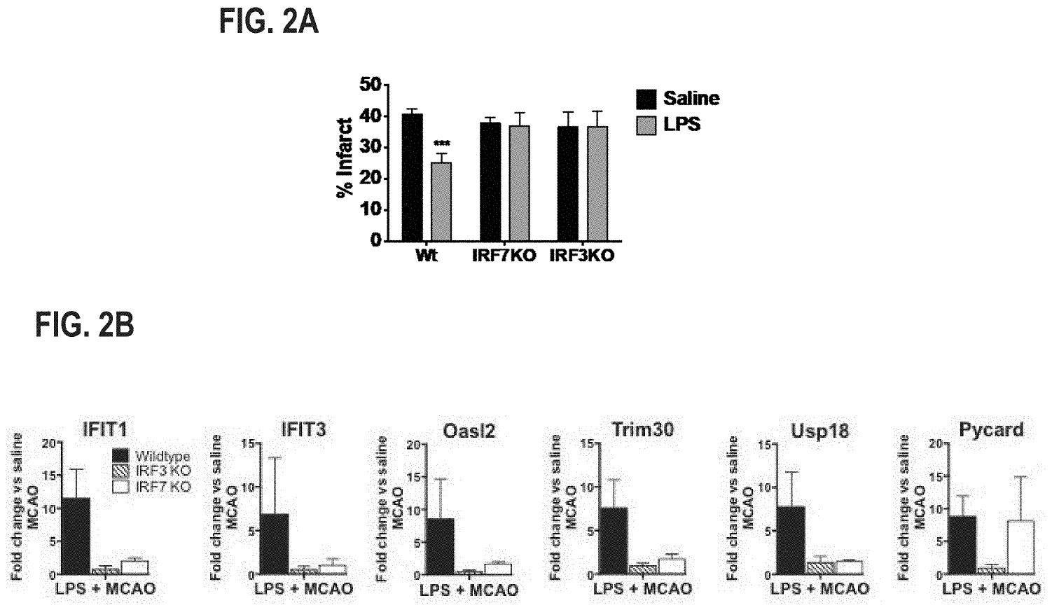

IRFs are master regulators of innate immune receptors, constituting a family of DNA-binding proteins expressed in a variety of tissues. Specific IRFs are known to bind to interferon-stimulated response elements (ISREs). To determine the role of ISRE-binding IRFs in neuroprotection, mice deficient in genes encoding IRF3 or IRF7 were preconditioned with the TLR4 agonist LPS three days prior to transient middle cerebral artery occlusion (MCAO). The potent preconditioning effect of LPS was completely abbrogated in IRF3 or IRF7 knockout (KO) mice, demonstrating a role for these regulatory factors in mediating protection from damage induced by MCAO (FIG. 2A). Specifically, IRF3 deficient and IRF7 deficient mice were preconditioned with LPS or saline 72 hours prior to MCAO. Infarct volume was measured 24 hours after surgery. Error bars represent means+/−SEM, ***p<0.001. In addition, brain gene transcription profiles for a subset of 6 of the 12 commonly induced genes (refer to FIGS. 1A-1B) were determined in the IRF3 and IRF7 KO mice in the context of stroke. In the genes tested, all but one failed to show induction post stroke in the IRF3 and IRF7 deficient mice (FIG. 2B). Specifically, gene transcription was measured in the brain 24 hours post MCAO in LPS preconditioned wildtype, IRF3 deficient, and IRF7 deficient mice using quantitative PCR. Data at FIG. 2B is represented as fold change vs genotype matched saline+MCAO (+/−SD; n=3-4 mice/genotype).

Data was acquired for preconditioning using CpG (TLR9 agonist). FIG. 2C shows results from an experiment where wild-type (n=8-13) or IRF7 KO (n=8-14) mice were pretreated with LPS (1.0 mg/kg), CpG (1.6 mg/kg), or saline 72 hours before 45 minutes MCAO. FIG. 2D shows results from an experiment where wild-type (n=8-10) or IRF3 KO (n=7-8) mice were pretreated with CpG (1.6 mg/kg) or saline 72 hours before 45 minutes MCAO. For both FIG. 2C and FIG. 2D, infarct volumes were measured 24 hours following surgery using TTC staining. Data at FIGS. 2C-2D are shown as group means+SEM; two-way ANOVA, Bonferroni post hoc, ***p<0.001 compared with saline-treated mice for respective genotype.

### Example 4

This example demonstrates a role for IFIT1 in ischemic preconditioning efficacy.

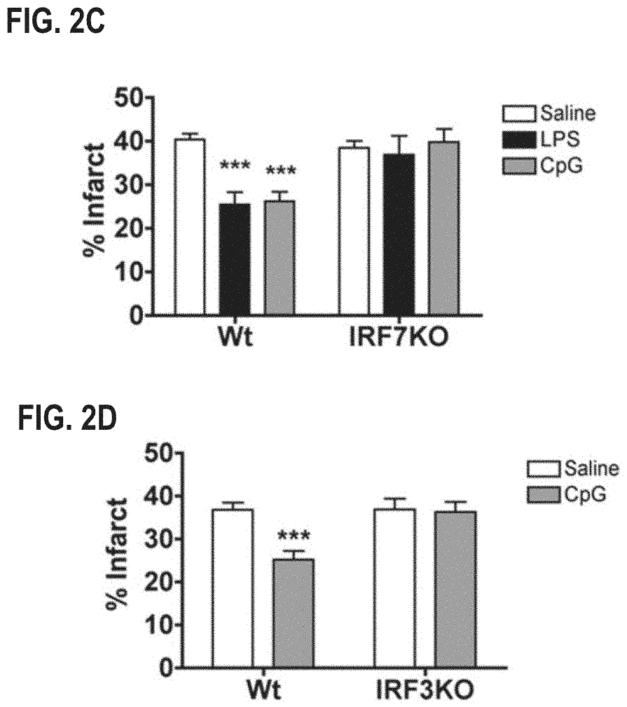

While traditional genomic analysis considers the behavior of each gene independently from all other genes, a complementary approach treats multi-stimulus and kinetic transcriptomics data as a co-expression network and then uses the topology of the network to identify points of constriction, or bottlenecks. Bottlenecks are predicted to represent points of control for transitions between system states that are important to the underlying biological conditions being studied. These theoretical methods can identify additional players in important biological processes, such as those involved in preconditioning-induced neuroprotection. The identification and validation of functional bottlenecks predicted by network analysis was predicted to provide insight into the dynamics of preconditioning and its regulation in the brain, and could thus serve as a means to identify elusive drug targets. Using this method on the transcriptional data set described above, IFIT1 was identified as a predicted bottleneck gene. To determine the biological relevance of this prediction, the role of IFIT1 in LPS preconditioning against ischemic injury in mice was examined. IFIT1-deficient mice were preconditioned with LPS three days prior to MCAO and neuroprotection was completely abrogated in the IFIT1-deficient mice (FIG. 3), indicating that IFIT1 is required for LPS preconditioning-induced protection. Specifically, at FIG. 3, IFIT1 KO mice and WT mice preconditioned with LPS or saline 72 hours prior to MCAO are shown. Infarct volume was measured 24 hours after surgery. Group mean+/−SEM are shown; ** p<0.01.

### Example 5

This example demonstrates that poly ICLC has a protective effect in terms of cell death in the context of stroke.

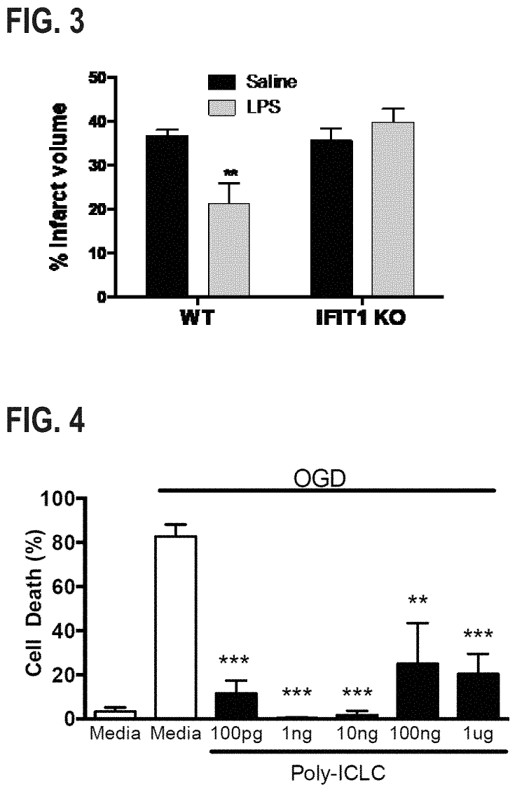

To validate that acute protection of IRF activation could protect brain cells from ischemia the effect of poly ICLC (PIC) on primary mixed cortical cultures from mouse brain in the context of oxygen glucose deprivation (OGD), mimicking the effects of stroke, was examined. PIC is an innate immune activator that acts primarily by inducing an anti-viral gene signature including induction of IRF-associated pathways. Treatment of cortical neurons with PIC after exposure to OGD resulted in significant protection when cell death was examined 24 hours later (FIG. 4). Specifically, at FIG. 4, primary cortical cultures were exposed to OGD after which poly ICLC was added. Cell death was assessed 24 hours later. **p<0.01, ***p<0.001. Data reflect mean+/−SEM. The data at FIG. 4 suggests that stimulation of IRF activity could provide protection in the brain in vivo. It has been found that subcutaneous injection with PIC elicits brain expression of IRF transcribed genes, IFIT1 and Usp18 (Table. 9), however this potent innate immune stimulator also elicits unrelated pathways (e.g., TNF) that could enhance immunotoxicity or have untoward effects when given after stroke in humans. Therefore, it was desired to identify IRF activators with minimal pro-inflammatory effects. Specifically, Table 9 depicts fold-change vs vehicle as post-SC injection of PIC (40 μg) or 0.5% CMC vehicle, values normalized to beta-actin. Fold change vs vehicle determined using comparative CT method (2\hat{ }−ddCT). ND=not determined.

TABLE 9
PIC induced IRF activity in Mouse Brain

| | Fold change vs. vehicle* | | |
|---|---|---|---|
| Genes | 3 hrs | 6 hrs | 72 hrs |
| Ifit1 | 30.76 ± 4.0 | 36.71 ± 8.9 | 3.13 ± 1.2 |
| Usp18 | 30.54 ± 4.0 | 41.92 ± 13.8 | 1.91 ± 1.5 |
| ISG15 | ND | ND | 3.29 ± 1.0 |

*post-SC injection of PIC (40 ug) or 0.5% CMC vehicle, values normalized to beta-actin. Fold chg vs vehicle using comparative CT method (2^{∧}-ddCT).
ND = not determined.

### Example 6

This example demonstrates that DMXAA and 5′ppp dsRNA are potent activators of ISRE in mouse macrophage cells.

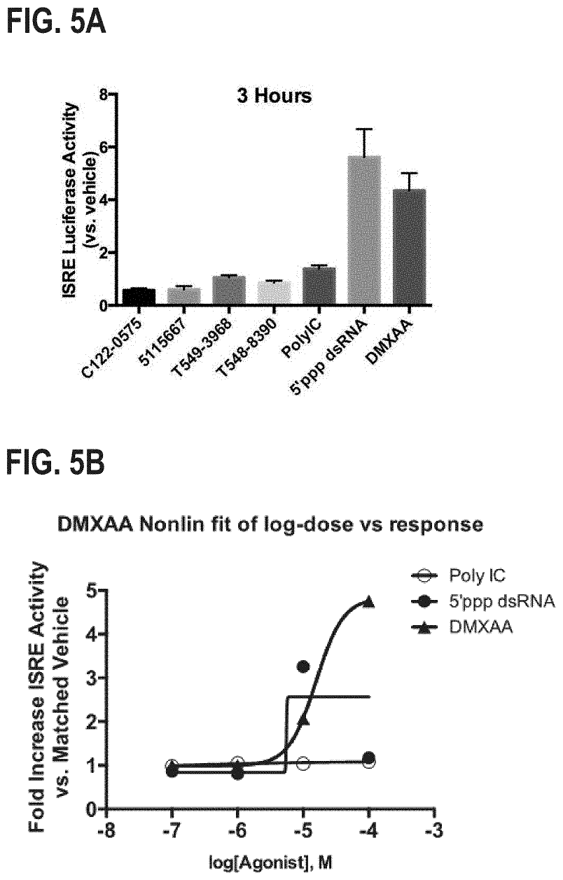

To characterize a small directed subset of molecules known to activate IRF3, mouse macrophage cells (RAW264.7) containing an ISRE-firefly luciferase reporter with a constitutively active renilla luciferase internal control for normalization (Promega) was utilized. IRF3 can activate ISRE through direct binding and via feed-forward mechanisms through potent induction of IFNβ. Using a 96-well plate format, 5′ppp dsRNA and DMXAA were identified as the most potent activators of ISRE at 3 hours post-treatment (FIG. 5A). Specifically, RAW264.7 cells containing ISRE firefly luciferase reporter were treated with indicated compounds for 3 hours and luciferase activity was determined. DMXAA was the only compound that induced a robust dose-dependent and prolonged activation of ISRE lasting 20 hours post-treatment (18.6+/−3.63 fold increase versus vehicle) (FIG. 5B). Specifically, an expanded dose-response study shows DMXAA has superior drug-like properties for activating mouse ISRE (EC50 ˜16 μM). DMXAA and 5′ppp dsRNA were selected for further analysis of genomic responses in central nervous system (CNS) cells.

### Example 7

This example demonstrates that DMXAA induces IRF3-related genes much more robustly than inflammatory genes.

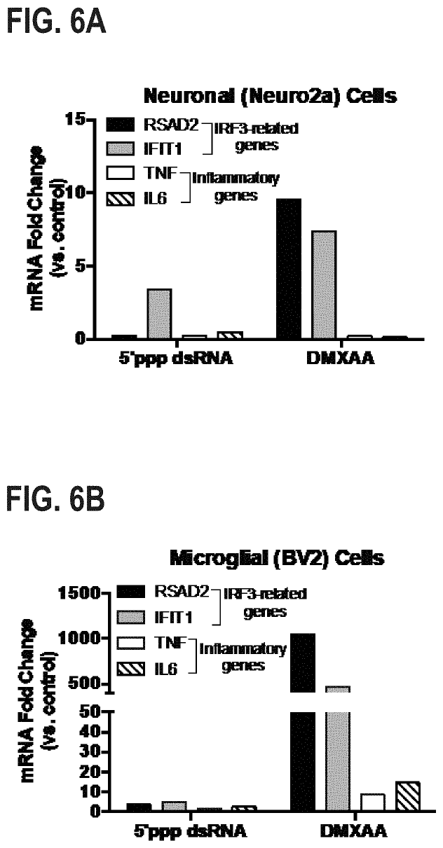

The response of a mouse neuronal cell line (Neuro2a) and microglial cell line (BV2) to DMXAA and 5′ppp dsRNA were examined. DMXAA stimulated the most robust induction of IRF-regulated genes RSAD2 and IFIT1, compared to 5′pppRNA (FIGS. 6A-6B). Since inflammatory pathways mediated by NF-κB are known to have negative effects on stroke outcome, the induction of key detrimental genes including TNF and IL-6 in response to drug treatment was evaluated. The ratio of IRF to TNF activity was 10:1 and 100:1 in Neuro2A and BV2 cells treated with DMXAA, respectively (FIGS. 6A-6B). Thus, the IRF-mediated genomic response was much more robust that its induction of inflammatory genes, indicating that IRF signalling predominates in response to DMXAA treatment. Specifically, Neuro2A (FIG. 6A) or BV2 (FIG. 6B) cells were treated with 5′ppp dsRNA or DMXAA (10 ug/ml) for 3 hours. RNA was collected from cell pellets and qtPCR was performed with indicated primers. Values are normalized to actin and are presented as fold change over vehicle control.

### Example 8

This example demonstrates DMXAA induces a number of IRF3 related gene expression, is protective against stroke, and that IRF3 is involved in the DMXAA-based protective effect.

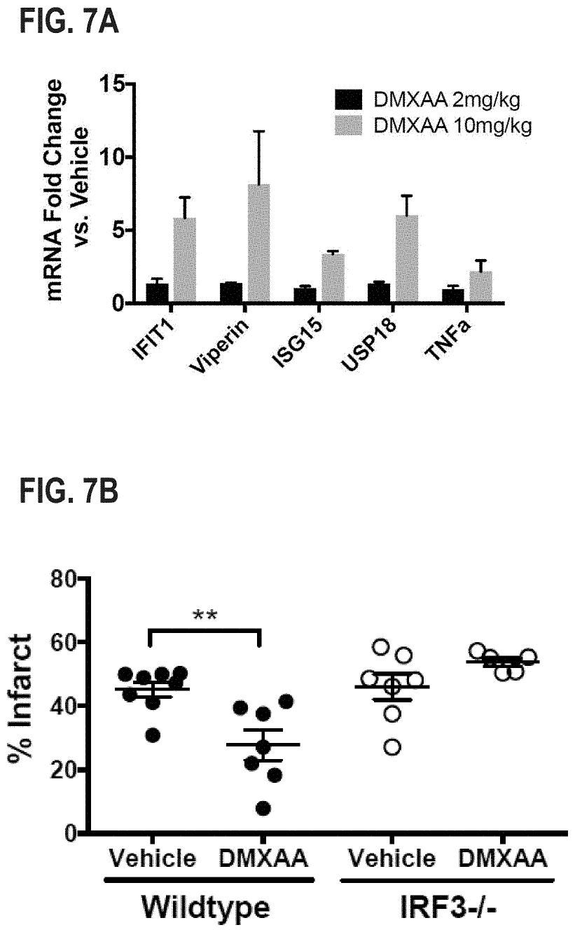

To determine if DMXAA could elicit detectable IRF activation in vivo via a therapeutically relevant route, mice were administered DMXAA intraperitoneally (ip) and brain tissue was collected at 3 hours. DMXAA at 10 mg/kg resulted in a significant increase in IRF3 related gene expression with minimal induction of pro-inflammatory genes TNF, while a 5-fold lower dose resulted in minimal increase in IRF-mediated gene induction in the brain compared to vehicle (FIG. 7A). Specifically, with regard to FIG. 7A, male C57/BL6 mice were given DMXAA (2 mg/kg or 10 mg/kg, IP) and brain cortical tissue was collected at 3 hours. Values depicted are group means+/−SEM; n=4 per group.

To determine the effect of DMXAA on cerebral ischemic injury, male mice underwent 60 minute MCAO and were treated at the onset of reperfusion with either vehicle or 250 μg DMXAA (˜10 mg/kg, IP). Infarct volumes were measured at 24 hours. Wild-type mice treated with DMXAA showed a significant 2-fold reduction in ischemic injury compared to controls (FIG. 7B). Alternatively, mice lacking IRF3 were not protected by treatment with DMXAA (FIG. 7B). Shown for each group are individual data points+/−group SEM, n=7-8 per group, ** p<0.01.

### Summary of Examples 2-8

Taken together, the data shown at Examples 2-8 support that IRFs and one or more downstream IRF-inducible genes represents an endogenous path to neuroprotection that can be commandeered in vivo to provide acute protection against stroke. The compounds discussed herein (e.g., NA chemofamily and ND chemofamily) comprise IRF activators with minimal off-target immune activity and specificity for both mouse and human for use in stroke therapy.

### Example 9

This example demonstrates viability of a high throughput approach for identification of potent and selective IRF activators as herein disclosed.

Optimal assay conditions were determined in terms of cell titration, reagent selection, use of differentiated or undifferentiated THP1 cells, extent and sources of variability, and assay reproducibility, for a high-throughput primary screening assay to identify potent and selective IRF activators using a human THP1 dual ISRE/NF-κB reporter cell line and 384-well format. Assay quality and Z-prime (high signal-to-background ratio) was confirmed by evaluating the dose response and cytotoxicity of positive control agents and a mini-screen set of known or predicted-to-be activators of ISRE and NF-κB. The 50% cytotoxic and effective doses (CC50, EC50, respectively) and ISRE:NF-κB ratios were determined.

Specifically, to enable screening of novel chemical entities (NCEs) in a larger screening effort, a robust high-throughput cell-based assay system for primary hit identification of ISRE activators based on the above-mentioned target validation was desired. Human ISRE activity was verified using a THP-1 monocyte line containing the NF-κB-SEAP and IRF-Lucia luciferase dual reporter (THP1-Dual; Invivogen) that features two reporter genes, SEAP (secreted embryonic alkaline phosphatase) and Lucia luciferase, thereby enabling the simultaneous study of the NF-κB and IRF signalling pathways. The Lucia luciferase reporter is under the control of an ISG54 (interferon-stimulated gene) minimal promoter in conjunction with five IFN-stimulated response elements. The SEAP gene is driven by an IFN-β minimal promoter fused to five copies of the NF-κB consensus transcriptional response element and three copies of the c-Rel binding site. Both reporter proteins are readily measurable in the cell culture supernatant when using QUANTI-LUC™ and QUANTI-Blue SEAP detection reagents.

The inclusion of NF-κB as a counter screen in primary hit identification is important for the following reasons: 1) NF-κB is suppressed following stroke in preconditioned animals suggesting that activation of this pathway after stroke may be counter-productive, 2) pharmacological inhibition of NF-κB causes a reduction in stroke volume in rodents, and 3) the use of TNF inhibitors in the brain is an effective stroke therapeutic strategy due to the damaging role of the NF-κB-inducible cytokine. Thus, from a drug-development perspective, identification of molecules that activate IRF without effects on NF-κB was predicted to be an important strategy early in the hit selection process. Since NF-κB activity appears unnecessary for acute experimental stroke efficacy, it was thus desired to avoid this activity when selecting candidate molecules.

Experimental Design

Using known IRF activators (e.g., purified IFN, 5′ppp-dsDNA, PIC, and other commercially available agents including idarubicin), a known NF-κB activator (e.g., purified human TNF), and a dual IRF/NF-κB activator (e.g., LPS), appropriate assay conditions for both reporters in a miniaturized 384-well assay format were determined. Optimal cell density, reagent selection, assay duration, and need for use of differentiated or undifferentiated THP1 cells was assessed. Reproducibility of the optimized assay was also systematically assessed by performing multiple test runs and calculating coefficient of variation.

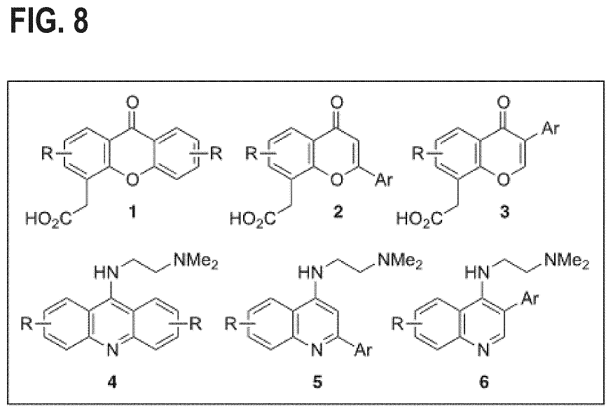

After determining the appropriate assay conditions, a screening was conducted of a small library augmented with compounds that are structurally similar to DMXAA (1), FAA (2 and 3), and L56 (5 and 6) (see FIG. 8, where R is one or more aromatic ring substituents and Ar is an aromatic or heteroaromatic ring). Acridines (4) were also included because they represent a combination of the structural characteristics of DMXAA and L56. These compounds were obtained from existing compound libraries in the Experimental Chemotherapy Lab at the Portland VA Medical Center in Oregon, commercial sources, or chemical synthesis. Primary screening included a single data point or dose for each molecule and a confirmatory assay was performed using an 8-point dose-response mode. Primary and secondary screens were repeated multiple times using the focused library in the dual reporter assay and coefficient of variation calculated among assay iterations. After normalization was applied for each assay run, “hit” selection was performed on the plates that passed quality control (QC) criterion. Hits were defined as test samples having values above or below the activity cutoff determined based on noise level of the assay to reduce false positive rates. With hits from the primary screen, the EC50 and CC50 was determined under the optimal assay conditions, and the ratio of ISRE:NF-κB activity as well as the extent of cytotoxicity was determined in the same assay, by measuring viability the same day biological activity measures were taken.

Data Analysis and Statistics

GraphPad Prism and/or Excel software was used for data analysis and statistics where possible. To address “random” assay variability (unexplained variance in raw data across plates) normalization of data within each plate was performed by calculating percent of control (PC) defined as the activity of the i^{th} sample (Sᵢ) divided by the mean of either the positive or negative control wells (C). This was necessary to enable comparison of results across plates or experiments and to allow a single cut-off for hit selection. A controls-based approach to normalization was used provided that positive controls performed adequately. In the controls-based approach, the assay-specific in-plate positive and negative controls were used as the upper (100%) and lower (0%) bounds of the assay activity, and the activities of the test samples were calculated with respect to these values. Using non-controls based approaches would be less appropriate given that a focused library for initial screening was used, and thus it cannot be assumed most will be inactive. Plate-wise versus experiment-wise normalization was examined and the method that fits best with experimental results was selected. To determine if the data collected from each plate meet the minimum QC requirements, and if any patterns exist before and after data normalization, the distribution of control and test sample data was examined at the experiment-, plate- and well-level. Using Z′ or Z-factor quality control assessment may not always be appropriate with a focused library and in such cases signal window or signal to noise approaches were used. Additionally, outliers may cause distortions to the normal distribution of the data and impact results negatively, thus our HT data set may be analyzed using the robust statistics method. In some examples, the median and median absolute deviation (MAD) was utilized as statistical parameters as opposed to mean and standard deviation (SD), respectively, to diminish the effect of outliers on the final analysis results.

Optimized Assay Conditions

Using the experimental design and data analysis outlined above, optimized assay conditions were obtained. The responsiveness of the human THP-1 Dual cell line was evaluated in an array of assay conditions to obtain sensitive and reproducible results for the activation of IRF and NF-κB reporters. An optimal plating density and differentiation protocol for these cells was established in a 384-well format. All transfer steps are fully automated using high throughput robotic equipment. Briefly, cells are seeded in differentiation medium using a Matrix WellMate fitted with the WellMate Stacker (Thermo Scientific) and incubated at 37° C. with 5% CO₂ for 48 h. Following differentiation, cells are washed using a BioTek ELx405 Select CW plate washer and growth medium added with the Matrix WellMate. Programmed control treatments are added to wells using a D300 digital dispenser fitted with T8+ dispense heads (Hewlett Packard) designed to dispense nanoliter quantities of material. Compounds from library plates are added using a pin tool fitted to a Sciclone ALH 3000 Workstation (Caliper Life Sciences) programmed with Maestro v4.4. After treatment, plates are returned to incubators for 24 hours. To measure reporter expression, supernatant is collected using the Sciclone and dispensed into 2 plates: 1) a white 384-well assay plate for Lucia luminescence measurements and 2) a clear-bottom black 384-well assay plate for SEAP absorbance measurements. For Lucia luminescence plates, the detection reagent QUANTI-Luc (InvivoGen) is added with the bulk dispenser and luminescence measured within 5 minutes on a BioTek Synergy4 machine. To the SEAP plates, the detection reagent QUANTI-Blue (InvivoGen) is added with the bulk dispenser, followed by a 1 h incubation at 37° C. Edge effects in the SEAP assay are eliminated using plate-sealing film during incubation. The film is removed and plates are then read at 630 nm on the BioTek Synergy4.

Confirmation of Assay Quality and Z-Prime (High Signal to Background Ratio with Low Signal Variance) by Evaluating the Dose-Response of Positive Control Agents and a Mini-Screen Set of Known or Predicted to be Activators of ISRE and/or NF-κB and Define the 50% Cytotoxic and Effective Doses (CC50, EC50) and ISRE:NF-κB Ratio

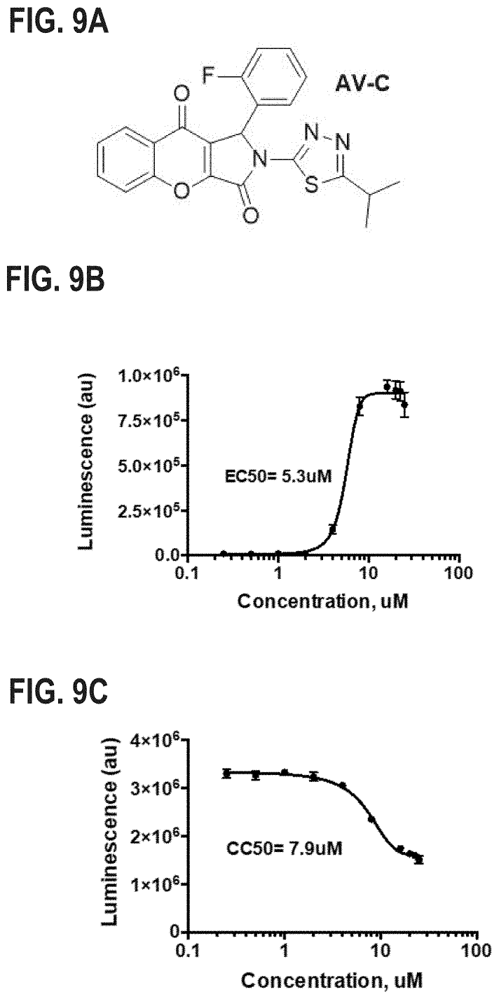

Endotoxin (lipopolysaccharide, LPS) was used as the high-signal positive control for activation of both IRF and NF-κB. AV-C (FIG. 9A) is a human specific IRF activator used as an intermediate-signal positive control (˜10% of LPS signal intensity) for the human THP1-Dual cell assays. AV-C shows only minimal IRF activation in mouse cells (see below J774-Dual cell activation), and was thus considered as a control compound for the THP1-Dual screening assay. An 8-point dose response of AV-C was performed and an EC50 (50% effective concentration) of 5.3 μM for IRF activation (FIG. 9B and Table 10) was obtained. Furthermore, a CC50 (50% cytotoxic concentration) of 7.9 μM (FIG. 9C and Table 2) was obtained. The results indicate a toxicity index (Table 10) of 1.5 for AV-C, which represents a narrow window for activity versus toxicity. In the high throughput screen to identify novel IRF modulatory agents (discussed in further detail below), hits identified in the primary screen undergo secondary screening, as demonstrated here for AV-C, to determine EC50 and CC50 values using an 8-point dose response curve. The toxicity index for these measurements is then calculated and used for ranking of compound hits.

TABLE 10
THP1-Dual control compound responses.

| | EC₅₀ ISRE | | Selectivity Index |
|---|---|---|---|
| | activation | CC₅₀ | (CC50/EC50) |
| LPS (ng/mL) | 29.8 | >5000 | >150 |
| AV-C (μM) | 5.3 | 7.9 | 1.5 |

### Example 10

This example demonstrates the identification of IRF selective compounds via high throughput screening methodology as disclosed herein.

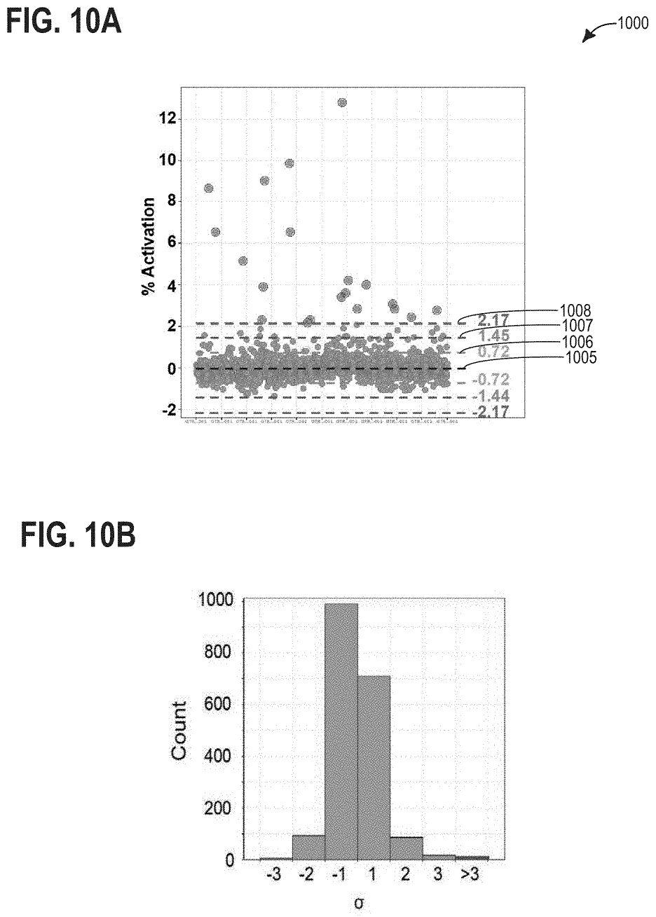

A THP1-Dual pilot library screen of over 1900 compounds from the Spectrum Collection library (MicroSource Discovery Systems, Inc) was performed to evaluate the performance of the optimized screen in the context of a high-throughput protocol. The library compounds spanned 6 plates and LPS and AV-C were used as positive controls on each plate for normalization between plates. LPS activation of each reporter (IRF-Lucia and NF-κB-SEAP) was set at 100% activation, with test compounds being reported as % of LPS activation. Three independent iterations of the screen were performed to evaluate reproducibility and reliability of the output. Assay conditions were performed as described above in Example 9, with compounds (10 μM) incubated with cells for 24 hours prior to screening for secreted reporters, Lucia and SEAP. Both the IRF-Lucia and NF-κB-SEAP produced high Z-prime values (Table 11) indicating robust assay conditions across the replicate rounds of screening. For the pilot screen, IRF specific hits were defined as compounds that elicited percent activation >3σ above the mean for ISRE activation (FIGS. 10A-10B), and <16 for NF-κB activation for all library samples on a per plate basis, with the added selection of an ISRE/NF-κB ratio of ≥2. Specifically, illustration 1000 at FIG. 10A depicts percent activation relative to the LPS control for each sample ID number for an assay for IRSE activation. The mean of the samples 1005 is depicted, as well as +1 (1006), +2 (1007) and +3 (1008) standard deviations (a) from the mean (1005). Also shown but not labeled are corresponding −1, −2 and −3 standard deviations. Samples with activation above 3a (three standard deviations) are labeled as hits. FIG. 10B shows the distribution of samples about the mean to illustrate that the output follows a normal distribution. An average IRF specific hit rate per round was found to be 5/1920, or 0.2%, suggesting that in the high throughput screen of ˜250,000 compounds discussed in further detail below, it may be expected to obtain ˜500 hits in the primary screen. Such a number represents a manageable number of compounds that advance to the secondary screening platform (8-point dose response) for verification of activity and determination of EC50 and CC50 values.

TABLE 12
Z-prime scores from Spectrum Library Screen in THP1-Dual cells.

| | Z-prime | Z-prime | |
|---|---|---|---|
| Assay | median | mean | Z-prime standard deviation |
| IRF-Lucia | 0.80 | 0.70 | 0.22 |
| NFkB-SEAP | 0.84 | 0.83 | 0.04 |

As an example of the type of compounds expected to see from the high throughput screen, compounds in the pilot screen that met the hit criteria of >3σ for ISRE activation and <16 for NF-κB activation on all 3 independent screening rounds were identified. A total of 6 compounds were identified with the average percent ISRE activation for these hits ranging from ˜2-6% that of the high-signal control, LPS (Table 13). These results are within the range of the control AV-C which gives a signal ˜10% of the LIPS control at its EC50. The compounds identified contained antineoplastics (Daunorubicin) and DNA intercalators (Quinacrine Hydrochloride), families of compounds that have been reported to induce IRF pathways. One of the compounds, Penfluridol, demonstrated significant IRF selectivity with an ISRE/NF-κB average ratio of 25.9 across the 3 screens. These results demonstrate the robustness of the human cell line assay and the ability to identify promising compounds for future drug development

TABLE 13
IRF hits identified in the human THP1 screen.

| | | Average | |
|---|---|---|---|
| Compound ID | Average ISRE | ISRE/NFκB | |
| (common name) | % Activation* | (Range) | Structure |
| OTR-0000145-001 (Penfluridol) | 5.5 +/− 1.1 | 25.9 (9.7-50.8) | 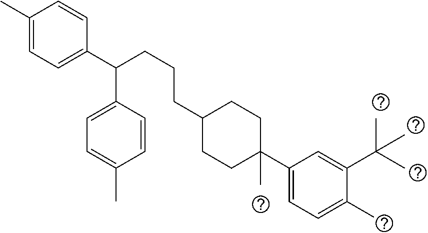 [SMILES: Cc1ccc(C(CCCC2CCC(C)(c3ccc(C)c(C(C)(C)C)c3)CC2)c2ccc(C)cc2)cc1] |
| OTR-0000510-001 (Quinacrine Hydrochloride) | 5.7 +/− 2.9 | 1.4 (1.0-2.0) | 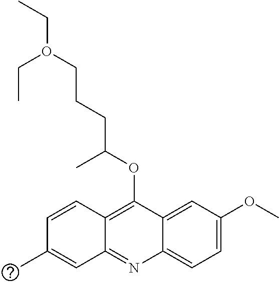 |
| OTR-0000708-001 (Methapyrilene Hydrochloride) | 3.5 +/− 2.6 | 1.4 (0.4-2.3) | 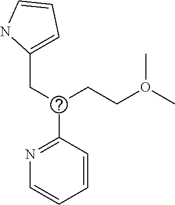 |
| OTR-0000353-001 (Daunorubicin) | 4.0 +/− 1.8 | 0.7 (0.2-1.0) | 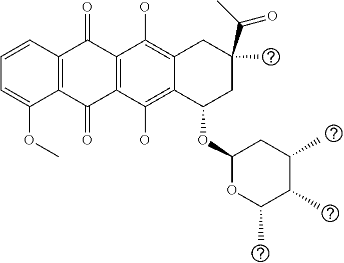 [SMILES: COc1cccc2c1C(=O)c1c(O)c3c(c(O)c1C2=O)C[C@@](C)(C(C)=O)C[C@@H]3O[C@H]1C[C@H](C)[C@H](C)[C@H](C)O1] |
| OTR-0001483-001 (Totarol-19-Carboxylic acid, methyl ester) | 2.0 +/− 1.0 | 0.4 (0.2-0.7) | 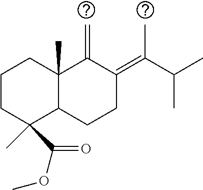 [SMILES: C=C1/C(=C(\C)C(C)C)CCC2[C@]1(C)CCC[C@]2(C)C(=O)OC] |
| OTR-0001624-001 (Obtusaquinone) | 1.7 +/− 0.6 | 0.2 (0.2-0.3) | 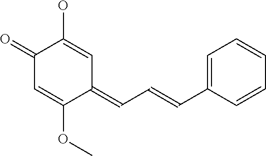 [SMILES: COC1=CC(=O)C(O)=C/C1=C\C=C\c1ccccc1] |

*Percent of LPS activation signal +/− SD.

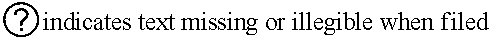

### Example 11

This example demonstrates cross-species activity of IRF specific compounds as identified via the high throughput screening methodology disclosed herein.

The pilot screening assay referred to above was conducted in mouse J774 cells containing dual ISRE/NF-κB reporters to evaluate cross-species activity of the hits retrieved by the screen discussed at Example 10.

First, optimal assay conditions and assay quality in 384-well format were determined. As mentioned, the mouse macrophage J774 cell line (J774-Dual, InvivoGen) stably transfected with the same inducible reporters described above (e.g., IRF-Lucia luciferase and NF-κB-SEAP) was used. The responsiveness of the J774-Dual cell line was investigated in an array of assay conditions to obtain sensitive and reproducible results for the activation of the IRF and NF-κB reporters. An optimal plating density and conditions for these cells was established for these cells in a 384-well format. Assay work-flow is similar to that discussed above for the THP1-Dual assay without a need for cell differentiation. Thus, cells are seeded directly in growth medium and incubated at 37° C. in a 5% C02 incubator for 24 hours prior to the programmed treatments being added as described herein.

Similar to the THP1-Dual assay, LPS was utilized as the high-signal internal control for both the ISRE and NF-κB readouts. Instead of the human-specific AV-C compound, the mouse-specific ISRE activator DMXAA was utilized. The 50% maximum response to DMXAA occurs at 24 μM with a 50% cytotoxic concentration >89 μM, representing a toxicity index of >3.7 (Table 14). Due to the differences in the mouse and human IRF activating pathways, DMXAA is not effective at activating ISRE in the THP1-Dual cell line. AV-C shows slight ISRE activity in the J774-Dual cell line, with a response that is 10% of LPS maximum compared to 75% in the human THP1-Dual cells at an equivalent dose (25 μM). It may be understood that use of the mouse J774-Dual cell line in addition to the human THP1-Dual cell line is to identify compounds with activity in both mouse and human cells in order to enable use of the cost-effective and robust mouse stroke model to test the efficacy of these candidate therapeutics for neuroprotection against brain ischemic injury, prior to more costly studies in primates or clinical trials

TABLE 14
J774-Dual control compound responses.

| | EC₅₀ ISRE | | Selectivity Index |
|---|---|---|---|
| | activation | CC₅₀ | (CC₅₀/EC₅₀₎ |
| LPS (ng/mL) | 370 | >5000 | >13.5 |
| DMXAA (μM) | 24 | >89 | >3.7 |

J774-Dual Pilot Library Screen

To assess the performance of the J774-Dual screening platform, the Spectrum Collection library mentioned above at Example 10, was used. Three independent rounds of the screen were performed to determine assay reproducibility. Similar to results obtained with the THP1-Dual cells, both ISRE-Lucia and NFκB-SEAP reporter readouts indicated superior quality performance with median Z-prime values of 0.73 and 0.93, respectively (Table 15). IRF selective hits were identified as above (ISRE>3σ, NF-κB<1σ, ISRE/NF-κB>2), and an average IRF specific hit rate in the J774 screen was found to be 14/1920, or 0.7%, slightly higher than in the human THP1 cells.

TABLE 15
Z-prime scores from Spectrum Library
Screen in J774-Dual cells.

| | Z-prime | Z-prime | Z-prime standard |
|---|---|---|---|
| Assay | median | mean | deviation |
| IRF-Lucia | 0.73 | 0.71 | 0.12 |
| NFκB-SEAP | 0.93 | 0.93 | 0.03 |

As an example of potential J774 IRF selective hits, compounds were identified that were >36 from the mean for ISRE activation in all three screening rounds (Table 16). Just one of the 4 compounds, Baicalein (Table 16) also met the criteria of low NF-κB activation (<1a from the mean; ISRE/NF-κB>2). Similar to the THP1 screen, antineoplastic (Daunorubicin, Doxorubicin) and DNA intercalation compounds (Doxorubicin) were also identified in the J774 screen, although with high NF-κB activity. Importantly, Daunorubicin induced ISRE activation in both the THP1-Dual and J774-Dual screens indicating that it has cross-species ISRE activity, demonstrating the feasibility of identifying/developing an ISRE activating compound with cross-species activity that could be tested in mouse models of stroke prior to translational development in higher order species.

TABLE 16
IRF hits identified in the mouse J774-Dual screen.

| | | Average | |
|---|---|---|---|
| Compound ID | Average ISRE | ISRE/NFκB | |
| (common name) | % Activation* | (Range) | Structure |
| OTR-0001580-001 (Baicalein) | 4.4 +/− 1.2 | 2.2 (1.2-2.5) | 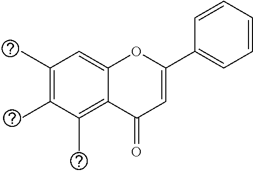 [SMILES: Cc1cc2oc(-c3ccccc3)cc(=O)c2c(C)c1C] |
| OTR-0000780-001 (Tacrolimus) | 28.3 +/− 13.0 | 0.3 (0.15-0.37) | 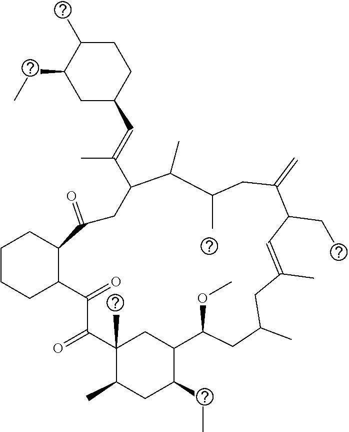 [SMILES: C=C1CC(C)C(C)C(/C(C)=C/[C@@H]2CCC(C)[C@H](CC)C2)CC(=O)[C@@H]2CCCCC2C(=O)C(=O)[C@]2(C)CC([C@@H](CC)C[C@H]2C)[C@@H](OC)CC(C)C/C(C)=C/C1CC] |
| OTR-0000353-001 (Daunorubicin) | 5.4 +/− 1.6 | 0.1 (0.09-0.18) | 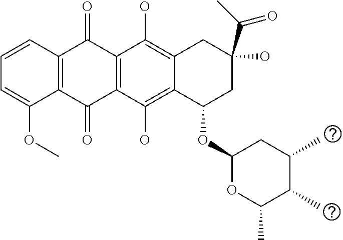 [SMILES: COc1cccc2c1C(=O)c1c(O)c3c(c(O)c1C2=O)C[C@@](O)(C(C)=O)C[C@@H]3O[C@H]1C[C@H](C)[C@H](C)[C@H](C)O1] |
| OTR-0000901-001 (Doxorubicin) | 5.2 +/− 1.5 | 0.2 (0.16-0.23) | 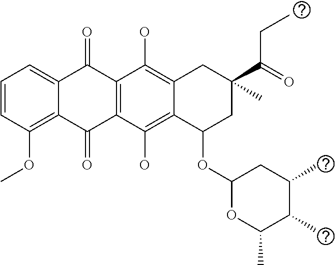 [SMILES: CCC(=O)[C@]1(C)Cc2c(O)c3c(c(O)c2C(OC2C[C@H](C)[C@H](C)[C@H](C)O2)C1)C(=O)c1c(OC)cccc1C3=O] |

*Percent of LPS activation signal +/− SD.

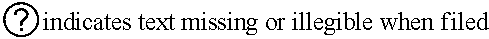

### Example 12

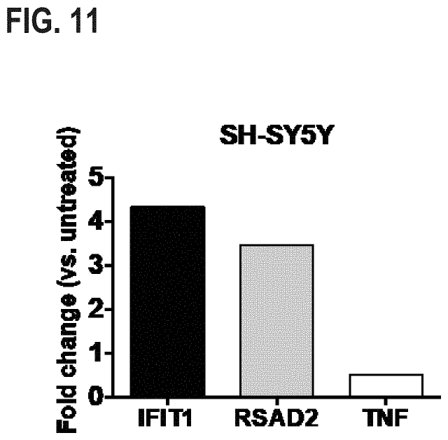

This example demonstrates AV-C induction of IRF-related genes. As discussed above with regard to Example 7, it was shown that DMXAA induced IRF-related genes RSAD2 and IFIT1 in both a mouse microglial and neuronal cell line with minimal induction of inflammatory genes TNF and IL6. To determine if the human specific IRF activator, AV-C could induce IRF activated genes, the human neuroblastoma cell line SH-SY5Y was stimulated with 20 μM AV-C for 3 hours. Preliminary results show that both IFIT1 and RSAD2 were increased while TNF showed no increase in response to AV-C (FIG. 11). Specifically, as mentioned, SH-SY5Y cells were treated with 20 μM AV-C for 3 hours. RNA was collected from cell pellets and qtPCR was performed with indicated primers. Values at FIG. 11 are normalized to actin and presented as fold change over vehicle control. The results indicate that IRF signaling pathways dominated the response to AV-C in this human central nervous system (CNS) cell line with minimal pro-inflammatory activation.

### Example 13

This example demonstrates identification of lead compounds for therapeutic development of an acute neuroprotectant using an experimentally validated high throughput screening platform with a goal of identifying compounds with acceptable target specification for preclinical profiling prior to in vivo efficacy testing.

The primary high throughput screen used the HTS platform of human THP1 monocyte cells containing the IRF-Lucia luciferase and NFκB-SEAP (secreted embryonic alkaline phosphatase) dual reporter (THP1-Dual, Invivogen), enabling the simultaneous study of the IRF and NFκB signaling pathways respectively. As discussed, the Lucia luciferase reporter gene is under the control of an ISG54 (interferon-stimulated gene) minimal promoter in conjunction with five interferon-stimulated response elements (ISRE). The SEAP gene is driven by an IFNβ minimal promoter fused to five copies of the NFκB consensus transcriptional response element and three copies of the c-Rel binding site. Both IRF-Lucia luciferase and NFκB-SEAP reporter proteins are secreted and readily measurable in the cell culture supernatant when using QUANTI-Luc™ and QUANTI-Blue™ detection reagents, respectively. Identified “hits” were confirmed in a secondary screen with an 8-point dose response curve. Hits were further screened for activity in the mouse using the mouse J774 macrophage-like cell line containing the identical reporter constructs (J774-Dual, Invivogen).

Experimental Design

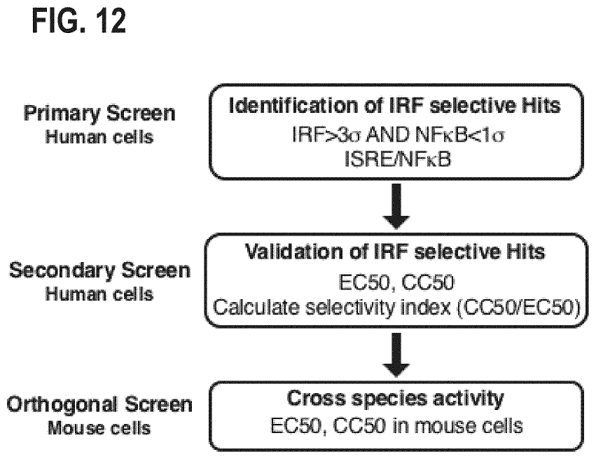

The OTRADI Small Molecule Library of approximately 246,000 diverse compounds (see below for description) was screened using the THP1-Dual based HTS platform described above. All transfer steps in the screen are fully automated, thus reducing variability. Primary screening included a single data point or dose for each molecule. After data normalization was applied for each assay run, “hit” selection was performed on the plates that pass the quality control (QC) criterion. Hits were defined as samples having values above the activity cutoff determined based on noise level of the assay to reduce false positive rates. In the pilot study discussed above this activity value was >3σ above the ISRE mean with the added criteria of NFκB activation being lower than 16 from the mean to identify IRF selective compounds. The ratio of ISRE:NFκB activity was also calculated as a selectivity index for prioritizing hit selection. Primary hits underwent secondary screening to determine the 50% effective concentration (EC50) and 50% cytotoxic concentration (CC50) of these agents using an 8-point dose-response curve in the human THP1-Dual cells. Cytotoxicity was measured in the plate using Cell Titer Glo (Promega). Identified candidate compounds were further screened (orthogonal screen) for cross species activity in the validated mouse J774-Dual assay using an 8-point dose-response format to determine EC50 and CC50 values as well as ISRE:NFκB ratios. A general process flow for the experimental design laid out above is depicted at FIG. 12.

As a specific example of conditions for the HTS detailed above, THP1 dual (InvivoGen) cell suspensions were cultured in RPMI 1640 (HyClone, Logan, Utah) supplemented with 10% fetal bovine serum (Hyclone), 100 U/mL Normocin (Invivogen), and 50 U/mL PenStrep between a density of 7·10⁵-2·10⁶ cells/mL. Every other passage, media was supplemented media with 5 U/mL Blasticidin and 100 U/mL Zeocin to maintain the expression constructs. Flat-bottom 384-well plates (Greiner, #781080) were seeded with 2.5·10⁴ cells/well in a total of 50 μL media containing 5 ng/mL phorbolmyristic acid (PMA) using a Matrix WellMate (Thermo Scientific) dispenser. After 48 hours of incubation at 37° C., media was removed using a BioTek ELx405 CW plate washer set to aspirate and wash each well three times with 65 μL 1×PBS. Plates were reloaded with 50 μL media without PMA and added treatments. Lipopolysaccharide in dimethylsulfoxide (DMSO) was transferred to control wells using a HP D300 microdispenser to a final concentration of 313 ng/mL. Compounds were copied from chemical library plates using a Sciclone (Caliper LifeSciences) mounted with a 384-pin transfer tool to obtain a final concentration of 10 μM. After incubation with treatments for 24 hours, 10 μL of supernatant media from each well was transferred into a white, flat-bottom 384-well plate for luminescence readout and a black-walled, clear flat-bottom 384-well plate for absorbance readout.

OTRADI Small Molecule Library

This library was purchased from SIGA Technologies in 2014 and is made up of compounds from the ChemBridge DIVERSet™-CL library, the ChemBridge CORE Library, and two focused antiviral libraries from Life Chemicals and ChemDiv.

Data Analysis and Statistics

The Dotmatics software suite (Dotmatics) was used for data analysis and storage. Raw data was uploaded to the system and evaluated using templates developed for the plate layout. Barcodes associated with each plate verify the identity of the dataset. To address “random” assay variability (unexplained variance in raw data across plates) normalization of data within each plate was performed by calculating percent of control (PC). This is necessary to enable comparison of results across plates or experiments and to allow a single cut-off for hit selection. The median and median absolute deviation (MAD) was utilized as statistical parameters as opposed to mean and standard deviation (SD), respectively, to diminish the effect of outliers on the final analysis results. The median positive control (χ_{P}) and median negative control (χ_{N}) was used to perform this in-plate normalization, with outliers in the positive and negative control sets being defined as 15% greater or less than the average value. The assay-specific in-plate positive and negative controls were used as the upper (100%) and lower (0%) bounds of the assay activity, and the activities of the test samples (Sᵢ) were calculated with respect to these values. This plate-wise normalization was used for the pilot validation screen described above. To determine if the data collected from each plate meet the minimum QC requirements, and if any patterns exist before and after data normalization, the distribution of control and test sample data was examined at the experiment-, plate- and well-level.

$$PC = 100 \times \frac{{S}_{i} - {χ}_{N}}{{χ}_{P} - {χ}_{N}} \qquad ( 1 )$$

Following data analysis and determination of PC (see equation 1 above) for each sample, visualization of data occurred in Vortex. This software was used to aggregate hits and further evaluate QC for each screening plate by observing the distribution of data points.

Results, Interpretations, and Limitations

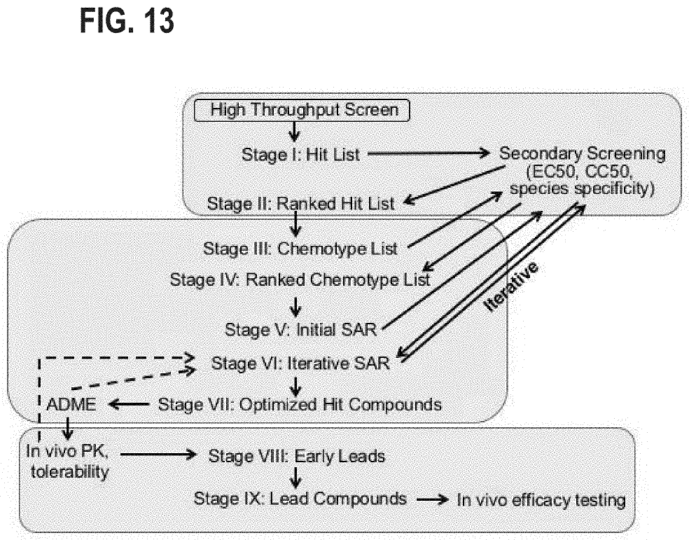

Following the THP1-Dual HTS it may be understood that over a hundred hit compounds with relatively equal known potency (Stage I: Hit List; FIG. 13) may be identified. These compounds then undergo secondary screening in THP1-Dual cells to determine EC50 and CC50 values and are further screened in the mouse cell line, J774-Dual, to assess cross species activity. The compounds are ranked based on these secondary screens and the ISRE:NFκB selectivity index (Stage II: Ranked Hit List; FIG. 13). Ranking priority is assessed in the following order: 1) high activity in human cells, 2) high ISRE:NFκB activity ratio in human cells, 3) low cytotoxicity, 4) activity in mouse cells. As discussed above, a goal is the identification of compounds demonstrating favorable activity in both the human and mouse screens, with a primary goal of developing a therapeutic agent for the treatment of stroke patients.

It may be understood that a caveat to using luciferase reporter-gene activation assays for HTS is that small molecule compounds have been identified that stabilize lucifierase resulting in luciferase activity that is actually independent of gene transcription thereby producing false positives. This potential problem was addressed via two approaches: 1) a counter-screen, in silico, was conducted on the compounds that pass the primary and secondary screens against a publicly available database (PubChem AID411) that has been compiled to flag compounds with known luciferase interactions, 2) confirmation of all positive compounds in a separate single reporter system using an ISRE coupled secreted embryonic alkaline phosphatase reporter cell line (THP1-Blue™ ISG cells, Invivogen) where the promoter used to drive the SEAP reporter is the same as in the primary screen. In the assay, the positive hits were identified based on >2σ signal activity over the background average measured with the ISRE-SEAP reporter. Therefore, any compounds that show positive ISRE-SEAP activity in this confirmation screen when the ISRE was coupled to SEAP were indicative of activity that is dependent on true ISRE driven gene transcription and were thus considered confirmed hits.

### Example 14

This example demonstrates evaluating and ranking lead compounds through hit to lead characterization.

Hits from stages I-II (FIG. 13) were evaluated and expanded through standard hit to lead optimization to maximize efficacy and minimize cytotoxicity. Compounds were profiled for ADME endpoints including: solubility, stability, protein binding and membrane permeability. The Ranked Hit List (see Stage II at FIG. 13) was further ranked and refined, and the compounds grouped based on structure similarities to define active chemotypes. Iterative Structure Activity Relationship (SAR) analysis was used to define key pharmacophores for each chemotype allowing optimization of compound structures. The most active compounds for each chemotype were synthesized and underwent ADME analysis to access potential in vivo activity.

Experimental Design

The compounds on the Ranked Hit List (Stage II, FIG. 13) were further ranked according to at least 1) potency, 2) perceived drugability (Lipinski's Rule of 5), and 3) synthetic accessibility. During this process the compounds were also grouped according to structure. The result was a list of starting point compounds/chemotypes (ranked as above; Stage III: Chemotype List; FIG. 13). Available chemical databases (e.g., Schrodinger Canvas Software and SciFinder) were searched for similar, commercially available compounds. These compounds then went back to the secondary screen (EC50, cytotoxicity and cross species activity, described above). These results allowed a re-ranking of the chemotypes (Stage IV: Ranked Chemotype List; FIG. 13). With a preliminary SAR with commercially available compounds in hand, a small library (3-5 compounds) for each of the top chemotypes was synthesized (Stage V: Initial SAR; FIG. 13). This Initial SAR was intended to define the pharmacophore (the active portion of the molecule) by paring away any unnecessary complexity. Chemotypes that lost activity with any structural variation were triaged. Once the pharmacophore was established, iterative structural variation commenced. This process was different for each chemotype, but it generally involved iterative variation of chemical substituents. Throughout the iterative SAR process, potency of new compounds was determined using the secondary screen (Stage VI: Iterative SAR; FIG. 13). From the iterative SAR the most potent and soluble (relative solubility determined by reverse phase HPLC) optimized hit compounds for each chemotype (Stage VII: Optimized Hit Compounds; FIG. 13) were identified. ADME profiling (solubility, stability, protein binding and permeability) was performed for each of the Optimized Hit Compounds to predict in vivo activity. Bioanalytical method development and ADME assays were performed with Cyprotex US or the OHSU Pharmacokinetics Core. In brief, a Liquid Chromatography-Mass Spectrometry/Mass Spectrometry (LC-MS/MS) method was developed for the compounds using standard MS conditions and HPLC columns. Solubility in buffer or media was determined at room temperature by optical density. Microsomal clearance (half-life, clearance, percent remaining) was determined as a measure of metabolic stability in human or mouse cells. Bidirectional permeability was measured using MDCK cells, with readouts including permeability rate coefficients and efflux ratios.

Results, Interpretations, and Limitations

Analysis of ADME characterization data resulted in additional SAR of compounds to minimize undesirable activity and/or maximize desirable activity. Any newly synthesized analogs were screened for activity and cytotoxicity (as above) and further screened through the ADME pipeline. In vitro potency and ADME profiles were used to rank compounds for in vivo testing (stages XIII-IX, FIG. 13).

### Example 15

This example demonstrates how the high throughput screening methodology discussed herein is conducted. A primary HTS was conducted in similar fashion as that described above at Example 13. All compounds were analyzed in batches of 40×384 well plates, which represents the number of plates performed in parallel during a given week of screening. The activity of each compound was evaluated via two different metrics, one relative to internal plate controls referred to as the percent activation (PA) method and the other relative to the average sample signal or the Z score method. The PA method takes into account the performance of each assay plate in terms of signal output and prevents inflation or absence of hits in plates where the signal strength of controls was significantly higher or lower than the average plate in a batch. Alternatively, the Z score method assumes the majority of compounds have no effect on signal and simply evaluates whether an individual compound output is outside of this background.

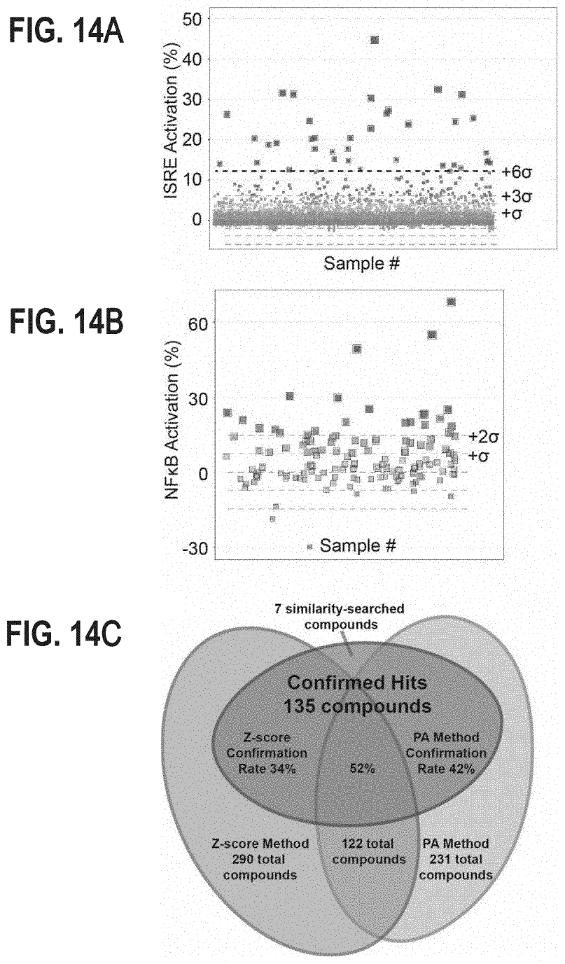

In the PA method, the average and standard deviation of the percent activation of the samples compared to internal controls was calculated. Compounds were assigned as hits if their percent ISRE activation exceeded 6σ above the mean for the batch. For the PA method, this resulted in 416 hits (0.25%). Of those 416 hits, 231 were identified as weak NFkB activators and prioritized for further screening (see FIG. 14C).

The Z score was calculated by subtracting the sample mean from individual sample signals and dividing by the standard deviation. A cut-off of Z score of ≥6 was used to identify ISRE activating compounds, which resulted in 478 hits (0.29%). Of those 478 hits, we identified 290 as weak NFkB activators and prioritized for further screening (see FIG. 14C).

FIG. 14A represents exemplary data from 40 primary HTS plates. In the PA method, the ISRE-coupled luminescence readout was normalized to control treatments on each plate to provide a relative percent activation score, as shown at FIG. 14A. Alternatively, in the Z score method, statistical analysis was performed on raw luminescence data. As discussed, queries greater than 66 from the batch baseline score mean were selected as hits.

As illustrated at FIG. 14B, a counterscreen was performed to select compounds with reduced NF-κB-coupled SEAP activation. The graph at FIG. 14B represents the percent NF-κB activation of all combined hits from the 40 primary HTS plates discussed with regard to FIG. 14A. Cutoffs for exclusion of hits with NF-κB activation above 2a were set based on their ISRE/NF-κB or ISRE-NF-κB signal scores.

Although both analysis methods identified a similar number of hits, only 122 compounds (˜30%) were shared between the two methods (FIG. 14C). As the compounds identified in the primary HTS were tested at a single dose and not replicated, a validation screen was performed on compounds identified as hits in either one of the analysis methods. This gave a combined unique hit count of 399 or 0.24% of queried compounds to carry forward for reevaluation. The individual hit samples from the original library plates were “cherry-picked” and were combined into two new compound plates for further screening. Interestingly, a review of the chemical structures of the hits identified at least 4 distinct clusters of compounds with similar scaffolds and chemical features. Similarity searches of additional unscreened chemical library holdings were performed which identified 98 more compounds with related structures. These compounds were added to the new cherry-picked hit compound plates for the validation screen.

Cherry-Picked and Similarity Searched Compound Activity

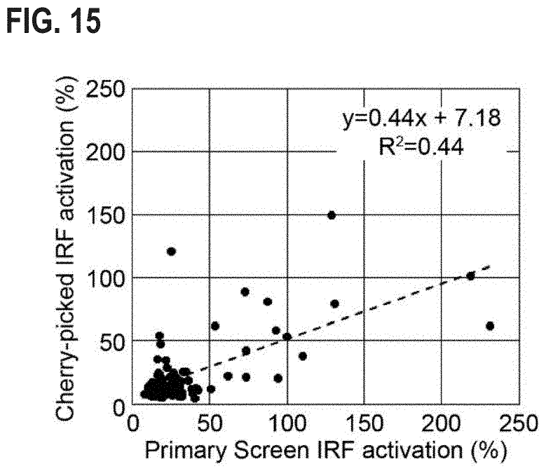

ISRE and NFkB activation of the cherry-picked compounds was measured in duplicate for the human macrophage model used in the primary screen. In addition, the compounds were screened in duplicate against a mouse macrophage model with identical reporter constructs to determine which compounds have cross-species activity. Blank wells distributed across the cherry-picking plates were included for background to develop the cut-offs for confirming hits. A cut-off for ISRE activation was again set at 66 above the background average and 2σ for evaluation of NFkB activation for the counterscreen. A fold (ISRE/NFkB) and difference (ISRE-NFkB) score was calculated for hits with SEAP activation of greater than 2σ. Of the 497 compounds tested in the cherry-picking effort, 142 total confirmed ISRE activating hits were identified in the human line. The majority of confirmed compounds showed similar percent activation between the cherry-picking and original primary screen output (FIG. 15). Specifically, FIG. 15 depicts correlation between the IRF activation observed in the cherry-picking confirmation screen and the singlicate primary screen measurement.

Of these 142 compounds, 135 are original hits from the primary screen and the other 7 are from similarity searches. These 7 compounds hits are significant in that it demonstrates that the identified chemical families from primary screening have viable information about features critical to activity in the system. It was determined that the overall confirmation rate of the PA method is 42% and the Zscore method is 34%. Compounds that were identified as hits in both methods had a confirmation rate of 52%. Sixty-nine of the confirmed ISRE active compounds also demonstrated low NFkB activation.

Cross-Species Evaluation and Orthogonal Screen

Importantly, of these 69 compounds, 5 were positive for activity in the mouse macrophage model, indicating that they possess cross-species activity. Having cross-species active hits greatly increases the chances of developing leads that can be evaluated in the efficacy model of stroke in mice, and for enhancing the ability to continue progression into the drug development pipeline.

The identified compounds from the HTS described above were compared, resulting in the identification of 4 major chemofamilies of compounds based on structure. Initial SAR included commercially available and synthesized analogs of these chemofamilies to define structure activity relationships for therapeutic development. ¹H NMR was recorded on a Bruker DPX spectrometer at 400 MHz. Chemical shifts are reported as parts per million (ppm) downfield from an internal tetramethylsilane standard or solvent references. For air- and water-sensitive reactions, glassware was oven-dried prior to use and reactions were performed under argon. Dichloromethane, dimethylformamide, and tetrahydrofuran were dried using the solvent purification system manufactured by Glass Contour, Inc. (Laguna Beach, Calif.). All other solvents were of ACS chemical grade (Fisher Scientific) and used without further purification unless otherwise indicated. Analytical thin-layer chromatography was performed with silica gel 60 F₂₅₄ glass plates (SiliCycle). Flash column chromatography was conducted with either pre-packed Redisep R_{f} normal/reverse phase columns (Biotage).

### Example 16

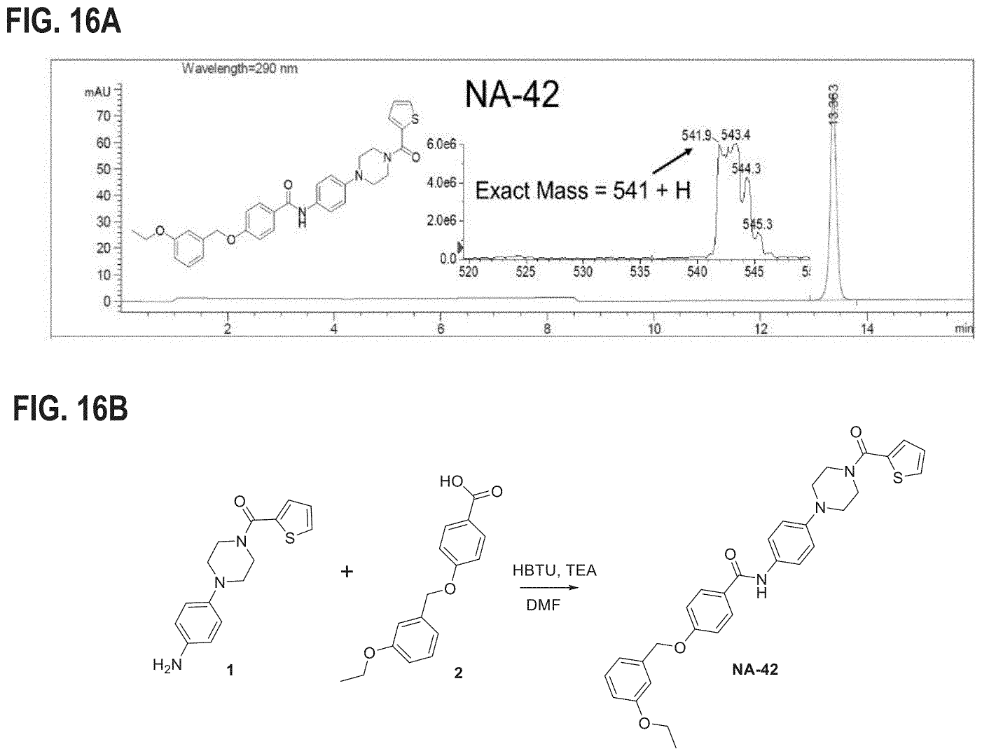

This example demonstrates an example compound identified via the high throughput screening methodology of the present disclosure. A chemofamily, denoted NA, was found via the HTS discussed herein, which activates IRF3 in both mouse and human cells. One preferred example is NA-42, or 4-((3-ethoxybenzyl)oxy)-N-(4-(4-(thiophene-2-carbonyl)piperazin-1-yl)phenyl)benzamide. 4-((3-ethoxybenzyl)oxy)benzoic acid (2) (54 mg, 0.2 mmol) dissolved in 5 mL DMF was slowly added HBTU (151 mg, 0.4 mmol) and TEA (60 mg, 0.6 mmol) and stirred at room temperature for 30 min. Then, (4-(4-aminophenyl)piperazin-1-yl)(thiophen-2-yl)methanone (1) (57 mg, 0.2 mmol) was introduced into the solution. The whole mixture was stirred at 60° C. overnight. The reaction mixture was diluted with ethyl acetate (20 mL), washed with water (2×25 mL) and brine (2×25 mL), dried over MgSO₄, and concentrated. The crude product was purified by flash column chromatography (50% ethyl acetate in hexanes) to yield NA-42 (27.6 mg, 52%). 1H NMR (CDCl3, 400 MHz): δ 1.44 (t, 3H), 3.21 (m, 4H), 3.96 (m, 4H), 4.1 (q, 2H), 5.11 (s, 2H), 7.12-6.85 (m, 8H), 7.36 (m, 2H), 7.65-7.54 (m, 3H), 7.75 (s, 1H), 7.85 (m, 2H). FIG. 16A depicts a chromatograph of NA-42, along with its chemical structure and mass, and FIG. 16B depicts the reaction scheme for synthesis of NA-42.

### Example 17

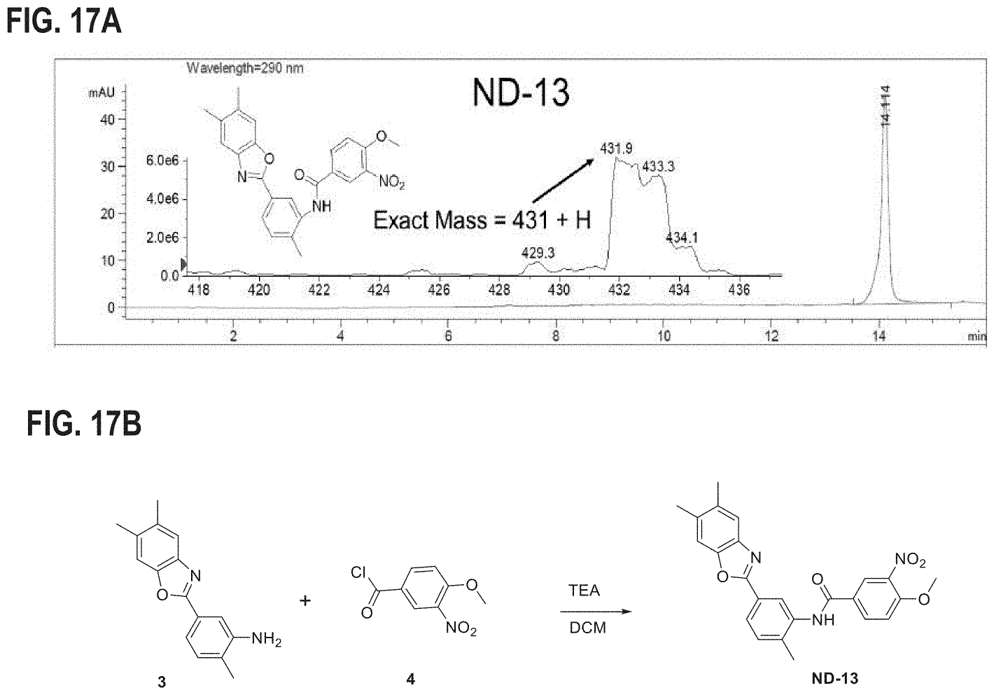

This example demonstrates another example compound identified via the high throughput screening methodology of the present disclosure. A chemofamily, denoted ND, was found via the HTS discussed herein, which activates IRF3 in both mouse and human cells. One preferred example is ND-13, or N-(5-(5,6-dimethylbenzo[d]oxazol-2-yl)-2-methylphenyl)-4-methoxy-3-nitrobenzamide. 5-(5,6-dimethylbenzo[d]oxazol-2-yl)-2-methylaniline (3) (126.0 mg, 0.5 mmol) in dry DCM (10 mL) was slowly added 4-methoxy-3-nitrobenzoyl chloride (4) (108 mg, 0.5 mmol) and triethylamine (50.0 mg, 0.5 mmol). The whole mixture was stirred at 40° C. for 6 hr until the starting material disappeared, determined by TLC. After the reaction was over, 10 mL DCM and 5 mL water was added into the solution. The organic layer was extracted and dried over MgSO4, and concentrated. The crude product was purified by flash column chromatography (20% ethyl acetate in hexanes) to yield compound ND-13 (143 mg, 65% yield). 1H NMR (CDCl3, 400 MHz): δ 2.32 (m, 9H), 4.01 (s, 3H), 6.78 (m, 2H), 7.01 (s, 1H), 7.12 (s, 1H), 7.26 (m, 1H), 7.85 (m, 1H), 8.05 (s, 1H), 8.12 (m, 1H) 8.35 (s, 1H). FIG. 17A depicts a chromatograph of ND-13, along with is chemical structure and mass, and FIG. 17B depicts the reaction scheme for synthesis of ND-13.

### Example 18

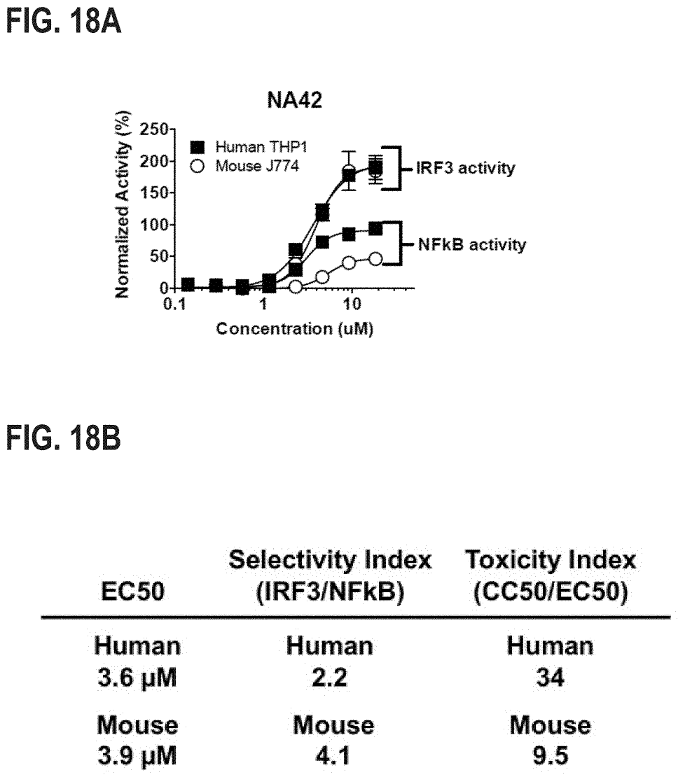

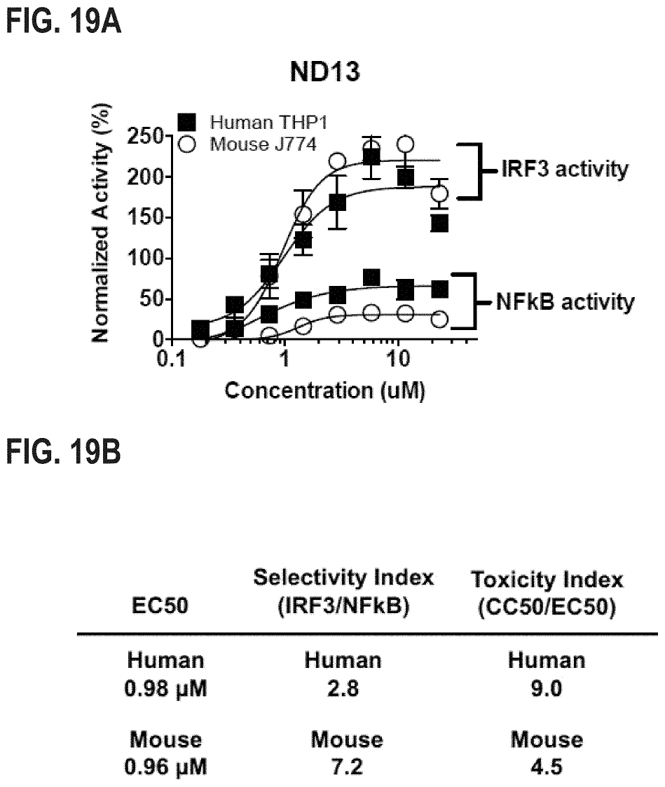

This example demonstrates various parameters assessed with regard to two of the compounds identified in the high-throughput screen methodology of the present disclosure. IRF3 and NF-κB activity was determined in both human THP1 cells and mouse J774 cells via the use of the NF-κB-SEAP and IRF-Lucia luciferase dual reporter as discussed above, for chemical entities corresponding to both the ND and NA chemofamilies. Depicted at FIG. 18A is an example 8-point dose response curve showing reporter construct activity in the human and mouse macrophage-like cells for NA-42. The data shown at FIG. 18A illustrates high IRF3 activity and low off-target NF-κB activation for NA-42. Depicted at FIG. 18B are values corresponding to EC50, selectivity index (IRF3 activity/NF-κB activity), and toxicity index (CC50/EC50) for NA-42. Table 17 shows values obtained with regard to IRF EC50, NF-κB EC50, CC50, % IRF activation at peak, and % NF-κB activation at peak for each compound of the NA chemofamily and NC chemofamily in both the human and mouse reporter cell lines. Along similar lines, depicted at FIG. 19A is an example 8-point dose response curve showing reporter construct activity in the human and mouse macrophage-like cells for ND-13. The data shown at FIG. 19A illustrates high IRF3 activity and low off-target NF-κB activation for ND-13. Depicted at FIG. 19B are values corresponding to EC50, selectivity index (IRF3 activity/NF-κB activity), and toxicity index (CC50/EC50) for ND-13. Table 18 shows values obtained with regard to IRF EC50, NF-κB EC50, CC50, % IRF activation at peak, and % NF-κB activation at peak for the ND chemofamily and NB chemofamily in both the human and mouse reporter cell lines.

TABLE 17
Various parameters obtained for compounds of the NA and NC chemofamilies

| | Human Reporter Cell Line | | | | | Mouse Reporter Cell Line | | | | |
|---|---|---|---|---|---|---|---|---|---|---|
| | | | | THP1 | THP1 | | | | J774 | J774 |
| Com- | THP1 | THP1 | Thp1 | IRF | NFkB | J774 | J774 | J774 | IRF | NFkB |
| pound | IRF | NFkB | cytotox | % | % | IRF | NFkB | cytotox | % | % |
| common | EC50 | EC50 | CC50 | activation | activation | EC50 | EC50 | CC50 | activation | activation |
| name | (uM) | (uM) | (uM) | at peak | at peak | (uM) | (uM) | (uM) | at peak | at peak |
| NA42 | 3.598 | 3.036 | 121 | 197.6 | 91.01 | 3.899 | 5.356 | 37.17 | 191.2 | 47.1 |
| (100% | | | | | | | | | | |
| pure) | | | | | | | | | | |
| NA-21 | 0.73 | 1.08 | 39.39 | 166.90 | 61.87 | 1.01 | 1.05 | 63.77 | 38.86 | 3.87 |
| NA-24 | 3.14 | 4.16 | 16.84 | 137.4 | 52.74 | 4.672 | — | 36.23 | 111.2 | — |
| NA-59 | ~2.529 | ~2.574 | 15.96 | 105 | 66.37 | 3.086 | 4.365 | 36.12 | 150.5 | 24.83 |
| NA-1 | 1.94 | 2.07 | 10.45 | 116.20 | 79.93 | ~3.062 | ~3.188 | 13.67 | 67.54 | 10.59 |
| 90% | | | | | | | | | | |
| NA-20 | 1.941 | 2.762 | 11.78 | 78.49 | 26.29 | 4.186 | ~4.751 | 43.58 | 29.98 | 6.987 |
| NA-39 | 7.024 | 7.905 | >25 uM | 94.34 | 35.37 | 9.808 | — | >25 uM | 77.89 | — |
| NA-32 | 1.427 | 3.126 | 36.62 | 162.5 | 101.4 | 11.87 | — | 67.24 | 77.58 | — |
| NA-41 | 10.67 | 12.46 | >25 uM | 57.87 | 42.38 | 10.56 | — | >25 uM | 37.71 | — |
| NA13 | ~1.555 | 1.219 | >25 uM | 121.4 | 49.98 | 0.8206 | ~2.860 | >25 uM | 3.434 | 6.052 |
| NA-60 | 2.23 | 2.27 | 22.99 | 166.8 | 59.45 | 1.77 | — | — | 33.91 | — |
| NA-25 | 3.292 | 4.995 | 35.47 | 121.6 | 18.01 | 4.376 | 8.147 | >25 uM | 18.01 | 31.46 |
| NA-33 | 1.845 | 1.62 | 9.583 | 82.09 | 21.91 | 2.636 | 3.812 | 25.51 | 15.46 | 5.323 |
| NA-6 | 7.471 | — | >25 uM | 26.39 | — | 13.12 | — | >25 uM | 22.78 | — |
| NA-14 | ~2.619 | ~2.419 | >25 uM | 52.61 | 13.09 | 5.23 | — | >25 uM | 4.471 | — |
| NA-7 | 3.914 | — | >25 uM | 38.82 | — | 4.359 | — | >25 uM | 5.638 | — |
| NA-44 | 4.452 | 0.92 | 50.3 | 37.95 | 6.35 | 7.38 | 7.647 | >25 uM | 2.085 | 7.371 |
| NA-30 | 2.684 | 1.053 | 18.78 | 24.95 | 3.991 | ~3.33 | — | 40.74 | 2.95 | — |
| NA-19 | 1.018 | 1.472 | 8.487 | 167.1 | 100.2 | — | — | >25 uM | — | — |
| NA-34 | 4.416 | 6.381 | >25 uM | 94.93 | 40.55 | — | — | >25 uM | — | — |
| NA-23 | 1.707 | ~2.307 | 12.69 | 67.51 | 28.58 | — | ~2.527 | 22.11 | — | 2.067 |
| NA-51 | ~2.633 | — | 11.35 | 59.26 | — | — | — | >25 uM | — | — |
| NA-53 | ~1.477 | — | 12.31 | 24.36 | — | — | — | 29.23 | — | — |
| NA-16 | 6.46 | ~5.229 | >25 uM | 25.5 | 12.07 | — | 12.91 | >25 uM | — | 5.524 |
| NA-15 | 5.89 | — | >25 uM | 19.84 | — | — | 1.628 | >25 uM | — | 7.696 |
| NA-50 | ~2.504 | — | 13.1 | 19.98 | — | — | — | >25 uM | — | — |
| NA-71 | 3.325 | 6.566 | >10 uM | 14.96 | 19.55 | — | — | >10 uM | — | — |
| NA-17 | 2.402 | — | 14.64 | 5.264 | — | — | — | >25 uM | — | — |
| NA-18 | 3.076 | — | >25 uM | 3.616 | — | — | — | >25 uM | — | — |
| NA-38 | ~11.15 | 3.459 | >25 uM | 9.165 | 10.68 | ~10.2 | — | >25 uM | 2.98 | — |
| NA-46 | 0.1088 | — | >25 uM | 3 | — | — | — | >25 uM | — | — |
| NA-47 | 0.3585 | — | >25 uM | 5.445 | — | — | — | >25 uM | — | — |
| NA-4 | ~13.24 | — | 37.84 | 6.807 | — | — | — | >25 uM | — | — |
| NA-22 | — | ~2.618 | >25 uM | — | 11.75 | — | — | >25 uM | — | — |
| NA-2 | — | — | >25 uM | — | — | — | — | >25 uM | — | — |
| NA-3 | — | — | >25 uM | — | — | — | — | >25 uM | — | — |
| NA-5 | — | — | >25 uM | — | — | — | — | >25 uM | — | — |
| NA-8 | — | — | >25 uM | — | — | — | — | >25 uM | — | — |
| NA-9 | — | — | >25 uM | — | — | — | — | >25 uM | — | — |
| NA-10 | — | — | >25 uM | — | — | — | — | >25 uM | — | — |
| NA-11 | — | — | >25 uM | — | — | — | — | >25 uM | — | — |
| NA-12 | — | — | >25 uM | — | — | — | — | >25 uM | — | — |
| NA-26 | — | — | >25 uM | — | — | — | — | >25 uM | — | — |
| NA-27 | — | — | >25 uM | — | — | — | — | >25 uM | — | — |
| NA-28 | — | 3.02 | >25 uM | — | 11.38 | — | — | >25 uM | — | — |
| NA-29 | — | — | 64.95 | — | — | — | — | 45.83 | — | — |
| NA-31 | — | ~3.243 | >25 uM | — | 15.09 | — | — | >25 uM | — | — |
| NA-35 | — | — | — | — | — | — | — | — | — | — |
| NA-36 | — | — | >25 uM | — | — | — | — | >25 uM | — | — |
| NA-37 | — | — | >25 uM | — | — | — | — | >25 uM | — | — |
| NA-40 | — | — | >25 uM | — | — | — | — | >25 uM | — | — |
| NA-43 | ~10.81 | — | >25 uM | 9.137 | — | — | — | >25 uM | — | — |
| NA-45 | — | — | 10.68 | — | — | — | — | >25 uM | — | — |
| NA-48 | — | — | >25 uM | — | — | — | — | >25 uM | — | — |
| NA-49 | — | — | 27.85 | — | — | — | — | >25 uM | — | — |
| NA-52 | 1.417 | — | >25 uM | 2.874 | — | — | — | >25 uM | — | — |
| NA-54 | — | — | >25 uM | — | — | — | — | >25 uM | — | — |
| NA-55 | — | — | >25 uM | — | — | — | — | >25 uM | — | — |
| NA-56 | — | — | >25 uM | — | — | — | — | >25 uM | — | — |
| NA-57 | — | — | >25 uM | — | — | — | — | >25 uM | — | — |
| NA-58 | — | — | >25 uM | — | — | — | — | >25 uM | — | — |
| NA-61 | 2.27 | — | — | 14.37 | — | — | — | — | — | — |
| NA-62 | — | — | — | — | — | — | — | — | — | — |
| NA-63 | — | 2.94 | — | — | 30.78 | — | 2.77 | 73.86 | — | 34.77 |
| NA-65 | — | — | >10 uM | — | — | — | — | >10 uM | — | — |
| NA-66 | — | — | >10 uM | — | — | — | — | >10 uM | — | — |
| NA-67 | — | — | >10 uM | — | — | — | — | >10 uM | — | — |
| NA-68 | — | — | >10 uM | — | — | — | — | >10 uM | — | — |
| NA-69 | — | — | >10 uM | — | — | — | — | >10 uM | — | — |
| NA-70 | — | — | >10 uM | — | — | — | — | >10 uM | — | — |
| NA-72 | — | — | >10 uM | — | — | — | — | >10 uM | — | — |
| NA-73 | — | — | >10 uM | — | — | — | — | >10 uM | — | — |
| NA-74 | — | — | >10 uM | — | — | — | — | >10 uM | — | — |
| NC-1 | 2.55 | — | 27.38 | 45.71 | — | — | — | >25 uM | — | — |
| NC-2 | 7.984 | 15.4 | >25 uM | 70.6 | 24.39 | 14.58 | — | >25 uM | 4.6 | — |
| NC-3 | 4.131 | ~6.132 | >25 uM | 48.47 | 15.04 | — | — | >25 uM | — | — |
| NC-4 | 9.885 | — | >25 uM | 37.07 | — | ~5.878 | — | >25 uM | 6.428 | — |

TABLE 18
Various parameters obtained for compounds of the ND and NB chemofamilies

| | Human Reporter Cell Line | | | | | Mouse Reporter Cell Line | | | | |
|---|---|---|---|---|---|---|---|---|---|---|
| | | | | THP1 | THP1 | | | | J774 | J774 |
| Com- | THP1 | THP1 | Thp1 | IRF | NFkB | J774 | J774 | J774 | IRF | NFkB |
| pound | IRF | NFkB | cytotox | % | % | IRF | NFkB | cytotox | % | % |
| common | EC50 | EC50 | CC50 | activation | activation | EC50 | EC50 | CC50 | activation | activation |
| name | (uM) | (uM) | (uM) | at peak | at peak | (uM) | (uM) | (uM) | at peak | at peak |
| ND13 | 0.975 | 0.5559 | 8.761 | 188.1 | 66.61 | 0.9556 | 1.318 | 4.323 | 220.5 | 30.71 |
| (100% | | | | | | | | | | |
| pure) | | | | | | | | | | |
| ND-95 | 0.5899 | 1.273 | 26.65 | 52.24 | 37.69 | 4.103 | 3.006 | >25 uM | 98.11 | 13.16 |
| ND52 | 3.888 | ~5.508 | 27.61 | 100.4 | 41.23 | 5.867 | 7.533 | 100.8 | 131.7 | 14.17 |
| (100% | | | | | | | | | | |
| pure) | | | | | | | | | | |
| ND-40 | 2.627 | — | 16.11 | 125.2 | — | 4.5 | — | >25 uM | 119.6 | — |
| ND-16 | 3.124 | 3.245 | 32.73 | 114.7 | 40.23 | 3.581 | 6.755 | >25 uM | 83.02 | 11.43 |
| ND-2 | 1.12 | 4.47 | 27.56 | 38.58 | 3.85 | 4.81 | 11.40 | 65.76 | 141.80 | 21.17 |
| ND-38 | 4.293 | ~3.125 | 44.9 | 54.94 | 14.2 | 7.667 | 11.99 | >25 uM | 114 | 7.782 |
| ND-1 | ~12.36 | ~12.66 | >25 uM | 45.13 | 21.77 | 8.818 | 8.75 | >25 uM | 28.40 | 3.93 |
| 90% | | | | | | | | | | |
| ND-80 | 6.164 | ~6.552 | 33.94 | 38.49 | | ~10.08 | — | >25 uM | 26.88 | — |
| ND-6 | 11.90 | | 49.76 | 29.22 | | ~7.27 | | >25 uM | 18.79 | |
| ND-5 | 14.71 | 12.35 | >25 uM | 29.74 | 12.44 | ~12.94 | — | >25 uM | 12.45 | — |
| ND-94 | 32.11 | ~23.55 | >25 uM | 99.06 | ~29.40 | 4.525 | — | >25 uM | 5.09 | — |
| ND-48 | ~3.052 | — | 7.261 | 7.207 | — | 10.04 | — | >25 uM | 4.4 | — |
| ND-3 | 3.14 | — | 30.69 | 46.30 | — | — | — | >25 uM | — | — |
| ND-4 | 3.13 | — | 49.34 | 40.06 | — | — | — | >25 uM | — | — |
| ND-7 | 8.42 | 6.00 | >25 uM | 22.39 | 9.18 | — | 30.56 | >25 uM | — | 13.05 |
| ND-8 | 2.948 | — | >25 uM | 47.45 | — | — | — | >25 uM | — | — |
| ND-65 | 5.391 | 12 | >25 uM | 44.01 | 61.62 | — | — | >25 uM | — | — |
| ND-18 | 13.49 | — | — | 40.51 | — | — | — | — | — | — |
| ND-67 | 0.4012 | ~12.26 | 8.866 | 12.33 | 8.298 | — | — | >25 uM | — | — |
| ND-22 | 3.827 | | 9.795 | 10.23 | | — | — | — | — | — |
| ND-30 | 15.21 | — | >25 uM | 19.39 | — | — | — | >25 uM | — | — |
| ND-31 | ~3.143 | — | 22.32 | 12.66 | — | — | — | >25 uM | — | — |
| ND-34 | ~11.39 | — | >25 uM | 5.084 | — | — | — | >25 uM | — | — |
| ND-35 | 2.134 | — | 35.73 | 18.31 | — | — | — | >25 uM | — | — |
| ND-41 | 2.045 | — | >25 uM | 7.946 | — | — | — | >25 uM | — | — |
| ND-76 | ~0.7161 | — | >25 uM | 18.25 | — | — | — | >25 uM | — | — |
| ND-89 | ~0.4962 | 10.59 | >25 uM | 11.76 | 32.43 | — | — | >25 uM | — | — |
| ND-42 | ~0.002245 | ~15.61 | >25 uM | 6.8 | 4.698 | — | — | >25 uM | — | — |
| ND-43 | 5.099 | — | 20.27 | 21.84 | — | — | — | >25 uM | — | — |
| ND-37 | 11.84 | — | >25 uM | 10.09 | — | — | — | >25 uM | — | — |
| ND-50 | ~11.94 | — | >25 uM | 33.22 | — | — | — | >25 uM | — | — |
| ND-64 | 7.267 | — | >25 uM | 9.187 | — | — | — | >25 uM | — | — |
| ND-58 | 2.803 | — | >25 uM | 29.05 | — | — | — | >25 uM | — | — |
| ND-60 | ~6.308 | — | >25 uM | 7.037 | — | — | — | >25 uM | — | — |
| ND-69 | ~5.393 | — | >25 uM | 6.518 | — | — | — | >25 uM | — | — |
| ND-70 | 4.759 | ~4.325 | 17.45 | 5.799 | 9.252 | — | — | >25 uM | — | — |
| ND-102 | 24.68 | 57.93 | — | 29.39 | 50 | — | — | — | — | — |
| ND-71 | 8.488 | 6.943 | >25 uM | 16.75 | 36.38 | — | — | >25 uM | — | — |
| ND-98 | 3.026 | — | 18.05 | 28.11 | — | — | — | 19.32 | — | — |
| ND-47 | 10.58 | 2.102 | >25 uM | 18.91 | 17.32 | — | 9.71 | >25 uM | — | 9.409 |
| ND-9 | — | — | >25 uM | — | — | — | — | >25 uM | — | — |
| ND-10 | — | — | >25 uM | — | — | — | — | >25 uM | — | — |
| ND-11 | — | — | >25 uM | — | — | — | — | >25 uM | — | — |
| ND-12 | — | — | 47.6 | — | — | — | — | >25 uM | — | — |
| ND-14 | — | — | >25 uM | — | — | — | — | >25 uM | — | — |
| ND-15 | — | — | >25 uM | — | — | — | — | >25 uM | — | — |
| ND-17 | — | — | >25 uM | — | — | — | — | >25 uM | — | — |
| ND-19 | — | — | — | — | — | — | — | — | — | — |
| ND-20 | — | — | — | — | — | — | — | — | — | — |
| ND-21 | | — | — | | — | | — | — | | — |
| ND-23 | — | — | — | — | — | — | — | — | — | — |
| ND-24 | — | | — | — | | — | — | — | — | — |
| ND-25 | — | — | — | — | — | — | — | — | — | — |
| ND-26 | — | — | — | — | — | — | — | — | — | — |
| ND-27 | 4.683 | — | >25 uM | 2.797 | — | — | — | >25 uM | — | — |
| ND-28 | ~3.299 | — | >25 uM | 1.632 | — | — | — | >25 uM | — | — |
| ND-29 | — | — | >25 uM | — | — | — | — | >25 uM | — | — |
| ND-32 | — | — | >25 uM | — | — | — | — | >25 uM | — | — |
| ND-33 | — | — | >25 uM | — | — | — | — | >25 uM | — | — |
| ND-36 | ~8.051 | — | >25 uM | 2.642 | — | — | — | >25 uM | — | — |
| ND-39 | 2.49 | — | >25 uM | 2.701 | — | — | — | >25 uM | — | — |
| ND-44 | — | — | >25 uM | — | — | — | — | >25 uM | — | — |
| ND-45 | — | — | 12.88 | — | — | — | — | >25 uM | — | — |
| ND-46 | 3.224 | — | 13.1 | 2.977 | — | — | — | 27.89 | — | — |
| ND-49 | — | — | 5.516 | — | — | — | — | >25 uM | — | — |
| ND-51 | — | — | 22.11 | — | — | — | — | >25 uM | — | — |
| ND-53 | — | — | >25 uM | — | — | — | — | >25 uM | — | — |
| ND-54 | — | — | 6.843 | — | — | — | — | >25 uM | — | — |
| ND-55 | — | — | >25 uM | — | — | — | — | >25 uM | — | — |
| ND-56 | — | — | >25 uM | — | — | — | — | >25 uM | — | — |
| ND-57 | — | — | >25 uM | — | — | — | — | >25 uM | — | — |
| ND-59 | — | — | >25 uM | — | — | — | — | >25 uM | — | — |
| ND-61 | — | ~5.613 | >25 uM | — | 38.9 | — | — | >25 uM | — | — |
| ND-62 | — | ~9.342 | >25 uM | — | 31.3 | — | — | >25 uM | — | — |
| ND-63 | — | ~3.198 | 27.45 | — | 22.04 | — | — | >25 uM | — | — |
| ND-66 | — | ~0.6596 | 17.88 | — | 20.12 | ~28.57 | — | >25 uM | ~32.86 | — |
| ND-68 | — | ~9.752 | >25 uM | — | 26.9 | — | — | >25 uM | — | — |
| ND-72 | — | — | >25 uM | — | — | — | — | >25 uM | — | — |
| ND-73 | — | — | >25 uM | — | — | — | — | >25 uM | — | — |
| ND-74 | — | 32.52 | >25 uM | — | 34.31 | — | — | >25 uM | — | — |
| ND-75 | — | — | >25 uM | — | — | — | — | >25 uM | — | — |
| ND-77 | — | — | >25 uM | — | — | — | — | >25 uM | — | — |
| ND-78 | — | — | >25 uM | — | — | — | — | >25 uM | — | — |
| ND-79 | — | ~11.27 | >25 uM | — | 30.47 | — | — | >25 uM | — | — |
| ND-81 | — | — | >25 uM | — | — | ~9.79 | — | >25 uM | 23.19 | — |
| ND-82 | — | — | >25 uM | — | — | ~9.356 | — | >25 uM | 22.57 | — |
| ND-83 | — | — | >25 uM | — | — | — | — | >25 uM | — | — |
| ND-84 | ~19.62 | — | >25 uM | 5.898 | — | — | — | >25 uM | — | — |
| ND-85 | — | — | >25 uM | — | — | — | — | >25 uM | — | — |
| ND-86 | — | ~3.855 | >25 uM | — | 11.09 | — | — | >25 uM | — | — |
| ND-87 | — | — | 3.021 | — | — | — | — | >25 uM | — | — |
| ND-88 | — | 1.716 | >25 uM | — | 62.16 | — | 0.72 | >25 uM | — | 72.85 |
| ND-90 | — | — | >25 uM | — | — | — | — | >25 uM | — | — |
| ND-91 | ~5.874 | — | >25 uM | 6.13 | — | — | — | >25 uM | — | — |
| ND-92 | — | ~2.335 | >25 uM | 8.193 | — | — | — | >25 uM | — | — |
| ND-93 | — | — | >25 uM | — | — | — | — | >25 uM | — | — |
| ND-96 | ~4.098 | ~4.092 | 30.06 | 2.617 | 30.01 | 18.88 | — | >25 uM | 5.389 | — |
| ND-97 | — | — | >25 uM | — | — | — | — | >25 uM | — | — |
| ND-99 | — | — | >25 uM | — | — | — | — | >25 uM | — | — |
| ND-100 | — | — | >25 uM | — | — | — | — | >25 uM | — | — |
| ND-101 | — | ~3.754 | >25 uM | — | 5.177 | — | — | >25 uM | — | — |
| ND-103 | — | 25 | — | — | 24.6 | — | 9.17 | — | — | 16.98 |
| ND-104 | — | 12.22 | — | — | 25.66 | — | — | — | — | — |
| ND-105 | — | 2.193 | >10 | 15.2 | — | — | — | >10 | — | — |
| ND-106 | — | — | >10 | — | — | — | — | >10 | — | — |
| ND-107 | — | — | >10 | — | — | — | — | >10 | — | — |
| ND-108 | — | — | >10 | — | — | — | — | >10 | — | — |
| NB-18 | 2.2342 | 3.656 | >25 uM | 70.3 | 42.55 | ~ 4.84 | — | >25 uM | 2.303 | — |
| NB-1 | 2.532 | 2.542 | 32.8 | 115.50 | 45.65 | 6.565 | 19.91 | >111 | 54.52 | 9.57 |
| NB-22 | 3.257 | — | >25 uM | 12.98 | — | 64.99 | — | >25 uM | 24.91 | — |
| NB-23 | 3.266 | 4.588 | 30.48 | 85.8 | 44.36 | 6.559 | 9.532 | 36.87 | 72.79 | 7.102 |
| NB-8 | 4.7 | 13.88 | >25 uM | 94.8 | 64.73 | 5.673 | — | >25 uM | 52.86 | — |
| NB-17 | 6.0658 | ~3.14 | >25 uM | 4.702 | 7.174 | 8.677 | 8.795 | >25 uM | 16.67 | 6.874 |
| NB-14 | 6.899 | — | >25 uM | 36.49 | — | — | 11.26 | >25 uM | — | 1.923 |
| NB-3 | ~1.567 | — | 52.49 | 31 | 38 | 8.539 | — | >25 uM | 2.603 | — |
| | | | | | (@25 uM) | | | | | |
| NB-4 | ~72.07 | — | >25 uM | 5.61 | — | — | — | >25 uM | — | — |
| | | | | (@25 uM) | | | | | | |

### Example 19

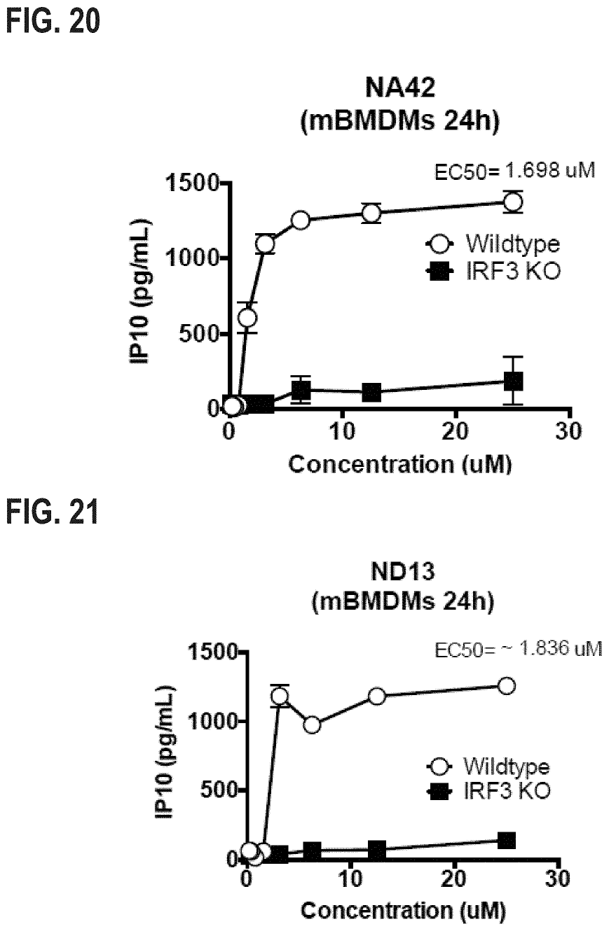

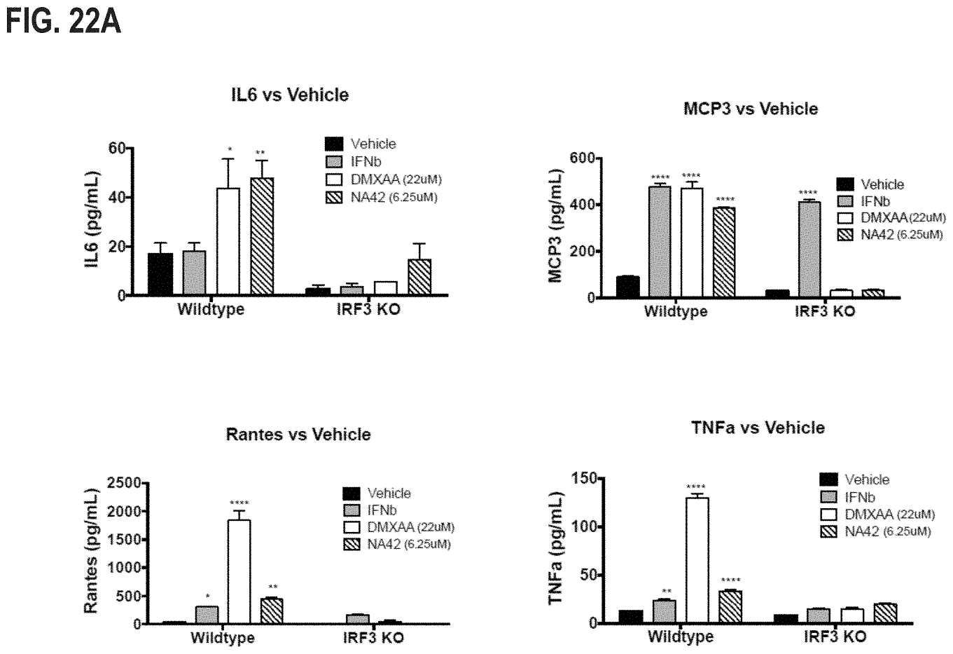

This example demonstrates target specificity in mice of compounds identified in the high-throughput screening methodology of the present disclosure. Target specificity of chemical entities corresponding to the ND and NA chemofamiles was examined by determining dose-dependent expression of IP10, a highly inducible, primary response gene that belongs to the C-X-C chemokine superfamily. As one representative example, FIG. 20 illustrates IRF3 dependent induction of IP-10 in mouse bone marrow derived macrophages (mBMDMs), as a function of increasing concentrations of NA-42 (compare IP-10 induction in wild-type cells with IRF3 knock-out cells). Along similar lines, FIG. 21 illustrates IRF3 dependent induction of IP-10 in mBMDMs as a function of increasing concentrations of ND-13. IP-10 expression was monitored by a mouse CXCL10/IP10 ELISA (Invitrogen, Carlsbad, Calif.), and is expressed at FIG. 20 and FIG. 21 as pg/ml.

### Example 20

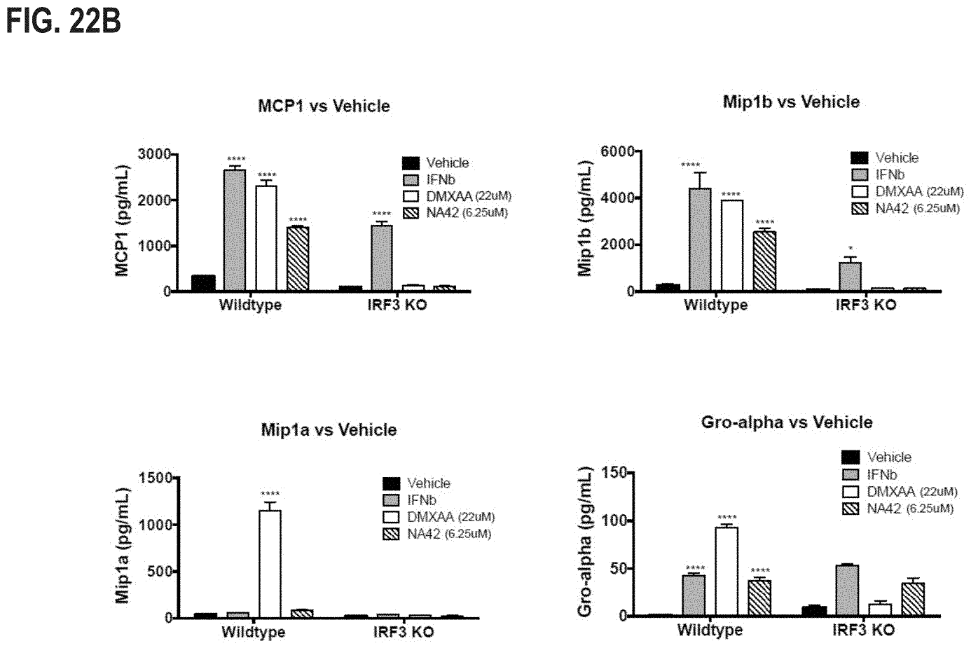

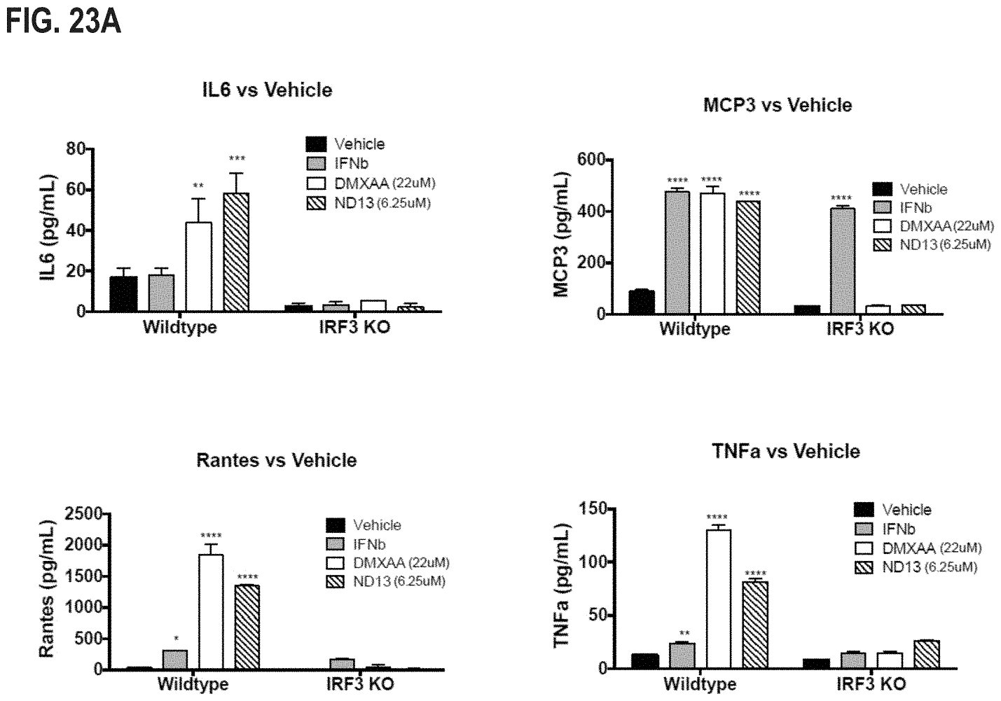

This example demonstrates IRF3 dependent cytokine induction in vitro by compounds identified via the high-throughput screening methodology of the present disclosure. IRF3 dependent cytokine induction by chemical entities corresponding to the ND and NA chemofamiles was examined by monitoring expression of various cytokines in mBMDMs (wild-type and IRF3 KO) in response to NA and ND chemical entity application. As a representative example, FIG. 22A depicts IRF3 dependent cytokine induction by NA-42 for cytokines IL6, MCP3, Rantes, and TNFa vs vehicle. FIG. 22B further shows IRF3 dependent cytokine induction by NA-42 for cytokines MCP1, Mip1b, Mip1a, and Gro-alpha vs vehicle. Also depicted for comparison at FIGS. 22A-22B is induction by IFNb and DMXAA. Along similar lines, FIG. 23A depicts IRF3 dependent cytokine induction by ND-13 for cytokines IL6, MCP3, Rantes, and TNFa vs vehicle. FIG. 23B further shows IRF3 dependent cytokine induction by ND-13 for cytokines MCP1, Mip1b, Mip1a, and Gro-alpha vs vehicle. Also depicted for comparison at FIGS. 23A-23B is induction by IFNb and DMXAA. The data depicted at FIGS. 22A-22B (related to NA-42) and the data depicted at FIGS. 23A-23B (related to ND-13) shows that both NA-42 and ND-13 treatment in mBMDMs are associated with minimal off-target inflammatory activity. Cytokine expression at each of FIGS. 22A-23B was monitored using a cytokine 20-plex mouse ProcartaPlex kit (Invitrogen, Carlsbad, Calif.) for labeling of multiple cytokines within an individual sample and run on a Luminex 200 instrument, and is expressed at FIGS. 22A-23B as pg/ml.

### Example 21

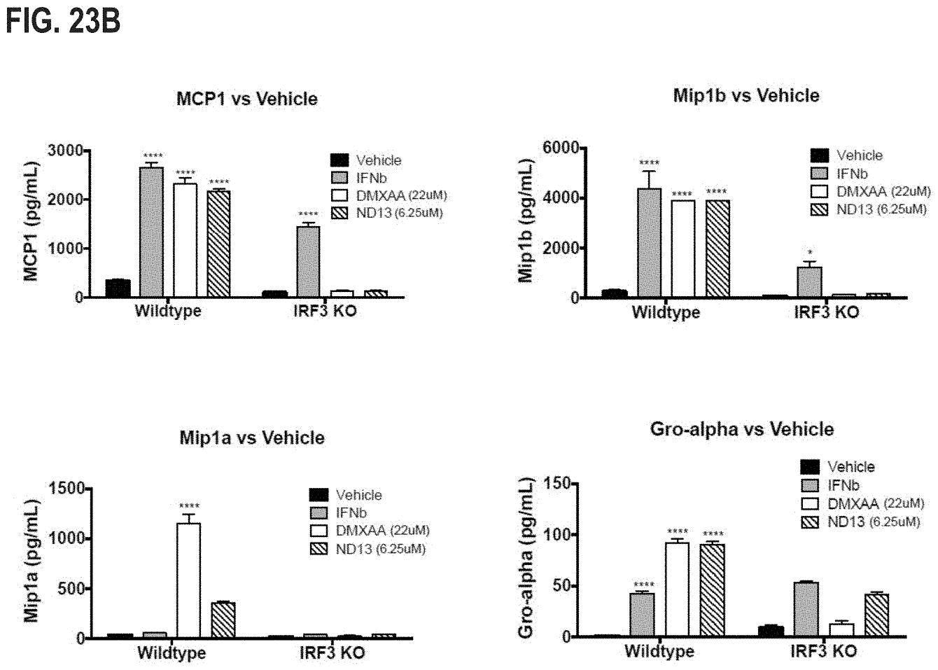

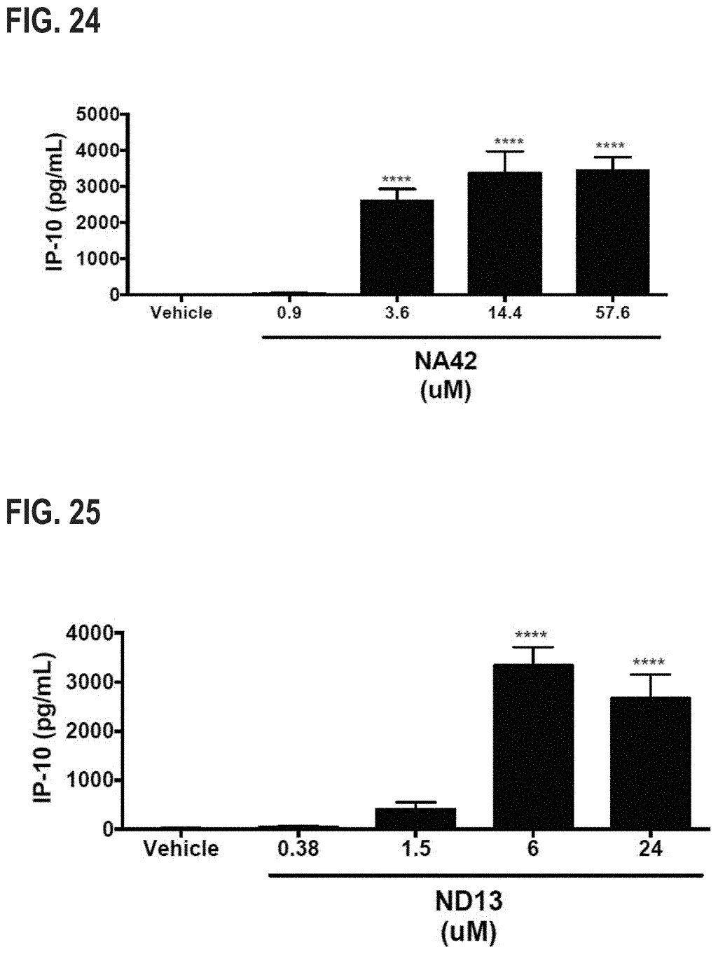

This example demonstrates target specificity in humans of compounds identified in the high-throughput screening methodology of the present disclosure. As discussed above with regard to Example 19, target specificity of chemical entities corresponding to the ND and NA chemofamilies was examined by dose-dependent expression of IP-10 in mBMDMs. In this example, 23, target specificity of chemical entities corresponding to the ND and NA chemofamiles was examined by determining dose-dependent expression of IP-10 in human primary cells, specifically monocyte-derived macrophages. As one representative example, FIG. 24 illustrates NA-42 dose-dependent IP-10 induction in human monocyte-derived macrophages. As another representative example, FIG. 25 illustrates ND-13 dose-dependent IP-10 induction in human monocyte-derived macrophages. For FIGS. 24-25, IP-10 expression was monitored by Human CXCL10/IP10 ELISA (R&D Systems, Minneapolis, Minn.), and is expressed at FIGS. 24-25 as pg/ml.

### Example 22

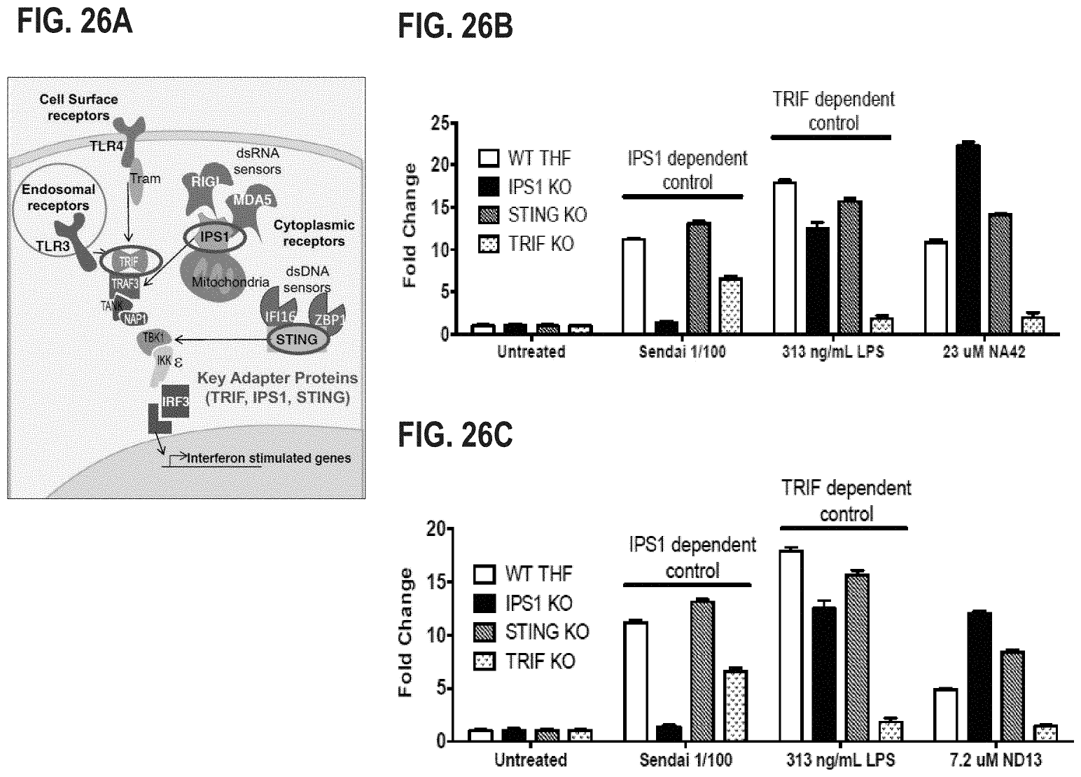

Select chemical entities corresponding to the NA and ND chemofamilies were examined as to their TRIF-dependency of IRF3 induction. TRIF, or TIR-domain-containing adapter-inducing interferon-β, is an adapter in responding to activation of toll-like receptors (TLRs). For reference, FIG. 26A illustrates a cell-signalling pathway including TRIF, along with TLR4, TLR3, IPS1, STING and IRF3. As a representative example, FIG. 26B shows fold-change of an interferon dependent luciferase reporter expressed in various telomerized human fibroblasts (THF) cell lines lacking key adapter molecules in the IRF3 activating pathway (WT THF, IPS1 KO, STING KO, and TRIF KO) in response to different treatments (untreated, Sendai 1/100, 313 ng/ml LPS, 23 μM NA-42). The treatment with Sendai 1/100 serves as an IPS1-dependent control, and the treatment with 313 ng/ml LPS serves as a TRIF-dependent control. As shown at FIG. 26B, treatment with 313 ng/ml LPS induces robust IRF3 induction in WT THF, IPS1 KO, and STING KO, but not in the TRIF KO, showing that LPS induction is TRIF-dependent. Treatment with NA-42 follows similar logic in that robust IRF3 induction is observed in response to 23 μM NA-42 in the WT THF, IPS1 KO, and STING KO, but not in the TRIF KO, showing that NA-42 activation of IRF3 is also TRIF dependent. Assessment of fold change IRF3 was determined via measurement of the interferon dependent luciferase reporter. FIG. 26C shows a similar data set obtained in similar fashion as that discussed with regard to FIG. 26B, with the exception that ND-13 was used as the test compound. Similar to NA-42 discussed above, ND-13 induction of IRF3 was shown to be TRIF-dependent.

### Example 23

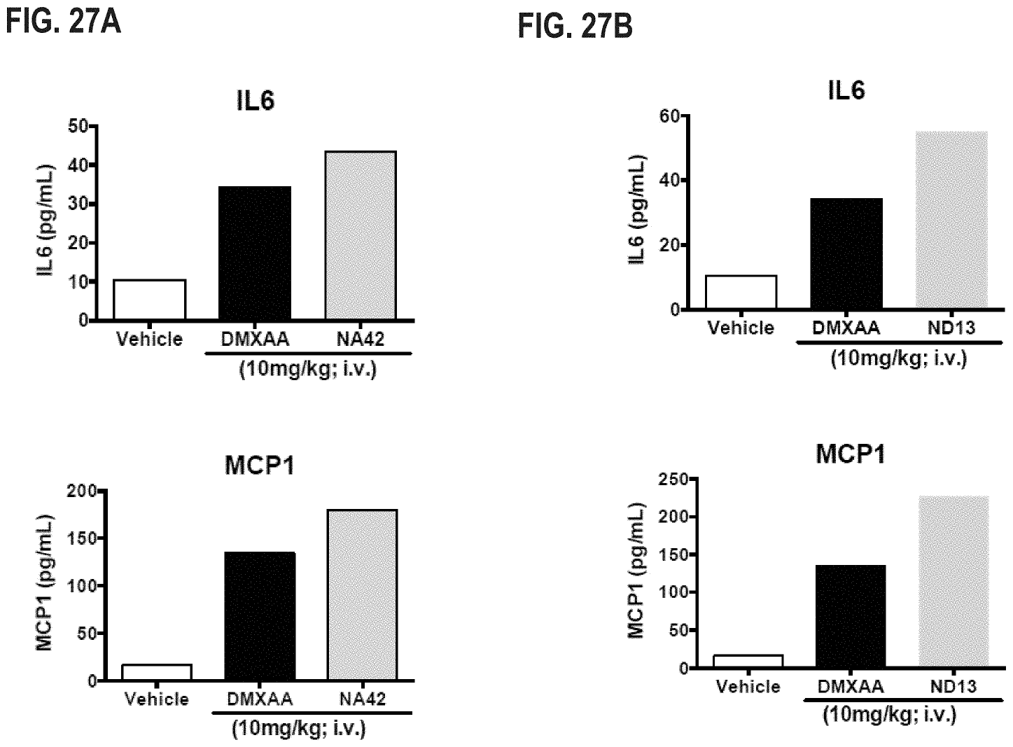

This example demonstrates IRF3 dependent cytokine induction in vivo by compounds identified via the high-throughput screening methodology of the present disclosure. Select chemical entities corresponding to the NA and ND chemofamilies were examined as to their ability to induce the expression of various cytokines in vivo. Specifically, WT C57/BL6 mice were given either vehicle, DMXAA, or select chemical entities corresponding to the NA and ND chemofamilies, and expression of relevant plasma cytokines was determined via a cytokine 20-plex mouse ProcartaPlex kit (Invitrogen) for labeling of multiple cytokines within an individual sample and run on a Luminex 200 instrument. Shown at FIG. 27A is a representative example showing NA-42 induction of IL-6 (top panel) and MCP1 (bottom panel). Similar to DMXAA, NA-42 is shown to induce robust expression of IL-6 and MCP1 in vivo. For both the top panel and the bottom panel at FIG. 27A, DMXAA and NA-42 were provided to the mice at 10 mg/kg, i.v.).

FIG. 27B shows a similar data set obtained in similar fashion as that discussed with regard to FIG. 27A, with the exception that ND-13 was used as the test compound. Similar to NA-42 discussed above, ND-13 was shown to induce robust IL-6 (top panel) and MCP1 (bottom panel) expression in vivo in mice.

### Example 24

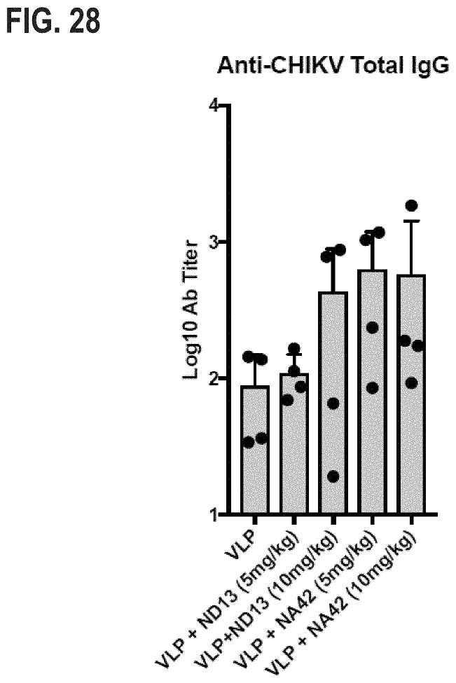

This example demonstrates the ability of compounds identified via the high throughput screening methodology of the present disclosure to enhance antibody titers to a Chikungunya Virus challenge. Select chemical entities corresponding to the NA and ND chemofamilies were examined as to their ability to act as adjuvants to enhance vaccine platforms. FIG. 28 depicts a representative example showing how NA-42 and ND-13 can enhance antibody titers to a Chikungunya Virus (CHIKV) challenge. C57BL/6 mice were primed and boosted IM with 10⁶ PFU equivalents of CHIKVLP alone or in the presence of ND13 or NA42; 3 weeks post-boost mice were euthanized and total IgG reactive with CHIKV was measured by ELISA in duplicate. Data presented are average geometric mean titers for each treatment cohort including each animal. Specifically, FIG. 28 depicts various conditions (virus-like particle (VLP), VLP+5 mg/kg ND13, VLP+10 mg/kg ND13, VLP+5 mg/kg NA-42, and VLP+10 mg/kg NA-42) plotted against Log 10 antibody titer representing Anti-CHIKV Total IgG. As shown, ND-13 at 10 mg/kg and NA-42 at both 5 mg/kg and 10 mg/kg robustly enhances antibody titers to CHIKV challenge, as compared to VLP alone.

### Example 25

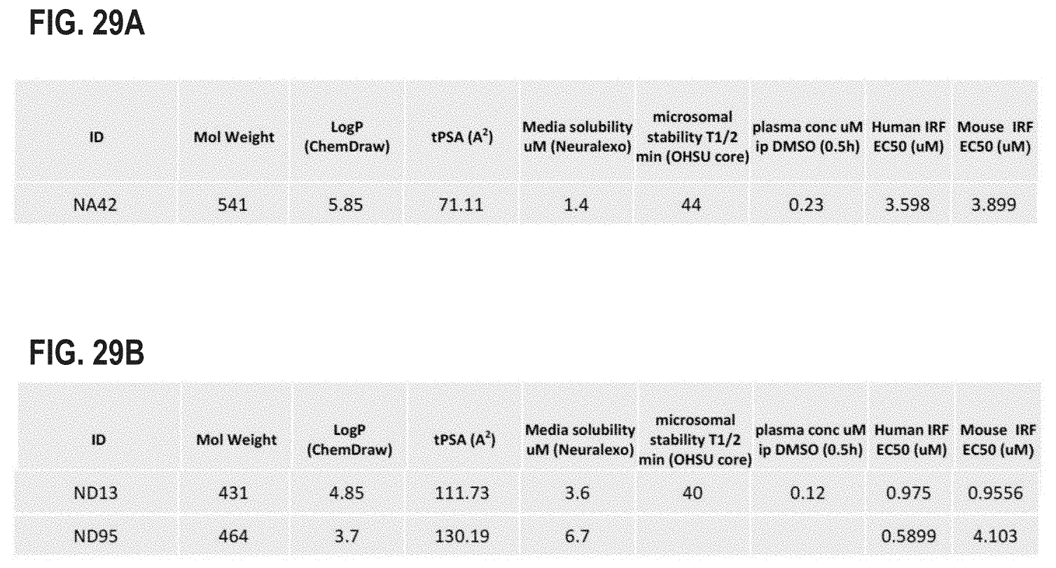

This example demonstrates various parameters related to compounds identified by the high throughput screening methodology of the present disclosure. Select chemical entities corresponding to the NA and ND chemofamilies were characterized according to, for example, molecular weight, Log P (partition coefficient) (ChemDraw), topological polar surface area (tPSA), media solubility (μM), microsomal stability (T % min), plasma concentration (μM IP DMSO, 0.5 h), human IRF EC50 (μM) and mouse IRF EC50 (μM). Shown as a representative example for the NA chemofamily at FIG. 29A is such characterization for NA-42. Shown as representative examples for the ND chemofamily at FIG. 29B is such characterization for ND-13, as well as ND-95.

Disclosed are compounds for altering activity and/or expression of an interferon regulatory factor (IRF) in a subject suffering from a condition or disease comprises one or more of formulas A-D:

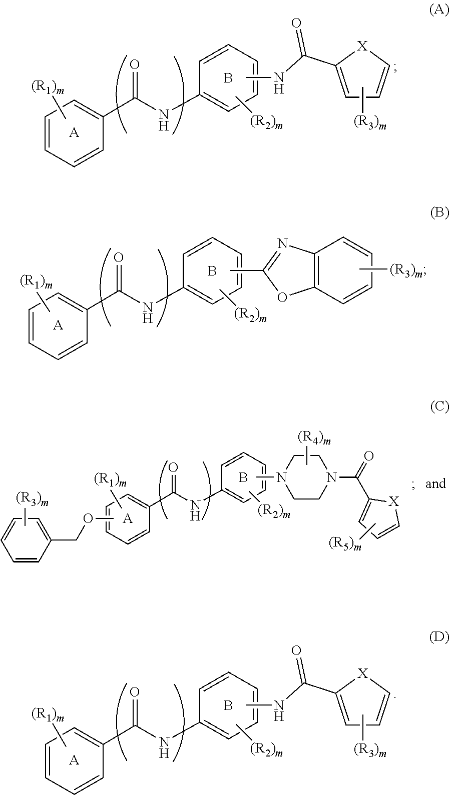

In such an embodiment, the compound or compounds may comprise molecule(s) selected from Table 7 and/or Table 8.

In such an embodiment, the IRF may be IRF3.

In such an embodiment, the IRF may be IRF7.

In such an embodiment, altering activity and/or expression of the IRF may comprise increasing the IRF activity and/or expression as compared to IRF activity and/or expression prior to administration of the IRF modulatory agent.

In such an embodiment, the condition or disease may be a stroke.

In such an embodiment, the condition or disease may be a cancer.

In such an embodiment, the condition or disease may be multiple sclerosis.

In such an embodiment, the condition or disease may be chronic fatigue syndrome.

In such an embodiment, the condition or disease may be an immune response to an antigen. In one example, the antigen is a chikungunya virus antigen.

In another embodiment, a compound or compounds for altering activity and/or expression of an interferon regulatory factor (IRF) in a subject suffering from a condition or disease comprises the following formula:

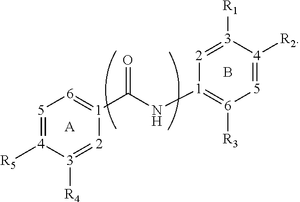

In such an embodiment, R1-R5 may comprise any combination of the following:

| R₁ | R₂ | R₃ | R₄ | R₅ |
|---|---|---|---|---|
| 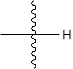 [SMILES: *[H]] | 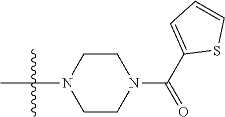 [SMILES: *N1CCN(C(=O)c2cccs2)CC1] | 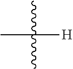 [SMILES: *[H]] | 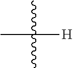 [SMILES: *[H]] | 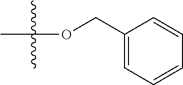 [SMILES: *OCc1ccccc1] |
| 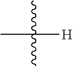 [SMILES: *[H]] | 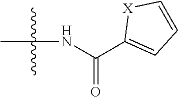 | 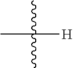 [SMILES: *[H]] | 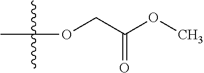 [SMILES: *OCC(=O)OC] | 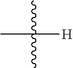 [SMILES: *[H]] |
| | | | 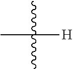 [SMILES: *[H]] | 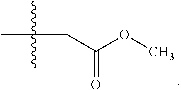 [SMILES: *CC(=O)OC] |

In such an embodiment, the compound or compounds may comprise molecule(s) selected from Table 7.

In such an embodiment, the IRF may be IRF3.

In such an embodiment, the IRF may be IRF7.

In such an embodiment, altering activity and/or expression of the IRF may comprise increasing the IRF activity and/or expression as compared to IRF activity and/or expression prior to administration of the IRF modulatory agent.

In such an embodiment, the condition or disease may be a stroke.

In such an embodiment, the condition or disease may be a cancer.

In such an embodiment, the condition or disease may be multiple sclerosis.

In such an embodiment, the condition or disease may be chronic fatigue syndrome.

In such an embodiment, the condition or disease may be an immune response to an antigen. In one example, the antigen is a chikungunya virus antigen.

In another embodiment, a compound or compounds for altering activity and/or expression of an interferon regulatory factor (IRF) in a subject suffering from a condition or disease comprises the following formula:

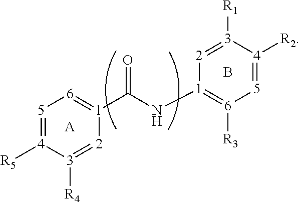

In such an embodiment, R1-R5 may comprise any combination of the following:

| R₁ | R₂ | R₃ | R₄ | R₅ |
|---|---|---|---|---|
| 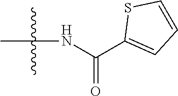 [SMILES: *NC(=O)c1cccs1] | 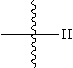 [SMILES: *[H]] | 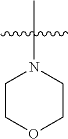 [SMILES: *N1CCOCC1] | halogen | 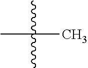 |
| 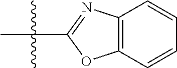 [SMILES: *c1nc2ccccc2o1] | 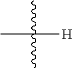 [SMILES: *[H]] | 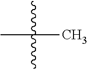 | 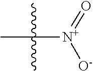 [SMILES: *[N+](=O)[O-]] | 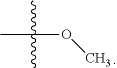 [SMILES: *OC] |

In such an embodiment, the compound or compounds may comprise molecule(s) selected from Table 8.

In such an embodiment, the IRF may be IRF3.

In such an embodiment, the IRF may be IRF7.

In such an embodiment, altering activity and/or expression of the IRF may comprise increasing the IRF activity and/or expression as compared to IRF activity and/or expression prior to administration of the IRF modulatory agent.

In such an embodiment, the condition or disease may be a stroke.

In such an embodiment, the condition or disease may be a cancer.

In such an embodiment, the condition or disease may be multiple sclerosis.

In such an embodiment, the condition or disease may be chronic fatigue syndrome.

In such an embodiment, the condition or disease may be an immune response to an antigen. In one example, the antigen is a chikungunya virus antigen.

In another embodiment, a compound or compounds for altering activity and/or expression of an interferon regulatory factor (IRF) in a subject suffering from a condition or disease comprises the following formula:

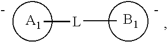

where A1 and B1 are functional groups and L is a linker region.

In such an embodiment, A1 may comprise one of:

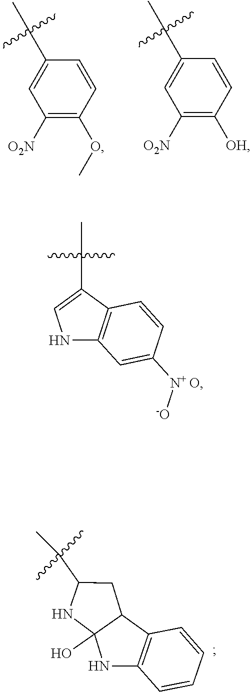

and

B1 may comprise.

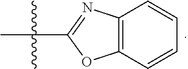 [SMILES: *c1nc2ccccc2o1]

In another embodiment, A1 may comprise one of:

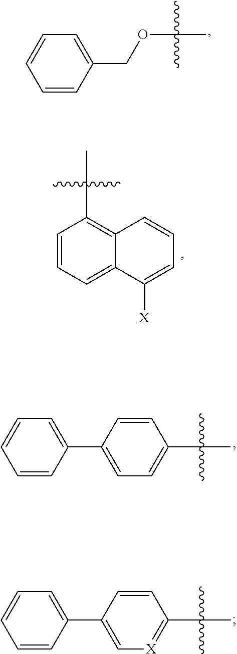

and B1 may comprise one of

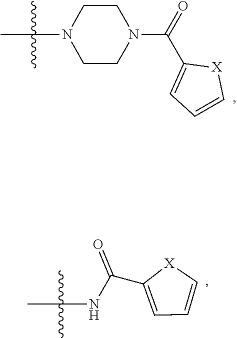

where X is one of carbon, nitrogen, oxygen, or sulfur.

In such an embodiment, the IRF may be IRF3.

In such an embodiment, the IRF may be IRF7.

In such an embodiment, altering activity and/or expression of the IRF may comprise increasing the IRF activity and/or expression as compared to IRF activity and/or expression prior to administration of the IRF modulatory agent.

In such an embodiment, the condition or disease may be a stroke.

In such an embodiment, the condition or disease may be a cancer.

In such an embodiment, the condition or disease may be multiple sclerosis.

In such an embodiment, the condition or disease may be chronic fatigue syndrome.

In such an embodiment, the condition or disease may be an immune response to an antigen. In one example, the antigen is a chikungunya virus antigen. In such an embodiment, L may comprise a reversible amide bond.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A method of altering activity and/or expression of an interferon regulatory factor (IRF) in a subject suffering from a condition or disease, comprising:
administering to the subject an effective amount of an IRF modulatory agent, the IRF modulatory agent comprising one or more compounds of Table 7 and/or Table 8.

2. The method of claim 1, wherein the IRF is IRF3.

3. The method of claim 1, wherein the IRF is IRF7.

4. The method of claim 1, wherein altering activity and/or expression of the IRF comprises increasing the IRF activity and/or expression as compared to IRF activity and/or expression prior to administration of the IRF modulatory agent.

5. The method of claim 1, wherein the condition or disease is a stroke.

6. The method of claim 5, wherein administration of the IRF modulatory agent improves a tolerance of neural tissue in the subject to an ischemic event associated with the stroke as compared to the tolerance in the absence of administration of the IRF modulatory agent.

7. The method of claim 5, further comprising providing the subject with a thrombolytic therapy within a first predetermined time period of administration of the effective amount of the IRF modulatory agent.

8. The method of claim 5, further comprising performing a surgical thrombectomy on the subject to remove a blood clot from inside an artery or a vein of the subject within a second predetermined time period of administration of the effective amount of the IRF modulatory agent.

9. The method of claim 1, wherein the condition or disease is an immune response to an antigen.

10. The method of claim 9, wherein the IRF modulatory agent acts as an adjuvant to potentiate and/or modulate the immune response to the antigen.

11. The method of claim 9, wherein the antigen is a chikungunya virus antigen.

12. A method of treating subject having a condition/disorder or disease that is at least in part regulated by activity and/or expression of an interferon regulatory factor (IRF), comprising:
contacting a cell or cells of the subject with an effective amount of an IRF modulatory agent, the IRF modulatory agent comprising one or more compounds of Table 7 and/or Table 8, to increase the activity and/or expression of the IRF, thereby treating the condition/disorder or disease.

13. The method of claim 12, wherein the IRF is IRF3.

14. The method of claim 12, wherein the IRF is IRF7.

15. The method of claim 12, wherein increasing the activity and/or expression of the IRF is in relation to IRF activity and/or expression prior to or in the absence of the cell or cells being contacted with the IRF modulatory agent.

16. The method of claim 12, wherein the condition/disorder or disease is a stroke.

17. The method of claim 16, wherein contacting the cell or cells of the subject with the IRF modulatory agent delays a depletion of cellular energy stores and delays membrane potential depolarization of the cell or cells affected by the stroke as compared to a rate at which depletion of cellular energy stores and membrane potential depolarization occurs in the absence of the cell or cells being contacted with the IRF modulatory agent.

18. The method of claim 16, further comprising administering to the subject a thrombolytic therapy within a first predetermined time period of the cell or cells being contacted with the IRF modulatory agent.

19. The method of claim 16, further comprising performing a surgical thrombectomy on the subject to remove a blood clot from inside an artery or a vein of the subject within a second predetermined time period of the cell or cells being contacted with the IRF modulatory agent.

20. A method of increasing a time frame of a therapeutic window in which one or more treatments can be effectively provided to a subject suffering from an acute ischemic event, comprising:
altering activity and/or expression of an interferon regulatory factor (IRF) by administering to the subject an IRF modulatory agent within a predetermined period of time of an initiation of the acute ischemic event.
